(19) United States
(12) Patent Application Publication
(10) Pub. No.: US 2020/0209741 A1
(43) Pub. Date: Jul. 2, 2020

(54) RESIST COMPOSITION, METHOD OF FORMING RESIST PATTERN, POLYMERIC COMPOUND, AND COMPOUND

(71) Applicant: TOKYO OHKA KOGYO CO., LTD., kawasaki-shi (JP)

(72) Inventors: Junichi TSUCHIYA, kawasaki-shi (JP); Kotaro ENDO, kawasaki-shi (JP); Masafumi FUJISAKI, kawasaki-shi (JP); Hideto NITO, kawasaki-shi (JP)

(21) Appl. No.: 16/642,825

(22) PCT Filed: Sep. 21, 2018
(86) PCT No.: PCT/JP2018/035065

§ 371 Date: Feb. 27, 2020

(30) Foreign Application Priority Data
Sep. 26, 2017 (JP) 2017-185010

(51) Int. Cl. G03F 7/039; G03F 7/038; G03F 7/16; G03F 7/20; G03F 7/38; G03F 7/32; C08F 220/18; C07D 327/04; C08F 220/38

(52) U.S. Cl. CPC G03F 7/039; G03F 7/038; G03F 7/168; G03F 7/2006; C08F 220/1818; G03F 7/325; C08F 220/1811; C07D 327/04; C08F 220/382; G03F 7/38

(57) ABSTRACT

A resist composition including a resin component (A1) which exhibits changed solubility in a developing solution under action of acid, the resin component (A1) including a structural unit (a0) represented by general formula (a0-1) shown below (wherein Ya⁰¹ and Ya⁰² each independently represents a single bond or an alkylene group of 1 to 3 carbon atoms, provided that the total number of carbon atoms of Ya⁰¹ and Ya⁰² is 3 or less; Ra⁰¹ and Ra⁰² each independently represents a hydrogen atom or an alkyl group of 1 to 4 carbon atoms, provided that the total number of carbon atoms of Ra⁰¹ and Ra⁰² is 2 to 6; A′ represents an alkylene group of 1 to 5 carbon atoms).

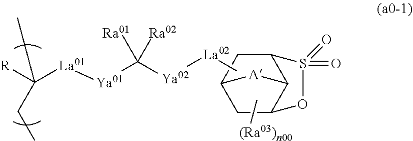

### TECHNICAL FIELD

The present invention relates to a resist composition, a method of forming a resist pattern, a polymeric compound, and a compound.

Priority is claimed on Japanese Patent Application No. 2017-185010, filed Sep. 26, 2017, the content of which is incorporated herein by reference.

### DESCRIPTION OF RELATED ART

In lithography techniques, for example, a resist film composed of a resist material is formed on a substrate, and the resist film is subjected to selective exposure, followed by development, thereby forming a resist pattern having a predetermined shape on the resist film. A resist material in which the exposed portions of the resist film become soluble in a developing solution is called a positive-type, and a resist material in which the exposed portions of the resist film become insoluble in a developing solution is called a negative-type.

In recent years, in the production of semiconductor elements and liquid crystal display elements, advances in lithography techniques have led to rapid progress in the field of pattern miniaturization. Typically, these miniaturization techniques involve shortening the wavelength (increasing the energy) of the exposure light source. Conventionally, ultraviolet radiation typified by g-line and i-line radiation has been used, but nowadays KrF excimer lasers and ArF excimer lasers are used in mass production. Furthermore, research is also being conducted into lithography techniques that use an exposure light source having a wavelength shorter (energy higher) than these excimer lasers, such as electron beam (EB), extreme ultraviolet radiation (EUV), and X ray.

Resist materials for use with these types of exposure light sources require lithography properties such as a high resolution capable of reproducing patterns of minute dimensions, and a high level of sensitivity to these types of exposure light sources.

As a resist material that satisfies these conditions, a chemically amplified composition is used, which includes a base material component that exhibits a changed solubility in a developing solution under the action of acid and an acid-generator component that generates acid upon exposure.

For example, in the case where the developing solution is an alkali developing solution (alkali developing process), a chemically amplified positive resist which contains, as a base component (base resin), a resin which exhibits increased solubility in an alkali developing solution under action of acid, and an acid generator is typically used. If a resist film formed using such a resist composition is selectively exposed at the time of forming a resist pattern, in exposed areas, acid is generated from the acid generator component, and the polarity of the base resin increases by the action of the generated acid, thereby making the exposed areas of the resist film soluble in the alkali developing solution. Thus, by alkali developing, the exposed portions of the resist film are dissolved and removed, whereas the unexposed portions of the resist film remain as patterns, thereby forming a positive resist pattern.

On the other hand, when such a base resin is applied to a solvent developing process using a developing solution containing an organic solvent (organic developing solution), the solubility of the exposed portions in an organic developing solution is decreased. As a result, the unexposed portions of the resist film are dissolved and removed by the organic developing solution, and a negative resist pattern in which the exposed portions of the resist film are remaining is formed. Such a solvent developing process for forming a negative-tone resist composition is sometimes referred to as “negative-tone developing process”.

In general, the base resin used for a chemically amplified resist composition contains a plurality of structural units for improving lithography properties and the like.

For example, in the case of a resin composition which exhibits increased solubility in an alkali developing solution by the action of acid, a structural unit containing an acid decomposable group which is decomposed by the action of acid generated from an acid generator component and exhibits increased polarity. Further, a structural unit containing a lactone-containing cyclic group or a structural unit containing a polar group such as a hydroxy group is used in combination.

Further, in order to improve various lithography properties, a resin having a structural unit containing a —SO₂-containing cyclic group has been used (for example, see Patent Literatures 1 to 3).

### DOCUMENTS OF RELATED ART

### Patent Literature

[Patent Document 1] Japanese Unexamined Patent Application, First Publication No. 2009-062491

[Patent Document 2] Japanese Unexamined Patent Application, First Publication No. 2010-134417

[Patent Document 3] Japanese Unexamined Patent Application, First Publication No. 2013-225125

### SUMMARY OF THE INVENTION

### Problems to be Solved by the Invention

Since the structural unit containing a —SO₂-containing cyclic group has high resistance to a rinsing solution of an alcohol solvent such as methyl isobutyl carbinol (MIBC), it is desired to increase the amount of such structural unit in the base resin. However, there is a problem that the structural unit containing a —SO₂-containing cyclic group has low solubility in a developer containing an organic solvent. Therefore, in the case where a chemically amplified resist composition containing a resin having a structural unit containing a —SO₂-containing cyclic group as a base resin is applied to a solvent development process, when the amount of the structural unit containing a —SO₂-containing cyclic group is increased, there is a possibility that lithography properties may be adversely affected.

The present invention takes the above circumstances into consideration, with an object of providing a resist composition exhibiting good lithography properties, a method of forming a resist pattern using the resist composition, a polymer compound useful as a resin component for the resist composition, and a compound useful for producing the polymeric compound.

### Means to Solve the Problems

For solving the above-mentioned problems, the present invention employs the following aspects.

Specifically, a first aspect of the present invention is a resist composition which generates acid upon exposure and exhibits changed solubility in a developing solution under action of acid, the resist composition including: a resin component (A1) which exhibits changed solubility in a developing solution under action of acid, the resin component (A1) including a structural unit (a0) represented by general formula (a0-1) shown below.

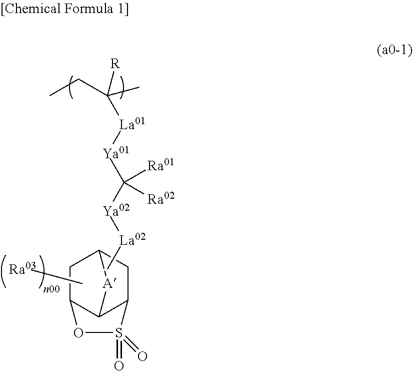

In the formula, R represents a hydrogen atom, an alkyl group of 1 to 5 carbon atoms or a halogenated alkyl group of 1 to 5 carbon atoms; La⁰¹ and La⁰² each independently represents a single bond or a divalent linking group;

Ya⁰¹ and Ya⁰² each independently represents a single bond or an alkylene group of 1 to 3 carbon atoms;

provided that the total number of carbon atoms of Ya⁰¹ and Ya⁰² is 3 or less; Ra⁰¹ and Ra⁰² each independently represents a hydrogen atom or an alkyl group of 1 to 4 carbon atoms; Ra⁰¹ and Ra⁰² may be mutually bonded to form a ring; provided that the total number of carbon atoms of Ra⁰¹ and Ra⁰² is 2 to 6; Ra⁰³ represents a hydrogen atom, an alkyl group, an alkoxy group, a halogen atom, a halogenated alkyl group, a hydroxy group, —COOR″, —OC(═O)R″, a hydroxyalkyl group or a cyano group; R″ represents a hydrogen atom, an alkyl group, a lactone-containing cyclic group, a carbonate-containing cyclic group or a —SO₂-containing cyclic group; n₀₀ represents an integer of 0 to 2;

when n₀₀ is 2, the plurality of Ra⁰³ may be the same or different from each other; and A′ represents an alkylene group of 1 to 5 carbon atoms.

A second aspect of the present invention is a method of forming a resist pattern, including: using a resist composition according to the first aspect to form a resist film, exposing the resist film, and developing the exposed resist film using a developing solution containing an organic solvent to form a resist pattern.

A third aspect of the present invention is a polymeric compound having a structural unit (a0) represented by general formula (a0-1) shown below.

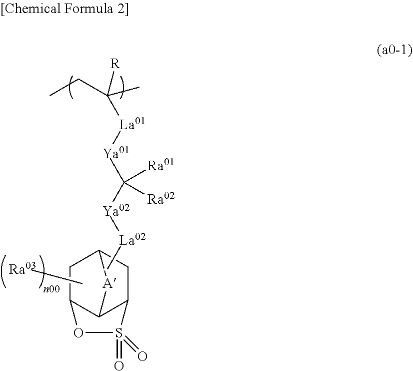

In the formula, R represents a hydrogen atom, an alkyl group of 1 to 5 carbon atoms or a halogenated alkyl group of 1 to 5 carbon atoms; La⁰¹ and La⁰² each independently represents a single bond or a divalent linking group;

Ya⁰¹ and Ya⁰² each independently represents a single bond or an alkylene group of 1 to 3 carbon atoms;

provided that the total number of carbon atoms of Ya⁰¹ and Ya⁰² is 3 or less; Ra⁰¹ and Ra⁰² each independently represents a hydrogen atom or an alkyl group of 1 to 4 carbon atoms; Ra⁰¹ and Ra⁰² may be mutually bonded to form a ring; provided that the total number of carbon atoms of Ra⁰¹ and Ra⁰² is 2 to 6; Ra⁰³ represents a hydrogen atom, an alkyl group, an alkoxy group, a halogen atom, a halogenated alkyl group, a hydroxy group, —COOR″, —OC(═O)R″, a hydroxyalkyl group or a cyano group; R″ represents a hydrogen atom, an alkyl group, a lactone-containing cyclic group, a carbonate-containing cyclic group or a —SO₂-containing cyclic group; n₀₀ represents an integer of 0 to 2;

when n₀₀ is 2, the plurality of Ra⁰³ may be the same or different from each other;

and A′ represents an alkylene group of 1 to 5 carbon atoms.

A fourth aspect of the present invention is a compound represented by general formula (m-a0) shown below.

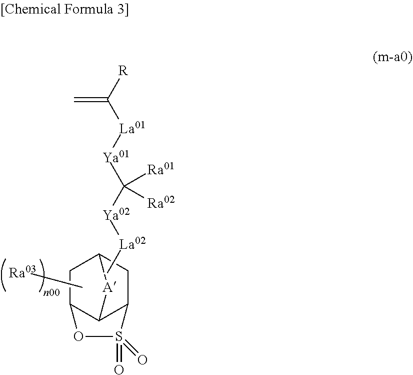

In the formula, R represents a hydrogen atom, an alkyl group of 1 to 5 carbon atoms or a halogenated alkyl group of 1 to 5 carbon atoms; La⁰¹ and La⁰² each independently represents a single bond or a divalent linking group;

Ya⁰¹ and Ya⁰² each independently represents a single bond or an alkylene group of 1 to 3 carbon atoms;

provided that the total number of carbon atoms of Ya⁰¹ and Ya⁰² is 3 or less; Ra⁰¹ and Ra⁰² each independently represents a hydrogen atom or an alkyl group of 1 to 4 carbon atoms; Ra⁰¹ and Ra⁰² may be mutually bonded to form a ring; provided that the total number of carbon atoms of Ra⁰¹ and Ra⁰² is 2 to 6; Ra⁰³ represents a hydrogen atom, an alkyl group, an alkoxy group, a halogen atom, a halogenated alkyl group, a hydroxy group, —COOR″, —OC(═O)R″, a hydroxyalkyl group or a cyano group; R″ represents a hydrogen atom, an alkyl group, a lactone-containing cyclic group, a carbonate-containing cyclic group or a —SO₂-containing cyclic group; n₀₀ represents an integer of 0 to 2;

when n₀₀ is 2, the plurality of Ra⁰³ may be the same or different from each other; and A′ represents an alkylene group of 1 to 5 carbon atoms.

### Effect of the Invention

According to the present invention, there are provided a resist composition exhibiting good lithography properties, a method of forming a resist pattern using the resist composition, a polymer compound useful as a resin component for the resist composition, and a compound useful for producing the polymeric compound.

### DETAILED DESCRIPTION OF THE INVENTION

In the present description and claims, the term “aliphatic” is a relative concept used in relation to the term “aromatic”, and defines a group or compound that has no aromaticity.

The term “alkyl group” includes linear, branched or cyclic, monovalent saturated hydrocarbon, unless otherwise specified. The same applies for the alkyl group within an alkoxy group.

The term “alkylene group” includes linear, branched or cyclic, divalent saturated hydrocarbon, unless otherwise specified.

A “halogenated alkyl group” is a group in which part or all of the hydrogen atoms of an alkyl group is substituted with a halogen atom. Examples of the halogen atom include a fluorine atom, a chlorine atom, a bromine atom and an iodine atom.

A “fluorinated alkyl group” or a “fluorinated alkylene group” is a group in which part or all of the hydrogen atoms of an alkyl group or an alkylene group have been substituted with a fluorine atom.

The term “structural unit” refers to a monomer unit that contributes to the formation of a polymeric compound (resin, polymer, copolymer).

The expression “may have a substituent” means that a case where a hydrogen atom (—H) is substituted with a monovalent group, or a case where a methylene (—CH₂—) group is substituted with a divalent group.

The term “exposure” is used as a general concept that includes irradiation with any form of radiation.

A “structural unit derived from an acrylate ester” refers to a structural unit that is formed by the cleavage of the ethylenic double bond of an acrylate ester.

An “acrylate ester” refers to a compound in which the terminal hydrogen atom of the carboxy group of acrylic acid (CH₂═CH—COOH) has been substituted with an organic group.

The acrylate ester may have the hydrogen atom bonded to the carbon atom on the α-position substituted with a substituent. The substituent (Ra^{α0}) that substitutes the hydrogen atom bonded to the carbon atom on the α-position is an atom other than hydrogen or a group, and examples thereof include an alkyl group of 1 to 5 carbon atoms and a halogenated alkyl group of 1 to 5 carbon atoms. Further, an acrylate ester having the hydrogen atom bonded to the carbon atom on the α-position substituted with a substituent (Ra^{α0}) in which the substituent has been substituted with a substituent containing an ester bond (e.g., an itaconic acid diester), or an acrylic acid having the hydrogen atom bonded to the carbon atom on the α-position substituted with a substituent) (Ra^{α0}) in which the substituent has been substituted with a hydroxyalkyl group or a group in which the hydroxy group within a hydroxyalkyl group has been modified (e.g., α-hydroxyalkyl acrylate ester) may be mentioned as an acrylate ester having the hydrogen atom bonded to the carbon atom on the α-position substituted with a substituent. A carbon atom on the α-position of an acrylate ester refers to the carbon atom bonded to the carbonyl group, unless specified otherwise.

Hereafter, an acrylate ester having the hydrogen atom bonded to the carbon atom on the α-position substituted with a substituent is sometimes referred to as “α-substituted acrylate ester”. Further, acrylate esters and α-substituted acrylate esters are collectively referred to as “(α-substituted) acrylate ester”. Hereafter, an acrylate ester having the hydrogen atom bonded to the carbon atom on the α-position substituted with a substituent is sometimes referred to as “α-substituted acrylate ester”. Further, acrylic acid and α-substituted acrylic acids are collectively referred to as “(α-substituted) acrylic acid”.

A “structural unit derived from acrylamide” refers to a structural unit that is formed by the cleavage of the ethylenic double bond of acrylamide.

The acrylamide may have the hydrogen atom bonded to the carbon atom on the α-position substituted with a substituent, and may have either or both terminal hydrogen atoms on the amino group of acrylamide substituted with a substituent. A carbon atom on the α-position of an acrylamide refers to the carbon atom bonded to the carbonyl group, unless specified otherwise.

As the substituent which substitutes the hydrogen atom on the α-position of acrylamide, the same substituents as those described above for the substituent (Ra^{α0}) on the α-position of the aforementioned α-position of the aforementioned α-substituted acrylate ester may be mentioned.

A “structural unit derived from hydroxystyrene” refers to a structural unit that is formed by the cleavage of the ethylenic double bond of hydroxystyrene. A “structural unit derived from a hydroxystyrene derivative” refers to a structural unit that is formed by the cleavage of the ethylenic double bond of a hydroxystyrene derivative.

The term “hydroxystyrene derivative” includes compounds in which the hydrogen atom at the α-position of hydroxystyrene has been substituted with another substituent such as an alkyl group or a halogenated alkyl group; and derivatives thereof. Examples of the derivatives thereof include hydroxystyrene in which the hydrogen atom of the hydroxy group has been substituted with an organic group and may have the hydrogen atom on the α-position substituted with a substituent; and hydroxystyrene which has a substituent other than a hydroxy group bonded to the benzene ring and may have the hydrogen atom on the α-position substituted with a substituent. Here, the α-position (carbon atom on the α-position) refers to the carbon atom having the benzene ring bonded thereto, unless specified otherwise.

As the substituent which substitutes the hydrogen atom on the α-position of hydroxystyrene, the same substituents as those described above for the substituent on the α-position of the aforementioned α-substituted acrylate ester may be mentioned.

A “structural unit derived from vinylbenzoic acid or a vinylbenzoic acid derivative” refers to a structural unit that is formed by the cleavage of the ethylenic double bond of vinylbenzoic acid or a vinylbenzoic acid derivative.

The term “vinylbenzoic acid derivative” includes compounds in which the hydrogen atom at the α-position of vinylbenzoic acid has been substituted with another substituent such as an alkyl group or a halogenated alkyl group; and derivatives thereof. Examples of the derivatives thereof include benzoic acid in which the hydrogen atom of the carboxy group has been substituted with an organic group and may have the hydrogen atom on the α-position substituted with a substituent; and benzoic acid which has a substituent other than a hydroxy group and a carboxy group bonded to the benzene ring and may have the hydrogen atom on the α-position substituted with a substituent. Here, the α-position (carbon atom on the α-position) refers to the carbon atom having the benzene ring bonded thereto, unless specified otherwise.

As the alkyl group as a substituent on the α-position, a linear or branched alkyl group is preferable, and specific examples include alkyl groups of 1 to 5 carbon atoms, such as a methyl group, an ethyl group, a propyl group, an isopropyl group, an n-butyl group, an isobutyl group, a tert-butyl group, a pentyl group, an isopentyl group and a neopentyl group.

Specific examples of the halogenated alkyl group as the substituent on the α-position include groups in which part or all of the hydrogen atoms of the aforementioned “alkyl group as the substituent on the α-position” are substituted with halogen atoms. Examples of the halogen atom include a fluorine atom, a chlorine atom, a bromine atom and an iodine atom, and a fluorine atom is particularly desirable.

Specific examples of the hydroxyalkyl group as the substituent on the α-position include groups in which part or all of the hydrogen atoms of the aforementioned “alkyl group as the substituent on the α-position” are substituted with a hydroxy group. The number of hydroxy groups within the hydroxyalkyl group is preferably 1 to 5, and most preferably 1.

In the present specification and claims, some structures represented by chemical formulae may have an asymmetric carbon, such that an enantiomer or a diastereomer may be present. In such a case, the one formula represents all isomers. The isomers may be used individually, or in the form of a mixture.

(Resist composition)

The resist composition of the present embodiment generates acid upon exposure and exhibits changed solubility in a developing solution under action of acid.

The resist composition contains a base component (A) (hereafter, sometimes referred to as “base component (A)”) which exhibits changed solubility in a developing solution.

When a resist film is formed using the resist composition according to the present embodiment, and the resist film is selectively exposed, acid is generated at exposed portions of the resist film, and the solubility of the component (A) in a developing solution is changed by the action of the acid. On the other hand, at unexposed portions of the resist film, the solubility of the component (A) in a developing solution is unchanged. As a result, difference is generated between the exposed portions of the resist film and the unexposed portions of the resist film in terms of solubility in a developing solution. Therefore, by subjecting the resist film to development, the exposed portions of the resist film are dissolved and removed to form a positive-tone resist pattern in the case of a positive resist, whereas the unexposed portions of the resist film are dissolved and removed to form a negative-tone resist pattern in the case of a negative resist.

In the present specification, a resist composition which forms a positive resist pattern by dissolving and removing the exposed portions of the resist film is called a positive resist composition, and a resist composition which forms a negative resist pattern by dissolving and removing the unexposed portions of the resist film is called a negative resist composition.

The resist composition of the present embodiment may be either a positive resist composition or a negative resist composition.

Further, in the present embodiment, the resist composition may be applied to an alkali developing process using an alkali developing solution in the developing treatment, or a solvent developing process using a developing solution containing an organic solvent (organic developing solution) in the developing treatment, and preferably a solvent developing process.

That is, the resist composition of the present embodiment is preferably a resist composition which forms a positive pattern in an alkali developing process (i.e, a positive resist compound for alkali developing process) or a resist composition which forms a negative pattern in a solvent developing process (i.e., a negative type resist composition for solvent developing process).

The resist composition of the present embodiment is capable of generating acid upon exposure. The acid may be generated from the component (A) upon exposure, or the acid may be generated from an additive component other than the component (A) upon exposure.

More specifically, the resist composition according to the present embodiment may be

a resist composition (1) containing an acid generator component (B) which generates acid upon exposure (hereafter, referred to as “component (B)”;

a resist composition (2) in which the component (A) is a component which generates acid upon exposure; or

a resist composition (3) in which the component (A) is a component which generates acid upon exposure, and further containing an acid generator component (B).

That is, when the resist composition of the present invention is the aforementioned resist composition (2) or (3), the component (A) is a “base component which generates acid upon exposure and exhibits changed solubility in a developing solution under action of acid”. In the case where the component (A) is a base component which generates acid upon exposure and exhibits changed solubility in a developing solution under action of acid, the component (A1) described later is preferably a polymeric compound which generates acid upon exposure and exhibits changed solubility in a developing solution under action of acid. As the polymeric compound, a resin having a structural unit which generates acid upon exposure may be used.

As the structural unit which generates acid upon exposure, a conventional structural unit may be used.

The resist composition of the present embodiment is most preferably the aforementioned resist composition (1).

<Component (A)>

The component (A) is a base component which exhibits changed solubility in a developing solution under action of acid.

In the present invention, the term “base component” refers to an organic compound capable of forming a film, and is preferably an organic compound having a molecular weight of 500 or more. When the organic compound has a molecular weight of 500 or more, the film-forming ability is improved, and a resist pattern of nano level may be easily formed.

The organic compound used as the base component is broadly classified into non-polymers and polymers.

In general, as a non-polymer, any of those which have a molecular weight in the range of 500 to less than 4,000 is used. Hereafter, a “low molecular weight compound” refers to a non-polymer having a molecular weight in the range of 500 to less than 4,000.

As a polymer, any of those which have a molecular weight of 1,000 or more is generally used. Hereafter, a “resin” or a “polymer” refers to a polymer having a molecular weight of 1,000 or more.

As the molecular weight of the polymer, the weight average molecular weight in terms of the polystyrene equivalent value determined by gel permeation chromatography (GPC) is used.

In the resist composition according to the present embodiment, the component (A) contains a resin component (A1) (hereafter, referred to as “component (A1)”) which exhibits changed solubility in a developing solution by the action of acid.

As the component (A), at least the component (A1) is used, and a polymeric compound and/or a low molecular weight compound may be used in combination with the component (A1).

<<Component (A1)>>

The component (A1) includes a structural unit (a0) represented by general formula (a0-1) described later. If desired, the component (A1) may include, in addition to the structural unit (a0), other structural unit.

Examples of the component (A1) include a copolymer having a structural unit (a0), and any other structural unit if desired (hereafter, this copolymer is sometimes referred to as “component (A1-1)”).

In the resist composition of the present embodiment, by using the component (A1), the polarity of the resin component changes before and after exposure. Therefore, an excellent development contrast may be obtained between exposed portions and unexposed portions of the resist film not only in an alkali developing process, but also in a solvent developing process. The resist composition of the present embodiment is particularly preferably applied to a solvent development process because the solubility of the structural unit (a0) in an organic developer is good.

More specifically, in the case of applying an alkali developing process, the component (A1) is substantially insoluble in an alkali developing solution prior to exposure, but when acid is generated from the component (B) upon exposure, the action of this acid causes an increase in the polarity of the base component, thereby increasing the solubility of the component (A1) in an alkali developing solution. Therefore, in the formation of a resist pattern, by conducting selective exposure of a resist film formed by applying the resist composition to a substrate, the exposed portions of the resist film change from an insoluble state to a soluble state in an alkali developing solution, whereas the unexposed portions of the resist film remain insoluble in an alkali developing solution, and hence, a positive resist pattern is formed by alkali developing.

On the other hand, in the case of a solvent developing process, the component (A1) exhibits high solubility in an organic developing solution prior to exposure, and when acid is generated from the component (B) upon exposure for example, the polarity of the component (A1) is increased by the action of the generated acid, thereby decreasing the solubility of the component (A1) in an organic developing solution. Therefore, in the formation of a resist pattern, by conducting selective exposure of a resist film formed by applying the resist composition to a substrate, the exposed portions of the resist film changes from an soluble state to an insoluble state in an organic developing solution, whereas the unexposed portions of the resist film remain soluble in an organic developing solution. As a result, by conducting development using an organic developing solution, thereby forming a negative resist pattern.

[Structural Unit (a0)]

The structural unit (a0) is a structural unit represented by general formula (a0-1) shown below.

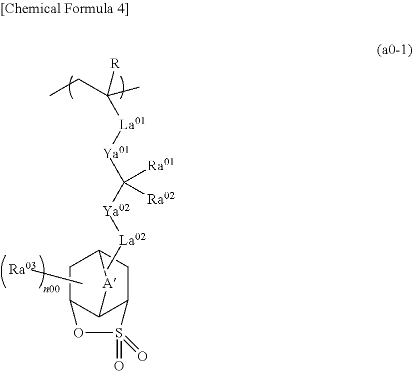

In the formula, R represents a hydrogen atom, an alkyl group of 1 to 5 carbon atoms or a halogenated alkyl group of 1 to 5 carbon atoms; La⁰¹ and La⁰² each independently represents a single bond or a divalent linking group;

Ya⁰¹ and Ya⁰² each independently represents a single bond or an alkylene group of 1 to 3 carbon atoms;

provided that the total number of carbon atoms of Ya⁰¹ and Ya⁰² is 3 or less; Ra⁰¹ and Ra⁰² each independently represents a hydrogen atom or an alkyl group of 1 to 4 carbon atoms; Ra⁰¹ and Ra⁰² may be mutually bonded to form a ring; provided that the total number of carbon atoms of Ra⁰¹ and Ra⁰² is 2 to 6; Ra⁰³ represents a hydrogen atom, an alkyl group, an alkoxy group, a halogen atom, a halogenated alkyl group, a hydroxy group, —COOR″, —OC(═O)R″, a hydroxyalkyl group or a cyano group; R″ represents a hydrogen atom, an alkyl group, a lactone-containing cyclic group, a carbonate-containing cyclic group or a —SO₂-containing cyclic group; n₀₀ represents an integer of 0 to 2;

when n₀₀ is 2, the plurality of Ra⁰³ may be the same or different from each other; and A′ represents an alkylene group of 1 to 5 carbon atoms.

In general formula (a0-1), R represents a hydrogen atom, an alkyl group of 1 to 5 carbon atoms or a halogenated alkyl group of 1 to 5 carbon atoms.

As the alkyl group having 1 to 5 carbon atoms for R, a linear or branched alkyl group of 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group and a neopentyl group.

The halogenated alkyl group of 1 to 5 carbon atoms represented by R is a group in which part or all of the hydrogen atoms of the aforementioned alkyl group of 1 to 5 carbon atoms have been substituted with halogen atoms. Examples of the halogen atom include a fluorine atom, a chlorine atom, a bromine atom and an iodine atom, and a fluorine atom is particularly desirable.

As R, a hydrogen atom, an alkyl group of 1 to 5 carbon atoms or a fluorinated alkyl group of 1 to 5 carbon atoms is preferable, and in terms of industrial availability, a hydrogen atom or a methyl group is more preferable, and a methyl group is still more preferable.

In formula (a0-1), La⁰¹ and La⁰² each independently represents a single bond or a divalent linking group.

The divalent linking group for La⁰¹ and La⁰² is not particularly limited, and preferable examples thereof include a divalent hydrocarbon group which may have a substituent and a divalent linking group containing a hetero atom.

Divalent Hydrocarbon Group which May have a Substituent:

In the case where La⁰¹ and La⁰² is a divalent linking group which may have a substituent, the hydrocarbon group may be either an aliphatic hydrocarbon group or an aromatic hydrocarbon group.

Aliphatic Hydrocarbon Group for La⁰¹ and La⁰²

The “aliphatic hydrocarbon group” refers to a hydrocarbon group that has no aromaticity. The aliphatic hydrocarbon group may be saturated or unsaturated. In general, the aliphatic hydrocarbon group is preferably saturated.

Examples of the aliphatic hydrocarbon group include a linear or branched aliphatic hydrocarbon group, and an aliphatic hydrocarbon group containing a ring in the structure thereof may be given.

Linear or Branched Aliphatic Hydrocarbon Group

The linear aliphatic hydrocarbon group preferably has 1 to 10 carbon atoms, more preferably 1 to 6, still more preferably 1 to 4, and most preferably 1 to 3.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄-] and a pentamethylene group [—(CH₂)₅—].

The branched aliphatic hydrocarbon group preferably has 3 to 10 carbon atoms, more preferably 3 to 6 carbon atoms, still more preferably 3 or 4 carbon atoms, and most preferably 3 carbon atoms.

As the branched aliphatic hydrocarbon group, branched alkylene groups are preferred, and specific examples include various alkylalkylene groups, including alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂—, and —CH₂CH(CH₃)CH₂CH₂—. As the alkyl group within the alkylalkylene group, a linear alkyl group of 1 to 5 carbon atoms is preferable.

The linear or branched aliphatic hydrocarbon group may or may not have a substituent. Examples of the substituent include a fluorine atom, a fluorinated alkyl group of 1 to 5 carbon atoms, and a carbonyl group.

Aliphatic Hydrocarbon Group Containing a Ring in the Structure Thereof

As examples of the hydrocarbon group containing a ring in the structure thereof, a cyclic aliphatic hydrocarbon group containing a hetero atom in the ring structure thereof and may have a substituent (a group in which two hydrogen atoms have been removed from an aliphatic hydrocarbon ring), a group in which the cyclic aliphatic hydrocarbon group is bonded to the terminal of the aforementioned chain-like aliphatic hydrocarbon group, and a group in which the cyclic aliphatic group is interposed within the aforementioned linear or branched aliphatic hydrocarbon group, may be given. As the linear or branched aliphatic hydrocarbon group, the same groups as those described above may be used.

The cyclic aliphatic hydrocarbon group preferably has 3 to 20 carbon atoms, and more preferably 3 to 12 carbon atoms.

The cyclic aliphatic hydrocarbon group may be either a polycyclic group or a monocyclic group. As the monocyclic aliphatic hydrocarbon group, a group in which 2 hydrogen atoms have been removed from a monocycloalkane is preferable. The monocycloalkane preferably has 3 to 6 carbon atoms, and specific examples thereof include cyclopentane and cyclohexane. As the polycyclic group, a group in which two hydrogen atoms have been removed from a polycycloalkane is preferable, and the polycyclic group preferably has 7 to 12 carbon atoms. Examples of the polycycloalkane include adamantane, norbornane, isobornane, tricyclodecane and tetracyclododecane.

The cyclic aliphatic hydrocarbon group may or may not have a substituent. Examples of the substituent include an alkyl group, an alkoxy group, a halogen atom, a halogenated alkyl group, a hydroxyl group and a carbonyl group.

The alkyl group as the substituent is preferably an alkyl group of 1 to 5 carbon atoms, and a methyl group, an ethyl group, a propyl group, an n-butyl group or a tert-butyl group is particularly desirable.

The alkoxy group as the substituent is preferably an alkoxy group having 1 to 5 carbon atoms, more preferably a methoxy group, ethoxy group, n-propoxy group, iso-propoxy group, n-butoxy group or tert-butoxy group, and most preferably a methoxy group or an ethoxy group.

Examples of the halogen atom for the substituent include a fluorine atom, a chlorine atom, a bromine atom and an iodine atom, and a fluorine atom is preferable.

Examples of the halogenated alkyl group for the substituent include groups in which part or all of the hydrogen atoms within the aforementioned alkyl groups has been substituted with the aforementioned halogen atoms.

The cyclic aliphatic hydrocarbon group may have part of the carbon atoms constituting the ring structure thereof substituted with a substituent containing a hetero atom. As the substituent containing a hetero atom, —O—, —C(═O)—O—, —S—, —S(═O)₂— or —S(═O)₂—O— is preferable.

Aromatic Hydrocarbon Group for La⁰¹ and La⁰²

The aromatic hydrocarbon group is a hydrocarbon group having at least one aromatic ring.

The aromatic ring is not particularly limited, as long as it is a cyclic conjugated compound having (4n+2)π (electrons, and may be either monocyclic or polycyclic. The aromatic ring preferably has 5 to 30 carbon atoms, more preferably 5 to 20, still more preferably 6 to 15, and most preferably 6 to 12. Here, the number of carbon atoms within a substituent(s) is not included in the number of carbon atoms of the aromatic hydrocarbon group. Examples of the aromatic ring include aromatic hydrocarbon rings, such as benzene, naphthalene, anthracene and phenanthrene; and aromatic hetero rings in which part of the carbon atoms constituting the aforementioned aromatic hydrocarbon rings has been substituted with a hetero atom. Examples of the hetero atom within the aromatic hetero rings include an oxygen atom, a sulfur atom and a nitrogen atom.

Specific examples of the aromatic hetero ring include a pyridine ring and a thiophene ring.

Specific examples of the aromatic hydrocarbon group include a group in which two hydrogen atoms have been removed from the aforementioned aromatic hydrocarbon ring or aromatic hetero ring (arylene group or heteroarylene group); a group in which two hydrogen atoms have been removed from an aromatic compound having two or more aromatic rings (biphenyl, fluorene or the like); and a group in which one hydrogen atom of the aforementioned aromatic hydrocarbon ring or aromatic hetero ring has been substituted with an alkylene group (a group in which one hydrogen atom has been removed from the aryl group within the aforementioned arylalkyl group such as a benzyl group, a phenethyl group, a 1-naphthylmethyl group, a 2-naphthylmethyl group, a 1-naphthylethyl group, or a 2-naphthylethyl group, or a heteroarylalkyl group). The alkylene group which is bonded to the aforementioned aryl group or heteroaryl group preferably has 1 to 4 carbon atoms, more preferably 1 or 2 carbon atoms, and most preferably 1 carbon atom.

With respect to the aromatic hydrocarbon group, the hydrogen atom within the aromatic hydrocarbon group may be substituted with a substituent. For example, the hydrogen atom bonded to the aromatic ring within the aromatic hydrocarbon group may be substituted with a substituent. Examples of substituents include an alkyl group, an alkoxy group, a halogen atom, a halogenated alkyl group, and a hydroxyl group.

The alkyl group as the substituent is preferably an alkyl group of 1 to 5 carbon atoms, and a methyl group, an ethyl group, a propyl group, an n-butyl group or a tert-butyl group is particularly desirable.

As the alkoxy group, the halogen atom and the halogenated alkyl group for the substituent, the same groups as the aforementioned substituent groups for substituting a hydrogen atom within the cyclic aliphatic hydrocarbon group may be used.

Divalent Linking Group Containing a Hetero Atom

In the case where La⁰¹ and La⁰² is a divalent linking group containing a hetero atom, preferable examples of the linking group include —O—, —C(═O)—O—, —C(═O)—, —O—C(═O)—O—, —C(═O)—NH—, —NH—, —NH—C(═NH)— (H may be substituted with a substituent such as an alkyl group, an acyl group or the like), —S—, —S(═O)₂—, —S(═O)₂—O—, and a group represented by general formula: —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²— or —Y²¹—S(═O)₂—O—Y²²— [in the formulae, Y²¹ and Y²² each independently represents a divalent hydrocarbon group which may have a substituent, O represents an oxygen atom, and m′ represents an integer of 0 to 3].

In the case where the divalent linking group containing a hetero atom is —C(═O)—NH—, —C(═O)—NH—C(═O)—, —NH— or —NH—C(═NH)—, H may be substituted with a substituent such as an alkyl group, an acyl group or the like. The substituent (an alkyl group, an acyl group or the like) preferably has 1 to 10 carbon atoms, more preferably 1 to 8, and most preferably 1 to 5.

In general formulae —Y²¹—O—Y²²—, —Y²¹—O—, —Y²¹C(═O)—O—, —C(═O)—O—Y²¹—, —[Y²¹—C(═O)—O]_{m″}—Y²²—, —Y²¹—O—C(═O)—Y²²— or —Y²¹—S(═O)₂—O—Y²²—, Y²¹ and Y²² each independently represents a divalent hydrocarbon group which may have a substituent. Examples of the divalent hydrocarbon group include the same groups as those described above as the “divalent hydrocarbon group which may have a substituent” in the explanation of the aforementioned divalent linking group.

As Y²¹, a linear aliphatic hydrocarbon group is preferable, more preferably a linear alkylene group, still more preferably a linear alkylene group of 1 to 5 carbon atoms, and a methylene group or an ethylene group is particularly desirable.

As Y²², a linear or branched aliphatic hydrocarbon group is preferable, and a methylene group, an ethylene group or an alkylmethylene group is more preferable. The alkyl group within the alkylmethylene group is preferably a linear alkyl group of 1 to 5 carbon atoms, more preferably a linear alkyl group of 1 to 3 carbon atoms, and most preferably a methyl group.

In the group represented by the formula —[Y²¹—C(═O)—O]_{m″}—Y²²—, m″ represents an integer of 0 to 3, preferably an integer of 0 to 2, more preferably 0 or 1, and most preferably 1. Namely, it is particularly desirable that the group represented by the formula —[Y²¹—C(═O)—O]_{m″}—Y²²— is a group represented by the formula —Y²¹—C(═O)—O—Y²²—. Among these, a group represented by the formula —(CH₂)_{a′}—C(═O)—O—(CH₂)_{b′}— is preferable. In the formula, a′ is an integer of 1 to 10, preferably an integer of 1 to 8, more preferably an integer of 1 to 5, still more preferably 1 or 2, and most preferably 1. b′ is an integer of 1 to 10, preferably an integer of 1 to 8, more preferably an integer of 1 to 5, still more preferably 1 or 2, and most preferably 1.

As La⁰¹, a single bond, an ester bond [—C(═O)—O—], an ether bond (—O—), a linear or branched alkylene group, or a combination of these is preferable.

As La⁰², —O—, —COO—, —CON(R′)—, —OCO—, —CONHCO— or —CONHCS— is preferable. R′ represents a hydrogen atom or a methyl group.

In formula (a0-1), Ya⁰¹ and Ya⁰² each independently represents a single bond or an alkylene group of 1 to 3 carbon atoms. However, the total number of carbon atoms of Ya⁰¹ and Ya⁰² is 3 or less.

Examples of the alkylene group of 1 to 3 carbon atoms for Ya⁰¹ and Ya⁰² include a methylene group [—CH₂—], an ethylene group [—(CH₂)₂—], a trimethylene group [—(CH₂)₃—], —CH(CH₃)—, —CH(CH₂CH₃)—, —C(CH₃)₂—, and —C(CH₃)(CH₂CH₃)—.

As Ya⁰¹ and Ya⁰², a single bond or a methylene group [—CH₂—] is preferable, and a single bond is more preferable.

In formula (a0-1), Ra⁰¹ and Ra⁰² each independently represents a hydrogen atom or an alkyl group of 1 to 4 carbon atoms. Ra⁰¹ and Ra⁰² may be mutually bonded to form a ring; However, the total number of carbon atoms of Ra⁰¹ and Ra⁰² is 2 to 6.

Examples of the alkyl group of 1 to 4 carbon atoms for Ra⁰¹ and Ra⁰² include a methyl group, an ethyl group, a propyl group, an isopropyl group, an n-butyl group, an isobutyl group and a tert-butyl group.

Examples of the ring structure formed by Ra⁰¹ and Ra⁰² include a group in which one hydrogen atom has been removed from a monocycloalkane such as cyclopentane, cyclohexane or cycloheptane.

As Ra⁰¹ and Ra⁰², a hydrogen atom, a methyl group or an ethyl group is preferable, and a methyl group is more preferable.

In formula (a0-1), Ra⁰³ represents a hydrogen atom, an alkyl group, an alkoxy group, a halogen atom, a halogenated alkyl group, a hydroxy group, —COOR″, —OC(═O)R″, a hydroxyalkyl group or a cyano group. R″ represents a hydrogen atom, an alkyl group, a lactone-containing cyclic group, a carbonate-containing cyclic group or an 13 SO₂-containing cyclic group;

The alkyl group for Ra⁰³ is preferably an alkyl group of 1 to 6 carbon atoms. The alkyl group is preferably a linear alkyl group or a branched alkyl group. Specific examples include a methyl group, an ethyl group, a propyl group, an isopropyl group, an n-butyl group, an isobutyl group, a tert-butyl group, a pentyl group, an isopentyl group, a neopentyl group and a hexyl group. Among these, a methyl group or ethyl group is preferable, and a methyl group is most preferable.

The alkoxy group for Ra⁰³ is preferably an alkoxy group of 1 to 6 carbon atoms. The alkoxy group is preferably a linear or branched alkoxy group. Specific examples of the alkoxy groups include the aforementioned alkyl groups for Ra⁰³ having an oxygen atom (—O—) bonded thereto.

Examples of the halogen atom for Ra⁰³ include a fluorine atom, a chlorine atom, a bromine atom and an iodine atom. Among these examples, a fluorine atom is preferable.

Examples of the halogenated alkyl group for Ra⁰³ include groups in which part or all of the hydrogen atoms within the aforementioned alkyl group for Ra⁰³ has been substituted with the aforementioned halogen atoms. As the halogenated alkyl group, a fluorinated alkyl group is preferable, and a perfluoroalkyl group is particularly desirable.

With respect to —COOR″ and —OC(═O)R″ for Ra⁰³, R″ represents a hydrogen atom, an alkyl group, a lactone-containing cyclic group, a carbonate-containing cyclic group or a —SO₂-containing cyclic group.

The alkyl group for R″ may be linear, branched or cyclic, and preferably has 1 to 15 carbon atoms.

When R″ represents a linear or branched alkyl group, it is preferably an alkyl group of 1 to 10 carbon atoms, more preferably an alkyl group of 1 to 5 carbon atoms, and most preferably a methyl group or an ethyl group.

When R″ is a cyclic alkyl group (cycloalkyl group), it preferably has 3 to 15 carbon atoms, more preferably 4 to 12 carbon atoms, and still more preferably 5 to 10 carbon atoms. Specific examples include groups in which one or more hydrogen atoms have been removed from a monocycloalkane or a polycycloalkane such as a bicycloalkane, tricycloalkane or tetracycloalkane which may or may not be substituted with a fluorine atom or a fluorinated alkyl group. Specific examples include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane or tetracyclododecane.

The lactone-containing cyclic group for R″ is the same as defined for the lactone-containing cyclic group described later. Specific examples of the lactone-containing cyclic group include groups represented by general formulae (a2-r-1) to (a2-r-7).

The carbonate-containing cyclic group for R″ is the same as defined for the carbonate-containing cyclic group described later. Specific examples of the carbonate-containing cyclic group include groups represented by general formulae (ax3-r-1) to (ax3-r-3).

The —SO₂-containing cyclic group for R″ is the same as defined for the —SO₂— containing cyclic group described later. Specific examples of the —SO₂-containing cyclic group include groups represented by general formulae (a5-r-1) to (a5-r-4).

The hydroxyalkyl group for Ra⁰³ preferably has 1 to 6 carbon atoms, and specific examples thereof include the alkyl groups for Ra⁰³ in which at least one hydrogen atom has been substituted with a hydroxy group.

In formula (a0-1), n₀₀ represents an integer of 0 to 2, preferably 0 or 1, and more preferably 0. When n₀₀ is 2, the plurality of Ra⁰³ may be the same or different from each other.

In formula (a0-1), A′ represents an alkylene group of 1 to 5 carbon atoms. Examples of the alkylene group of 1 to 5 carbon atoms for A′ include a methylene group, an ethylene group, an n-propylene group, an isopropylene group, an n-butylene group, and an n-pentylene group. Among these examples, as A′, a methylene group is preferable.

In the present embodiment, as the structural unit (a0), a structural unit represented by general formula (a0-11) shown below is preferable.

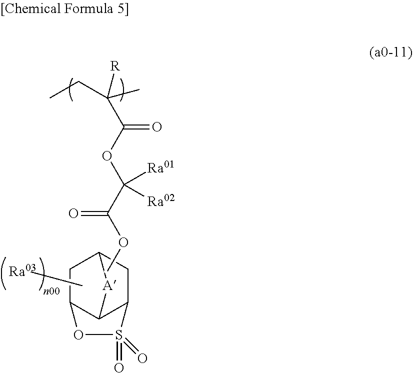

In the formula, R, Ra⁰¹, Ra⁰², Ra⁰³, n₀₀ and A′ are the same as defined for R, Ra⁰¹, Ra⁰², Ra⁰³, n₀₀ and A′ in the aforementioned general formula (a0-1), respectively.

Specific examples of the structural unit (a0) represented by formula (a0-1) are shown below.

In the formulae shown below, R^{α} represents a hydrogen atom, a methyl group or a trifluoromethyl group.

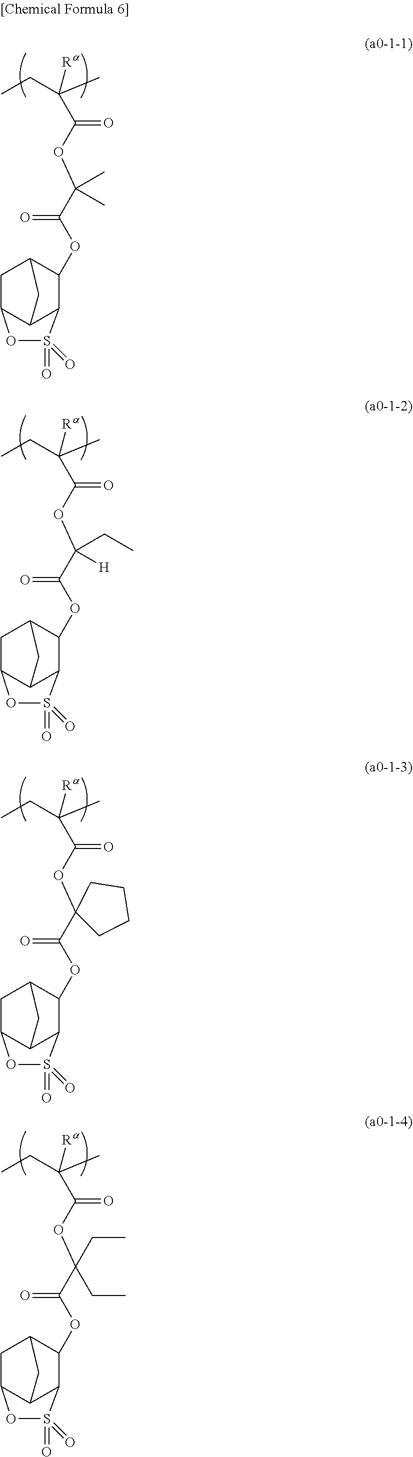

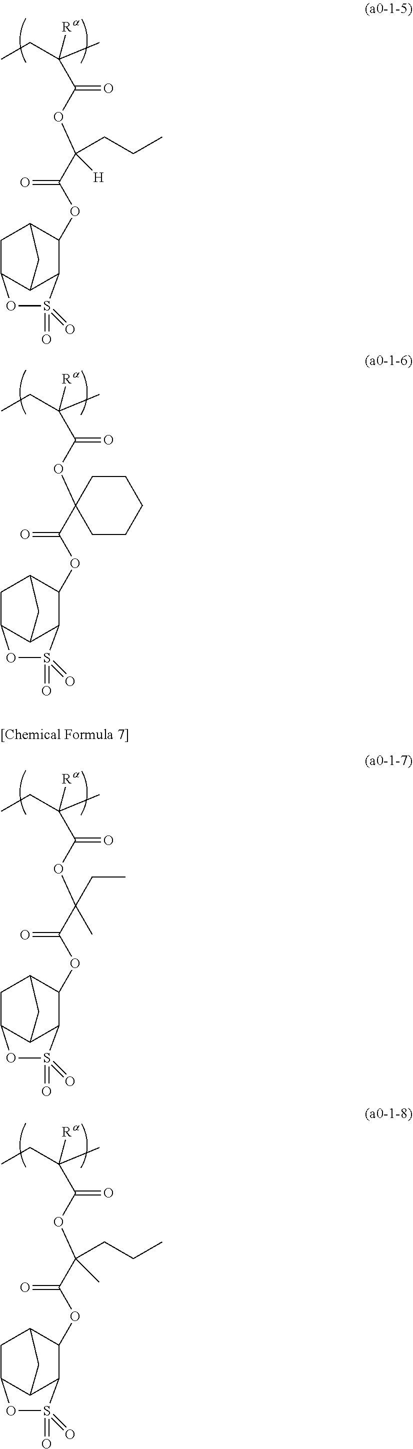

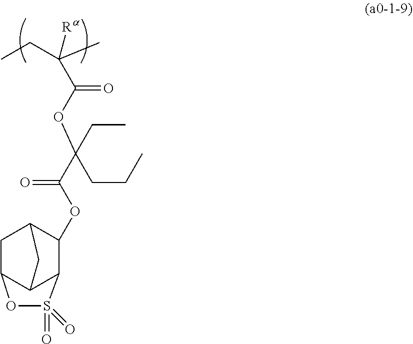

Among these, as the structural unit (a0), at least one member selected from the group consisting of structural units represented by general formulae (a0-1-1) to (a0-1-9) shown below is preferable, at least one member selected from the group consisting of structural units represented by general formulae (a0-1-1) to (a0-1-3) shown below is more preferable, and a structural unit represented by general formula (a0-1-1) is still more preferable.

As the structural unit (a0) contained in the component (A1), 1 kind of structural unit may be used, or 2 or more kinds of structural units may be used.

In the component (A1), the amount of the structural unit (a0) based on the combined total (100 mol %) of all structural units constituting the component (A1) is preferably 5 to 80 mol %, more preferably 10 to 75 mol %, still more preferably 15 to 70 mol %, and most preferably 15 to 60 mol %.

When the amount of the structural unit (a0) is at least as large as the lower limit of the above-mentioned preferable range, the effect of including the structural unit (a0) may be satisfactorily achieved. On the other hand, when the amount of the structural unit (a0) is no more than the upper limit of the above-mentioned preferable range, a good balance may be achieved with the other structural units, and the lithography properties may be improved.

Other Structural Units:

If desired, the component (A1) may include, in addition to the structural unit (a0), other structural unit.

Examples of other structural units include a structural unit (a1) containing an acid decomposable group that exhibits increased polarity by the action of acid; a structural unit (a2) containing a lactone-containing cyclic group or a carbonate-containing cyclic group; a structural unit (a3) containing a polar group-containing aliphatic hydrocarbon group (provided that the structural units (a0), (a1) and (a2) are excluded); a structural unit (a4) containing an acid non-dissociable aliphatic cyclic group; and a structural unit derived from styrene or a derivative thereof.

[Structural Unit (a1)]

The structural unit (a1) is a structural unit containing an acid decomposable group that exhibits increased polarity by the action of acid.

The term “acid decomposable group” refers to a group in which at least a part of the bond within the structure thereof is cleaved by the action of an acid.

Examples of acid decomposable groups which exhibit increased polarity by the action of an acid include groups which are decomposed by the action of an acid to form a polar group.

Examples of the polar group include a carboxy group, a hydroxy group, an amino group and a sulfo group (—SO₃H). Among these, a polar group containing —OH in the structure thereof (hereafter, referred to as “OH-containing polar group”) is preferable, a carboxy group or a hydroxy group is more preferable, and a carboxy group is particularly desirable.

More specifically, as an example of an acid decomposable group, a group in which the aforementioned polar group has been protected with an acid dissociable group (such as a group in which the hydrogen atom of the OH-containing polar group has been protected with an acid dissociable group) may be given.

The “acid dissociable group” refers to both (i) a group in which the bond between the acid dissociable group and the adjacent atom is cleaved by the action of acid; and (ii) a group in which one of the bonds is cleaved by the action of acid, and then a decarboxylation reaction occurs, thereby cleaving the bond between the acid dissociable group and the adjacent atom.

It is necessary that the acid dissociable group that constitutes the acid decomposable group is a group which exhibits a lower polarity than the polar group generated by the dissociation of the acid dissociable group. Thus, when the acid dissociable group is dissociated by the action of acid, a polar group exhibiting a higher polarity than that of the acid dissociable group is generated, thereby increasing the polarity. As a result, the polarity of the entire component (A1) is increased. By the increase in the polarity, the solubility in an alkali developing solution changes, and the solubility in an alkali developing solution is relatively increased, whereas the solubility in an organic developing solution is relatively decreased.

Examples of the acid dissociable group include groups which have been proposed as acid dissociable groups for the base resin of a conventional chemically amplified resist composition.

Specific examples of acid dissociable groups for the base resin of a conventional chemically amplified resist composition include “acetal-type acid dissociable group”, “tertiary alkyl ester-type acid dissociable group” and “tertiary alkyloxycarbonyl acid dissociable group” described below.

Acetal-Type Acid Dissociable Group

Examples of the acid dissociable group for protecting the carboxy group or hydroxy group as a polar group include the acid dissociable group represented by general formula (a1-r-1) shown below (hereafter, referred to as “acetal-type acid dissociable group”).

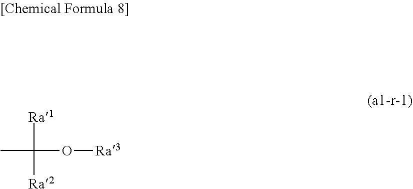

In the formula, Ra¹¹ and Ra¹² represents a hydrogen atom or an alkyl group; and Ra¹³ represents a hydrocarbon group, provided that Ra¹³ may be bonded to Ra¹¹ or Ra¹².

In the formula (a1-r-1), it is preferable that at least one of Ra¹¹ and Ra¹² represents a hydrogen atom, and it is more preferable that both of Ra¹¹ and Ra¹² represent a hydrogen atom.

In the case where Ra¹¹ or Ra¹² is an alkyl group, as the alkyl group, the same alkyl groups as those described above the for the substituent which may be bonded to the carbon atom on the α-position of the aforementioned α-substituted acrylate ester may be mentioned, and an alkyl group of 1 to 5 carbon atoms is preferable. Specific examples include linear or branched alkyl groups. Specific examples of the alkyl group include a methyl group, an ethyl group, a propyl group, an isopropyl group, an n-butyl group, an isobutyl group, a tert-butyl group, a pentyl group, an isopentyl group and a neopentyl group. Of these, a methyl group or an ethyl group is preferable, and a methyl group is particularly preferable.

In formula (a1-r-1), examples of the hydrocarbon group for Ra¹³ include a linear or branched alkyl group and a cyclic hydrocarbon group.

The linear alkyl group preferably has 1 to 5 carbon atoms, more preferably 1 to 4, and still more preferably 1 or 2. Specific examples include a methyl group, an ethyl group, an n-propyl group, an n-butyl group and an n-pentyl group. Among these, a methyl group, an ethyl group or an n-butyl group is preferable, and a methyl group or an ethyl group is more preferable.

The branched alkyl group preferably has 3 to 10 carbon atoms, and more preferably 3 to 5. Specific examples include an isopropyl group, an isobutyl group, a tert-butyl group, an isopentyl group, a neopentyl group a 1,1-diethylpropyl group and a 2,2-dimethylbutyl group. Among these, an isopropyl group is preferable.

In the case where Ra¹³ represents a cyclic hydrocarbon group, the cyclic hydrocarbon group may be an aliphatic hydrocarbon group or an aromatic hydrocarbon group, and may be polycyclic or monocyclic.

As the monocyclic aliphatic hydrocarbon group, a group in which 1 hydrogen atom has been removed from a monocycloalkane is preferable. The monocycloalkane preferably has 3 to 6 carbon atoms, and specific examples thereof include cyclopentane and cyclohexane.

As the polycyclic aliphatic hydrocarbon group, a group in which 1 hydrogen atom has been removed from a polycycloalkane is preferable, and the polycyclic group preferably has 7 to 12 carbon atoms. Examples of the polycycloalkane include adamantane, norbornane, isobornane, tricyclodecane and tetracyclododecane.

When the monovalent hydrocarbon group for Ra¹³ is an aromatic hydrocarbon group, the aromatic hydrocarbon group is a hydrocarbon group having at least one aromatic ring.

The aromatic ring is not particularly limited, as long as it is a cyclic conjugated compound having (4n+2)π (electrons, and may be either monocyclic or polycyclic. The aromatic ring preferably has 5 to 30 carbon atoms, more preferably 5 to 20, still more preferably 6 to 15, and most preferably 6 to 12.

Examples of the aromatic ring include aromatic hydrocarbon rings, such as benzene, naphthalene, anthracene and phenanthrene; and aromatic hetero rings in which part of the carbon atoms constituting the aforementioned aromatic hydrocarbon rings has been substituted with a hetero atom. Examples of the hetero atom within the aromatic hetero rings include an oxygen atom, a sulfur atom and a nitrogen atom. Specific examples of the aromatic hetero ring include a pyridine ring and a thiophene ring.

Specific examples of the aromatic hydrocarbon group for Ra¹³ include a group in which one hydrogen atom has been removed from the aforementioned aromatic hydrocarbon ring or aromatic hetero ring (aryl group or heteroaryl group); a group in which one hydrogen atom has been removed from an aromatic compound having two or more aromatic rings (biphenyl, fluorene or the like); and a group in which one hydrogen atom of the aforementioned aromatic hydrocarbon ring or aromatic hetero ring has been substituted with an alkylene group (an arylalkyl group such as a benzyl group, a phenethyl group, a 1-naphthylmethyl group, a 2-naphthylmethyl group, a 1-naphthylethyl group, or a 2-naphthylethyl group). The alkylene group bonded to the aforementioned aromatic hydrocarbon ring or the aromatic hetero ring preferably has 1 to 4 carbon atoms, more preferably 1 or 2 carbon atoms, and most preferably 1 carbon atom.

In the case where Ra¹³ is bonded to Ra¹¹ or Ra¹² to form a ring, the cyclic group is preferably a 4 to 7-membered ring, and more preferably a 4 to 6-membered ring. Specific examples of the cyclic group include tetrahydropyranyl group and tetrahydrofuranyl group.

In formula (a1-r-1), the hydrocarbon group for Ra¹³ is preferably an alicyclic hydrocarbon group, more preferably a polycyclic aliphatic hydrocarbon group, still more preferably a group in which one hydrogen atom has been removed from adamantane, norbornane, isobornane, tricyclodecane or tetracyclododecane, and most preferably a group in which one hydrogen atom has been removed from adamantane.

Tertiary Alkyl Ester-Type Acid Dissociable Group

Examples of the acid dissociable group for protecting the carboxy group as a polar group include the acid dissociable group represented by general formula (a1-r-2) shown below.

Among the acid dissociable groups represented by general formula (a1-r-2), for convenience, a group which is constituted of alkyl groups is referred to as “tertiary ester-type acid dissociable group”.

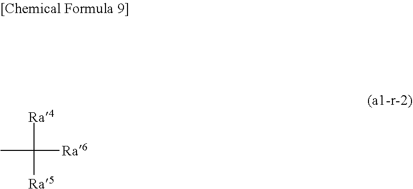

In the formula, Ra¹⁴ to Ra¹⁶ each independently represents a hydrocarbon group, provided that Ra¹⁵ and Ra¹⁶ may be mutually bonded to form a ring.

As the hydrocarbon group for Ra¹⁴ to Ra¹⁶, the same groups as those described above for Ra¹³ may be mentioned.

Ra¹⁴ is preferably an alkyl group of 1 to 5 carbon atoms. In the case where Ra¹⁵ and Ra¹⁶ are mutually bonded to form a ring, a group represented by general formula (a1-r2-1) shown below may be mentioned. On the other hand, in the case where Ra¹⁴ to Ra¹⁶ are not mutually bonded and independently represent a hydrocarbon group, the group represented by general formula (a1-r2-2) shown below may be mentioned.

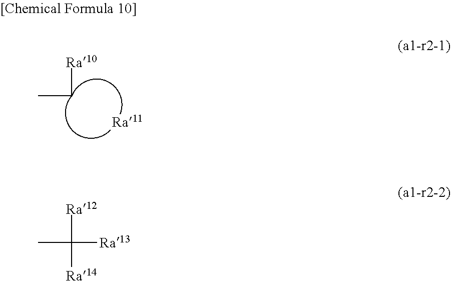

In the formulae, Ra¹¹⁰ represents an alkyl group of 1 to 10 carbon atoms; Ra¹¹¹ is a group which forms an aliphatic cyclic group together with a carbon atom having Ra¹¹⁰ bonded thereto; and Ra¹¹² to Ra¹¹⁴ each independently represents a hydrocarbon group.

In the formula (a1-r2-1), as the alkyl group of 1 to 10 carbon atoms for Ra¹¹⁰, the same groups as described above for the linear or branched alkyl group for Ra¹³ in the formula (a1-r-1) are preferable, a branched alkyl group is more preferable, an isobutyl group or a tert-butyl group is still more preferable, and a tert-butyl group is most preferable. In formula (a1-r2-1), the aliphatic cyclic group formed by Ra¹¹¹ together with the carbon atom to which Ra¹¹⁰ is bonded is preferably a monocyclic or polycyclic aliphatic hydrocarbon group described above for Ra¹³ in formula (a1-r-1), more preferably a monocyclic aliphatic hydrocarbon group, still more preferably a group in which one hydrogen atom has been removed from cyclopentane or cyclohexane, and most preferably a group in which one hydrogen atom has been removed from cyclopentane.

In the formula (a1-r2-2), it is preferable that Ra¹¹² and Ra¹¹⁴ each independently represents an alkyl group or 1 to 10 carbon atoms, and it is more preferable that the alkyl group is the same group as the described above for the linear or branched alkyl group for Ra¹³ in the formula (a1-r-1), it is still more preferable that the alkyl group is a linear alkyl group of 1 to 5 carbon atoms, and it is particularly preferable that the alkyl group is a methyl group or an ethyl group.

In the formula (a1-r2-2), it is preferable that Ra¹¹³ is the same group as described above for the linear or branched alkyl group or monocyclic or polycyclic alicyclic hydrocarbon group for Ra¹³ in the formula (a1-r-1). Among these examples, a monocyclic or polycyclic aliphatic hydrocarbon group described above for Ra¹³ is preferable, a polycyclic aliphatic hydrocarbon group is more preferable, a group in which one hydrogen atom has been removed from adamantane, norbornane, isobornane, tricyclodecane or tetracyclododecane is more preferable, and a group in which one hydrogen atom has been removed from adamantane is still more preferable.

Specific examples of the group represented by the aforementioned formula (a1-r2-1) are shown below. * represents a valence bond (the same applies throughout the present specification).

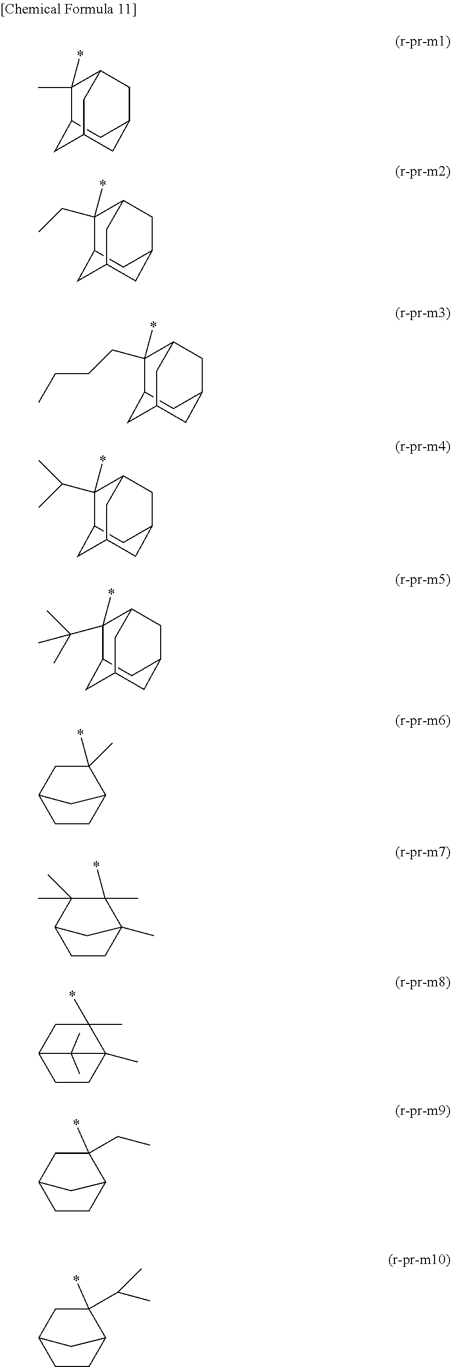

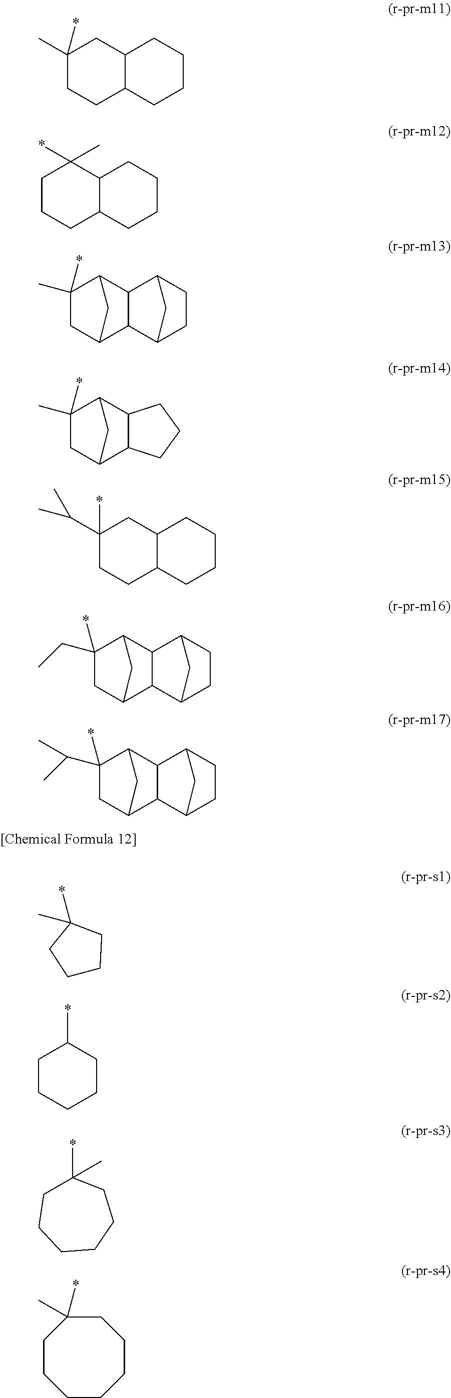

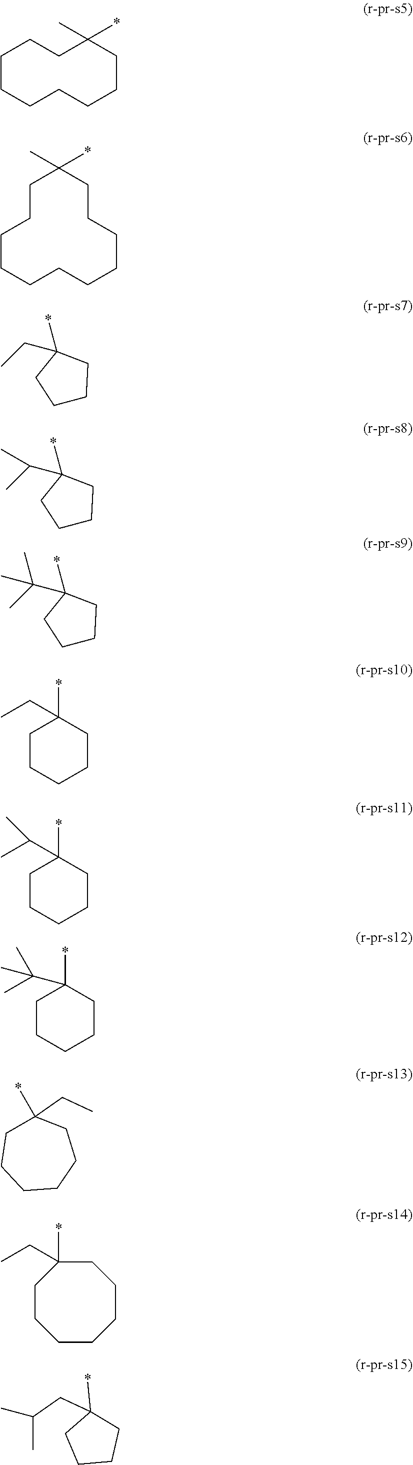

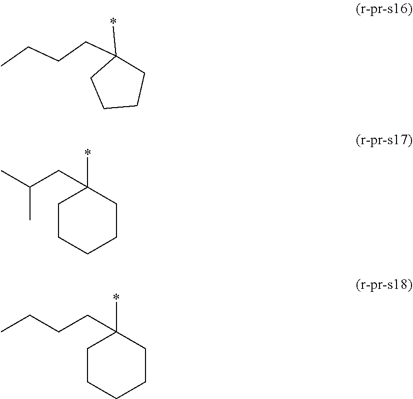

Specific examples of the group represented by the aforementioned formula (a1-r2-2) are shown below.

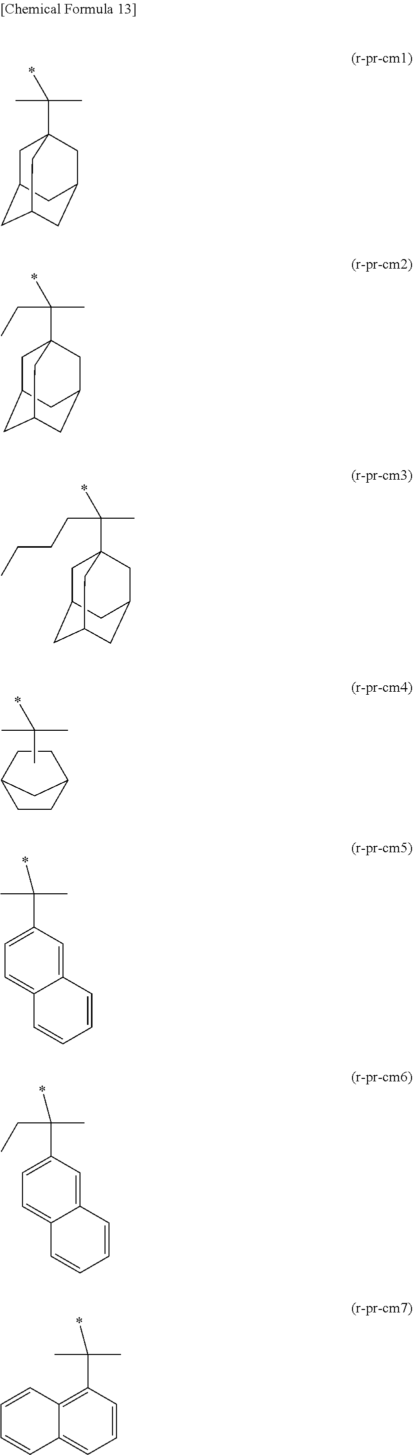

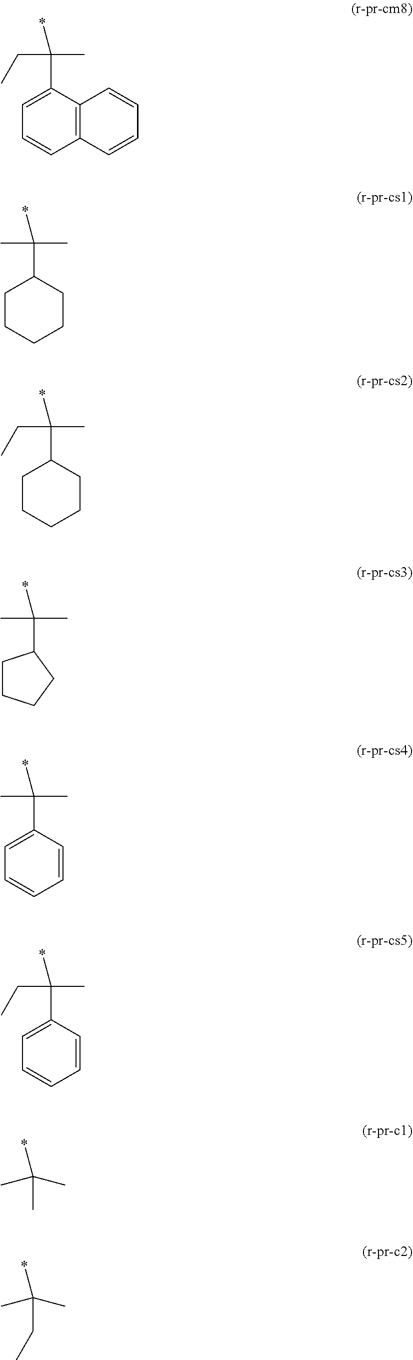

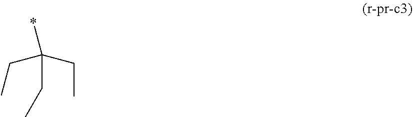 [SMILES: *C(CC)(CC)CC]

Tertiary Alkyloxycarbonyl Acid Dissociable Group

Examples of the acid dissociable group for protecting a hydroxy group as a polar group include the acid dissociable group represented by general formula (a1-r-3) shown below (hereafter, for convenience, referred to as “tertiary alkyloxycarbonyl-type acid dissociable group”).

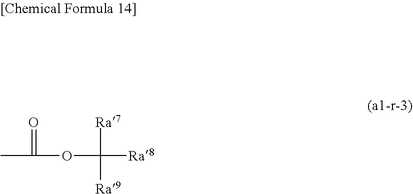

In the formula, Ra¹⁷ to Ra¹⁹ each independently represents an alkyl group.

In the formula (a1-r-3), Ra′⁷ to Ra¹⁹ is preferably an alkyl group of 1 to 5 carbon atoms, and more preferably an alkyl group of 1 to 3 carbon atoms.

Further, the total number of carbon atoms within the alkyl group is preferably 3 to 7, more preferably 3 to 5, and most preferably 3 or 4.

Examples of the structural unit (a1) include a structural unit derived from an acrylate ester which may have the hydrogen atom bonded to the carbon atom on the α-position substituted with a substituent; a structural unit derived from an acrylamide; a structural unit derived from hydroxystyrene or a hydroxystyrene derivative in which at least a part of the hydrogen atom of the hydroxy group is protected with an acid dissociable group; and a structural unit derived from vinylbenzoic acid or a vinylbenzoic acid derivative in which at least a part of the hydrogen atom within —C(═O)—OH is protected with an acid dissociable group.

As the structural unit (a1), a structural unit derived from an acrylate ester which may have the hydrogen atom bonded to the carbon atom on the α-position substituted with a substituent is preferable.

Specific examples of preferable structural units for the structural unit (a1) include structural units represented by general formula (a1-1) or (a1-2) shown below.

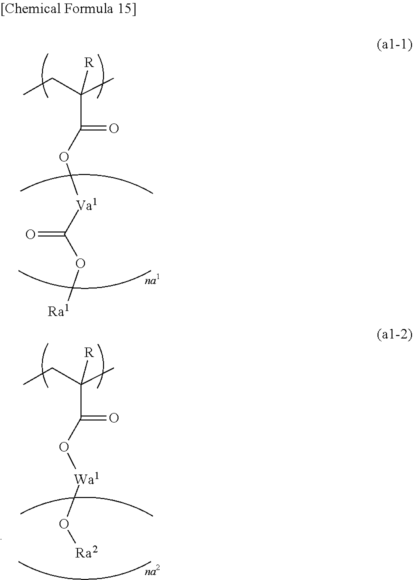

In the formula, R represents a hydrogen atom, an alkyl group of 1 to 5 carbon atoms or a halogenated alkyl group of 1 to 5 carbon atoms; Va¹ represents a divalent hydrocarbon group which may contain an ether bond; nₐ₁ represents an integer of 0 to 2; and Ra¹ represents an acid dissociable group represented by the aforementioned formula (a1-r-1) or (a1-r-2). Wa¹ represents a hydrocarbon group having a valency of nₐ₂+1; nₐ₂, represents an integer of 1 to 3; and Ra² represents an acid dissociable group represented by the aforementioned formula (a1-r-1) or (a1-r-3).

In the aforementioned formula (a1-1), as the alkyl group of 1 to 5 carbon atoms for R, a linear or branched alkyl group of 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group and a neopentyl group. The halogenated alkyl group of 1 to 5 carbon atoms represented by R is a group in which part or all of the hydrogen atoms of the aforementioned alkyl group of 1 to 5 carbon atoms have been substituted with halogen atoms. Examples of the halogen atom include a fluorine atom, a chlorine atom, a bromine atom and an iodine atom, and a fluorine atom is particularly desirable.

As R, a hydrogen atom, an alkyl group of 1 to 5 carbon atoms or a fluorinated alkyl group of 1 to 5 carbon atoms is preferable, and a hydrogen atom or a methyl group is particularly desirable in terms of industrial availability.

In formula (a1-1), the divalent hydrocarbon group for V¹ may be either an aliphatic hydrocarbon group or an aromatic hydrocarbon group.

The aliphatic hydrocarbon group as the divalent hydrocarbon group for Va¹ may be either saturated or unsaturated. In general, the aliphatic hydrocarbon group is preferably saturated.

As specific examples of the aliphatic hydrocarbon group, a linear or branched aliphatic hydrocarbon group, and an aliphatic hydrocarbon group containing a ring in the structure thereof may be given.

The linear or branched aliphatic hydrocarbon group preferably has 1 to 10 carbon atoms, more preferably 1 to 6, still more preferably 1 to 4, and most preferably 1 to 3.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄-] and a pentamethylene group [—(CH₂)₅—].

As the branched aliphatic hydrocarbon group, branched alkylene groups are preferred, and specific examples include various alkylalkylene groups, including alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂—, and —CH₂CH(CH₃)CH₂CH₂—. As the alkyl group within the alkylalkylene group, a linear alkyl group of 1 to 5 carbon atoms is preferable.

As examples of the hydrocarbon group containing a ring in the structure thereof, an alicyclic hydrocarbon group (a group in which two hydrogen atoms have been removed from an aliphatic hydrocarbon ring), a group in which the alicyclic hydrocarbon group is bonded to the terminal of the aforementioned chain-like aliphatic hydrocarbon group, and a group in which the alicyclic group is interposed within the aforementioned linear or branched aliphatic hydrocarbon group, may be given. The linear or branched aliphatic hydrocarbon group is the same as defined for the aforementioned linear aliphatic hydrocarbon group or the aforementioned branched aliphatic hydrocarbon group.

The alicyclic hydrocarbon group preferably has 3 to 20 carbon atoms, and more preferably 3 to 12 carbon atoms.

The alicyclic hydrocarbon group may be either a monocyclic group or a polycyclic group. As the monocyclic aliphatic hydrocarbon group, a group in which 2 hydrogen atoms have been removed from a monocycloalkane is preferable. The monocycloalkane preferably has 3 to 6 carbon atoms, and specific examples thereof include cyclopentane and cyclohexane. As the polycyclic group, a group in which two hydrogen atoms have been removed from a polycycloalkane is preferable, and the polycyclic group preferably has 7 to 12 carbon atoms. Examples of the polycycloalkane include adamantane, norbornane, isobornane, tricyclodecane and tetracyclododecane.

The aromatic hydrocarbon group as the divalent hydrocarbon group for Va¹ is a hydrocarbon group having an aromatic ring.

The aromatic hydrocarbon group preferably has 3 to 30 carbon atoms, more preferably 5 to 30, still more preferably 5 to 20, still more preferably 6 to 15, and most preferably 6 to 10. Here, the number of carbon atoms within a substituent(s) is not included in the number of carbon atoms of the aromatic hydrocarbon group.

Examples of the aromatic ring contained in the aromatic hydrocarbon group include aromatic hydrocarbon rings, such as benzene, biphenyl, fluorene, naphthalene, anthracene and phenanthrene; and aromatic hetero rings in which part of the carbon atoms constituting the aforementioned aromatic hydrocarbon rings has been substituted with a hetero atom. Examples of the hetero atom within the aromatic hetero rings include an oxygen atom, a sulfur atom and a nitrogen atom.

Specific examples of the aromatic hydrocarbon group include a group in which two hydrogen atoms have been removed from the aforementioned aromatic hydrocarbon ring (arylene group); and a group in which one hydrogen atom has been removed from the aforementioned aromatic hydrocarbon ring (aryl group) and one hydrogen atom has been substituted with an alkylene group (such as a benzyl group, a phenethyl group, a 1-naphthylmethyl group, a 2-naphthylmethyl group, a 1-naphthylethyl group, or a 2-naphthylethyl group). The alkylene group (alkyl chain within the arylalkyl group) preferably has 1 to 4 carbon atom, more preferably 1 or 2, and most preferably 1.

In the aforementioned formula (a1-2), the hydrocarbon group for Wa¹ having a valency of nₐ₂+1 may be either an aliphatic hydrocarbon group or an aromatic hydrocarbon group. The aliphatic cyclic group refers to a hydrocarbon group that has no aromaticity, and may be either saturated or unsaturated, but is preferably saturated. Examples of the aliphatic hydrocarbon group include a linear or branched aliphatic hydrocarbon group, an aliphatic hydrocarbon group containing a ring in the structure thereof, and a combination of the linear or branched aliphatic hydrocarbon group and the aliphatic hydrocarbon group containing a ring in the structure thereof.

The valency of nₐ₂+1 is preferably divalent, trivalent or tetravalent, and divalent or trivalent is more preferable.

Specific examples of structural unit represented by formula (a1-1) are shown below. In the formulae shown below, Rα represents a hydrogen atom, a methyl group or a trifluoromethyl group.

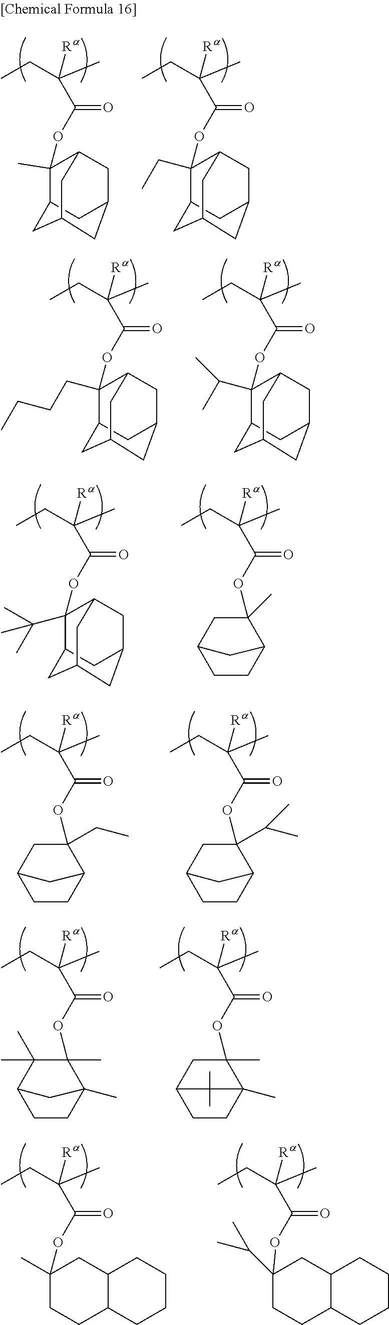

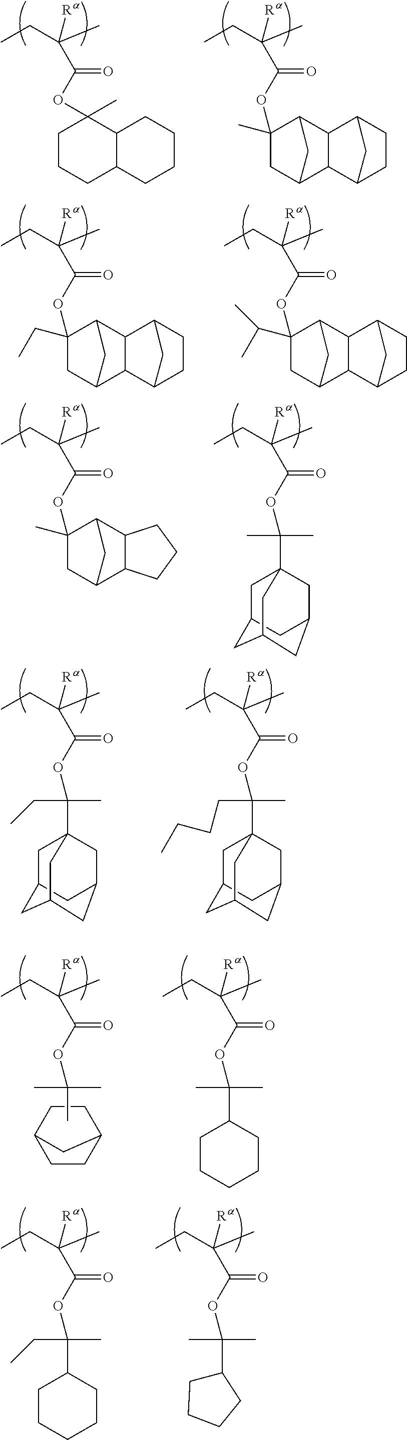

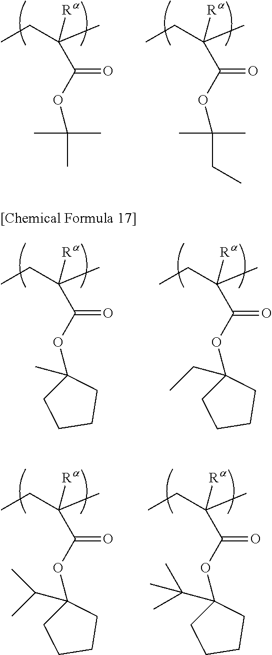

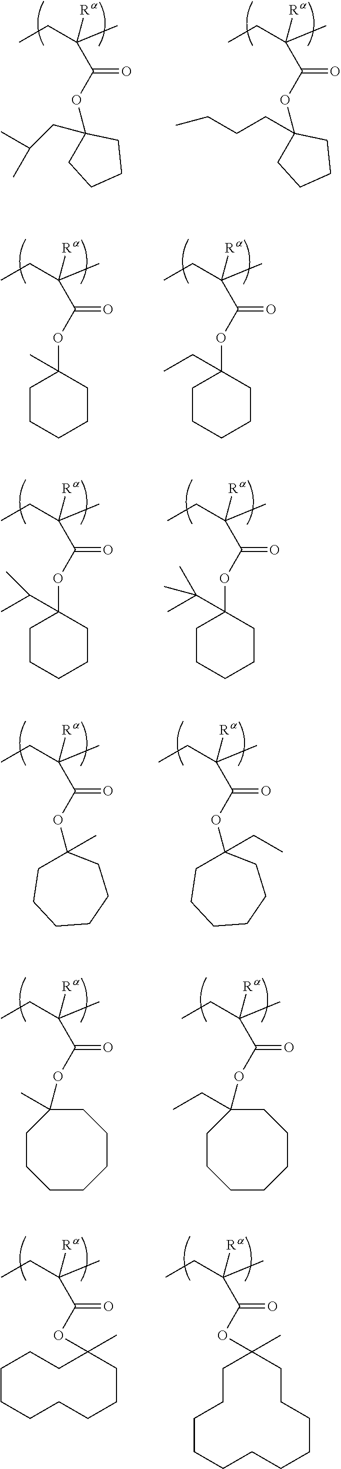

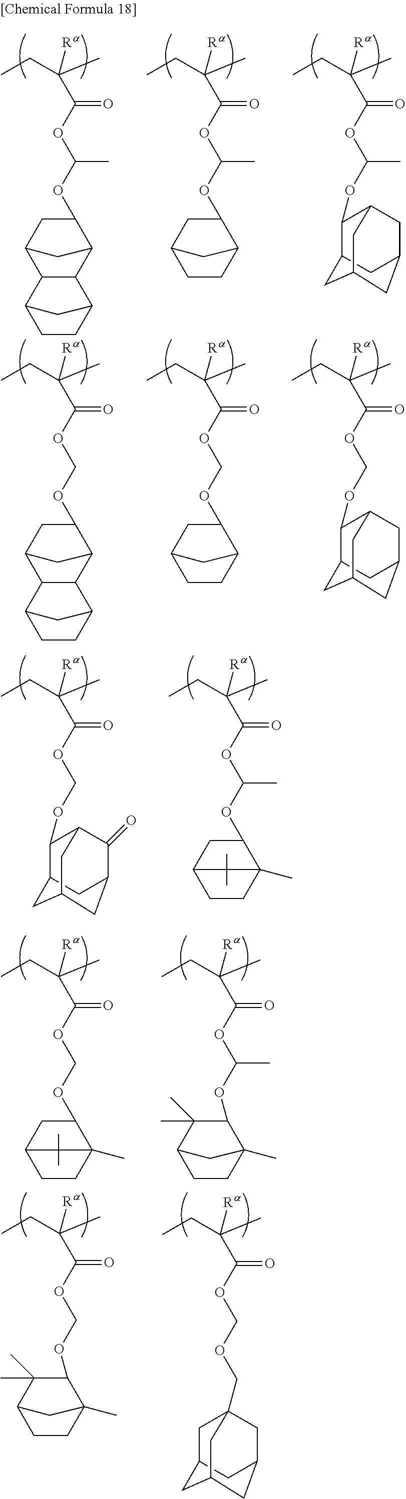

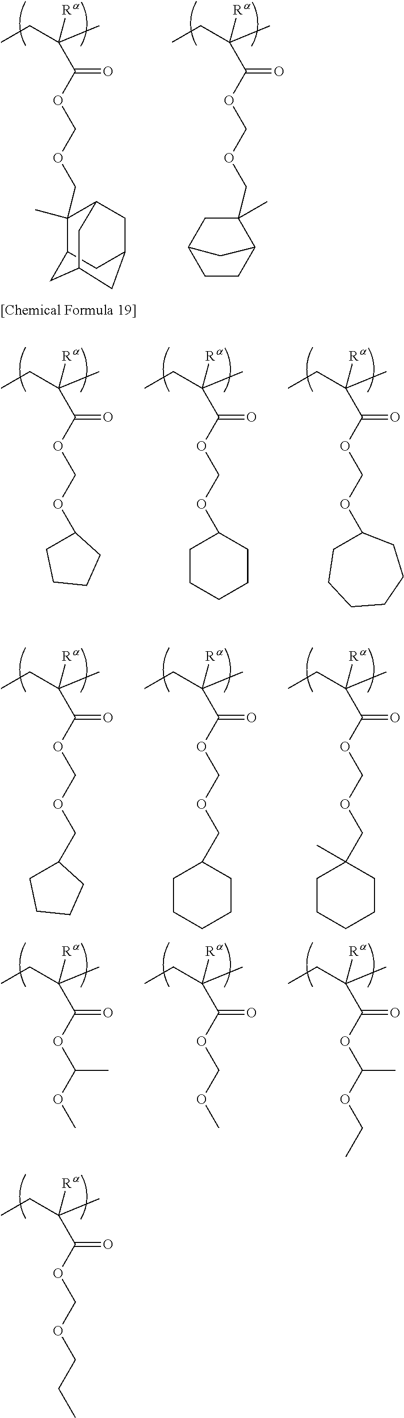

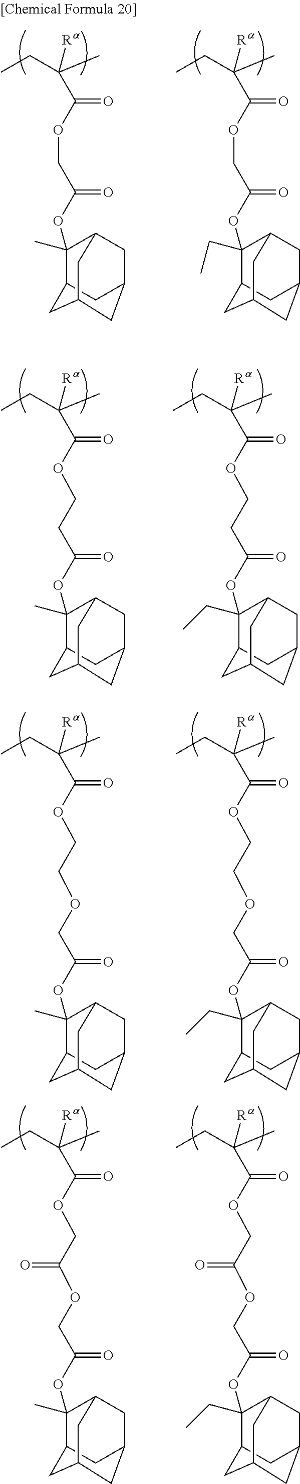

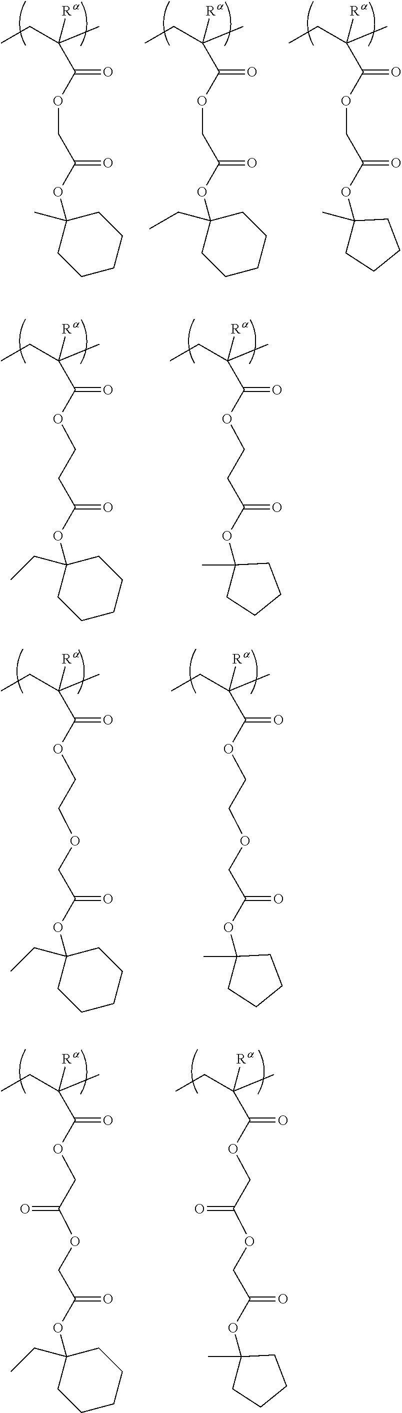

Specific examples of structural unit represented by formula (a1-2) are shown below.

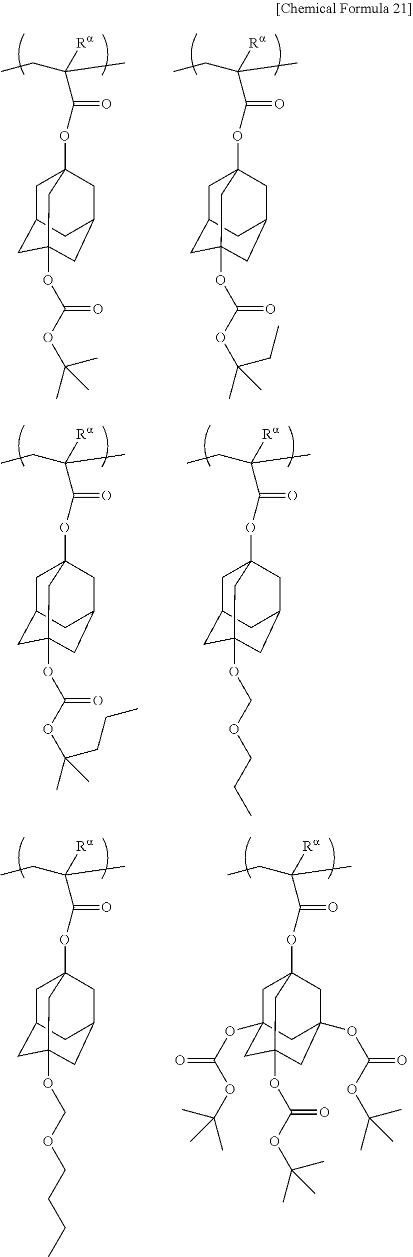

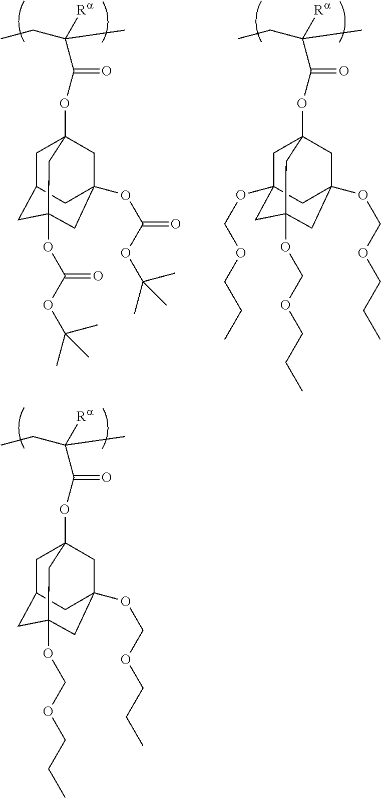

As the structural unit (a1) contained in the component (A1), 1 type of structural unit may be used, or 2 or more types may be used.

In the component (A1), the amount of the structural unit (a1) based on the combined total of all structural units constituting the component (A1) is preferably 5 to 70 mol %, more preferably 10 to 65 mol %, and still more preferably 20 to 60 mol %.

When the amount of the structural unit (a1) is at least as large as the lower limit of the above-mentioned range, a resist pattern may be reliably obtained, and the sensitivity, resolution, roughness and various lithography properties such as EL margin are further improved. On the other hand, when the amount of the structural unit (a1) is no more than the upper limit of the above-mentioned range, a good balance may be achieved with the other structural units.

[Structural Unit (a2)]

The component (A1) may include, in addition to the structural unit (a0), a structural unit (a2) containing a lactone-containing cyclic group or a carbonate-containing cyclic group.

When the component (A1) is used for forming a resist film, the structural unit (a2) containing a lactone-containing cyclic group or a carbonate-containing cyclic group is effective in improving the adhesion between the resist film and the substrate. Further, by virtue of including the structural unit (a2), in an alkali developing process, during developing, the solubility of the resist film in an alkali developing is enhanced.

The term “lactone-containing cyclic group” refers to a cyclic group including a ring containing a —O—C(═O)— structure (lactone ring). The term “lactone ring” refers to a single ring containing a —O—C(O)— structure, and this ring is counted as the first ring. A lactone-containing cyclic group in which the only ring structure is the lactone ring is referred to as a monocyclic group, and groups containing other ring structures are described as polycyclic groups regardless of the structure of the other rings. The lactone-containing cyclic group may be either a monocyclic group or a polycyclic group.

The lactone-containing cyclic group for the structural unit (a2) is not particularly limited, and an arbitrary structural unit may be used. Specific examples include groups represented by general formulae (a2-r-1) to (a2-r-7) shown below.

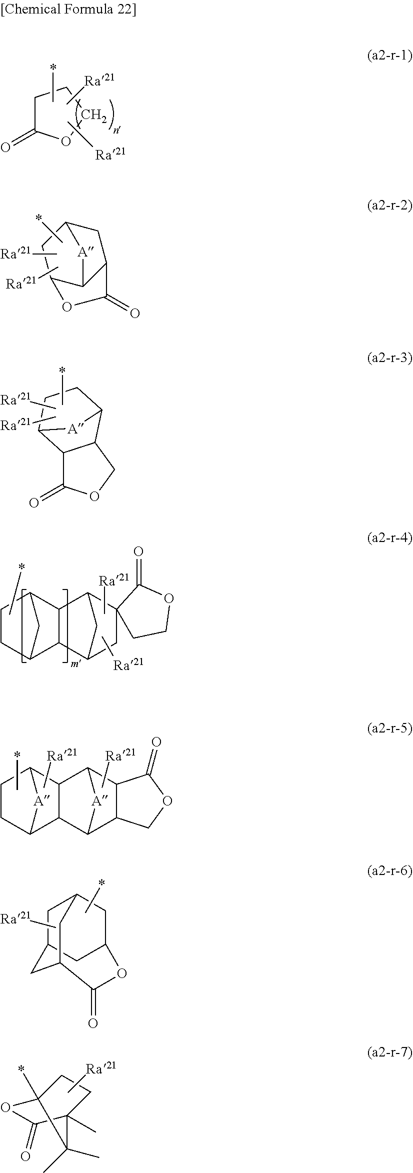

In the formulae, each Ra¹²¹ independently represents a hydrogen atom, an alkyl group, an alkoxy group, a halogen atom, a halogenated alkyl group, a hydroxy group, —COOR″, —OC(═O)R″, a hydroxyalkyl group or a cyano group; R″ represents a hydrogen atom, an alkyl group, a lactone-containing cyclic group, a carbonate-containing cyclic group or an —SO₂-containing cyclic group; A″ represents an oxygen atom, a sulfur atom, or an alkylene group of 1 to 5 carbon atoms which may contain an oxygen atom or a sulfur atom; n′ represents an integer of 0 to 2; and m′ represents 0 or 1.

In formulae (a2-r-1) to (a2-r-7), examples of the alkyl group, alkoxy group, halogen atom, halogenated alkyl group, —COOR″, —OC(═O)R″ and hydroxyalkyl group for Ra¹²¹ include the same groups as those described above in the explanation of Ra⁰³ in the general formula (a0-1).

In formulae (a2-r-2), (a2-r-3) and (a2-r-5), as the alkylene group of 1 to 5 carbon atoms represented by A″, a linear or branched alkylene group is preferable, and examples thereof include a methylene group, an ethylene group, an n-propylene group and an isopropylene group. Examples of alkylene groups that contain an oxygen atom or a sulfur atom include the aforementioned alkylene groups in which —O— or —S— is bonded to the terminal of the alkylene group or present between the carbon atoms of the alkylene group. Specific examples of such alkylene groups include —O—CH₂—, —CH₂—O—CH₂—, —S—CH₂—, and —CH₂—S—CH₂—. As A″, an alkylene group of 1 to 5 carbon atoms or —O— is preferable, more preferably an alkylene group of 1 to 5 carbon atoms, and most preferably a methylene group.

Specific examples of the groups represented by the aforementioned general formulae (a2-r-1) to (a2-r-7) are shown below.

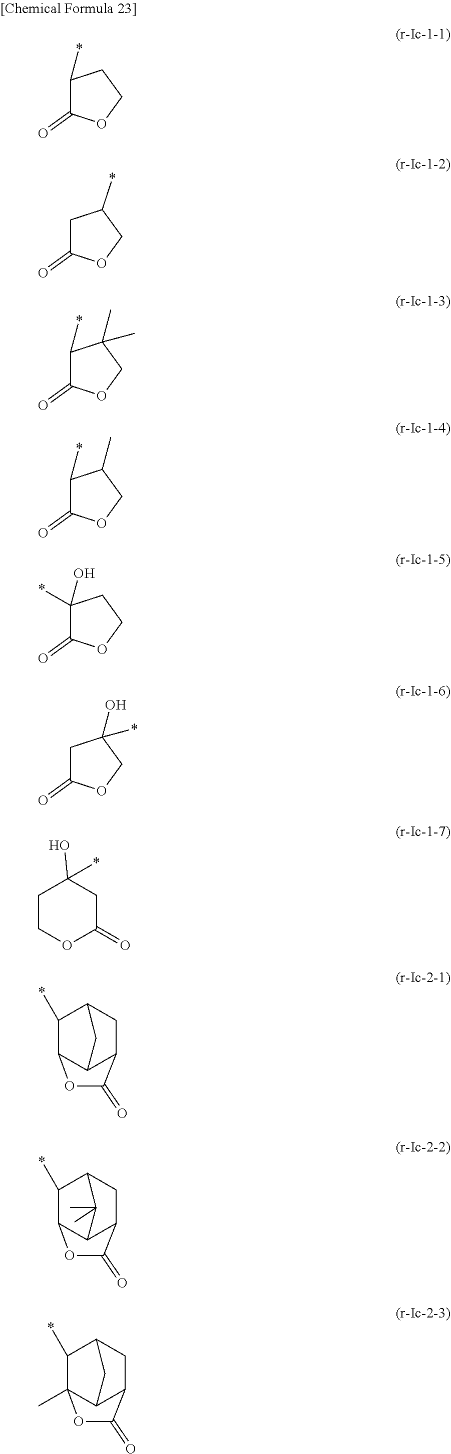

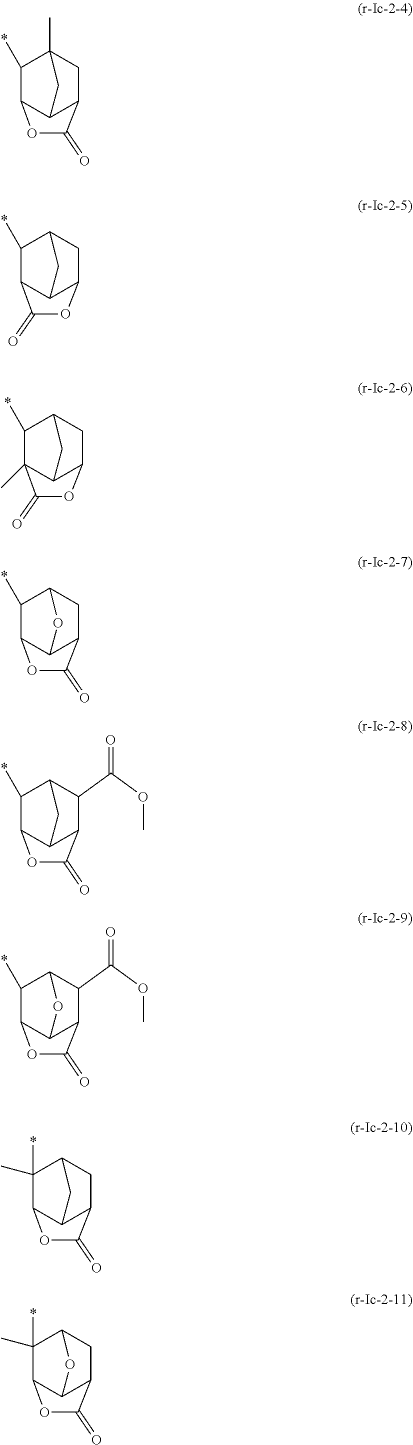

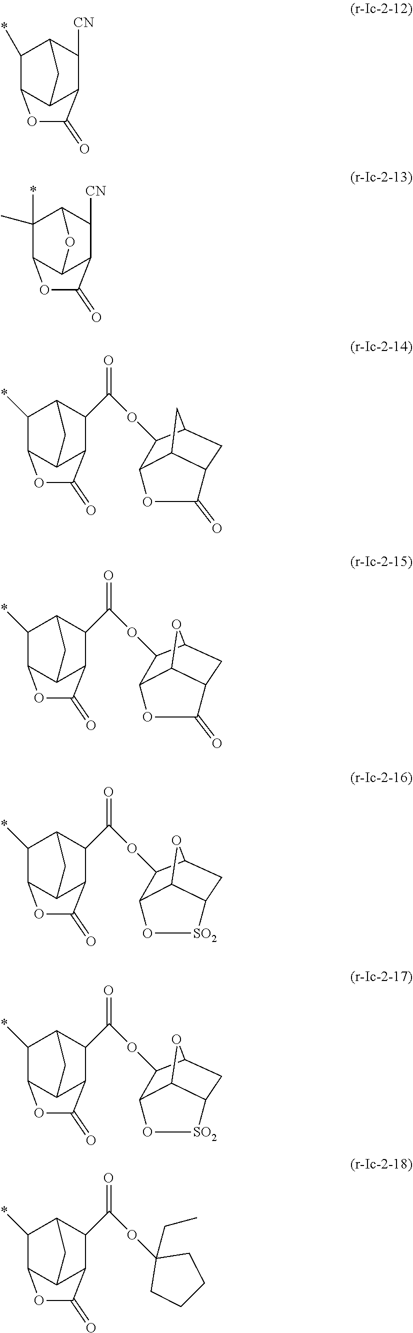

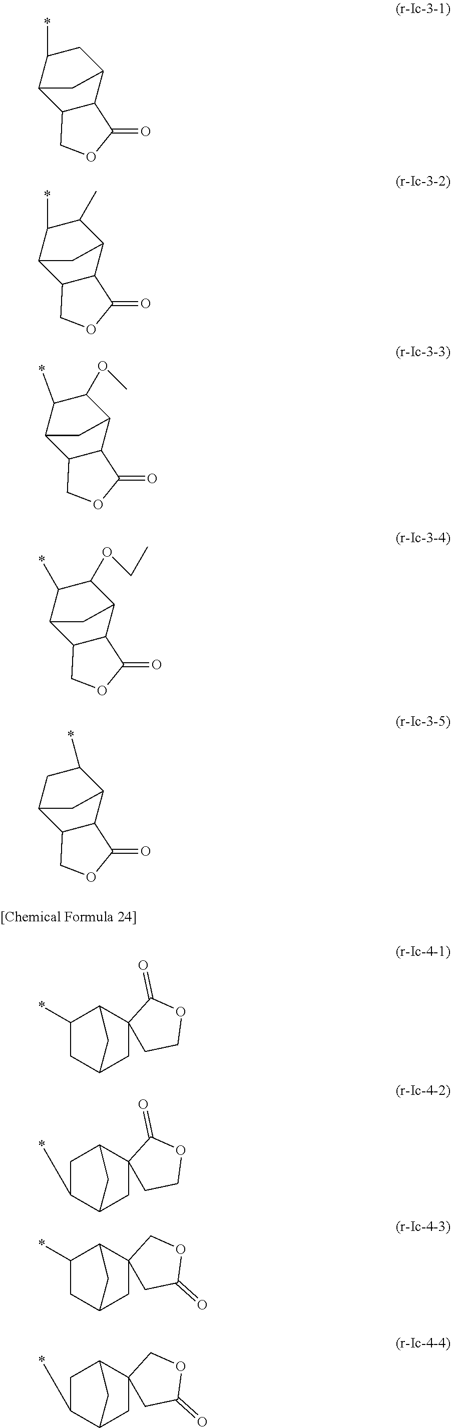

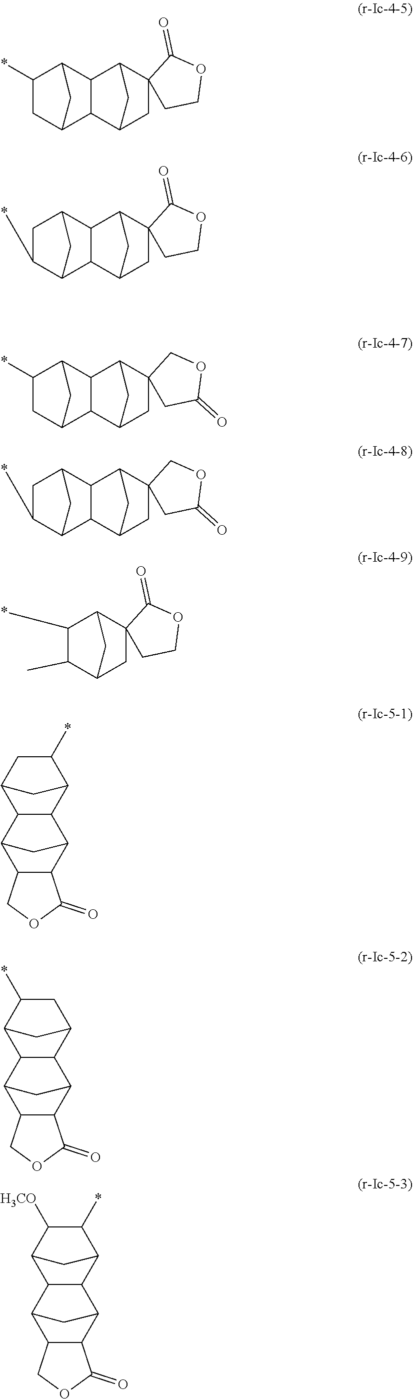

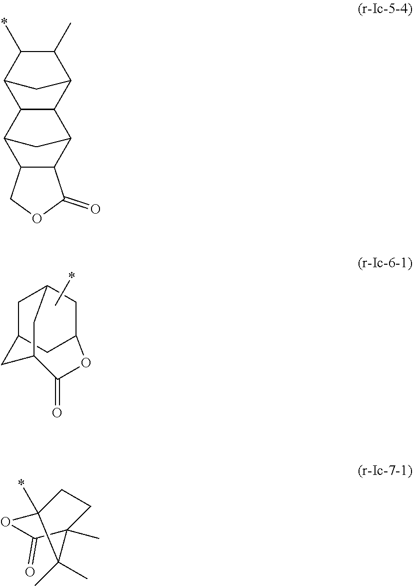

The term “carbonate-containing cyclic group” refers to a cyclic group including a ring containing a —O—C(═O)—O— structure (carbonate ring). The term “carbonate ring” refers to a single ring containing a —O—C(═O)—O— structure, and this ring is counted as the first ring. A carbonate-containing cyclic group in which the only ring structure is the carbonate ring is referred to as a monocyclic group, and groups containing other ring structures are described as polycyclic groups regardless of the structure of the other rings. The carbonate-containing cyclic group may be either a monocyclic group or a polycyclic group.

The carbonate-containing cyclic group is not particularly limited, and an arbitrary group may be used. Specific examples include groups represented by general formulae (ax3-r-1) to (ax3-r-3) shown below.

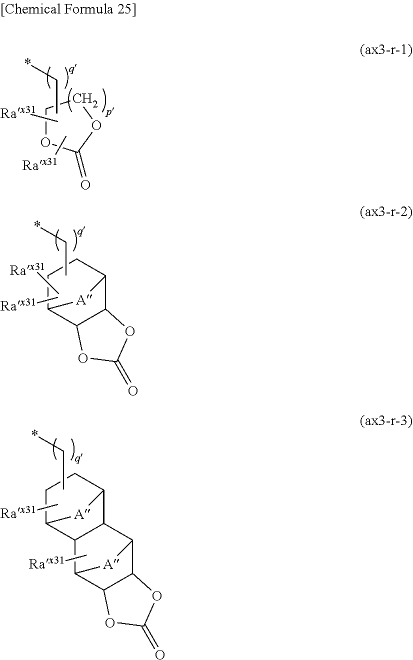

In the formulae, each Ra″ independently represents an alkyl group, an alkoxy group, a halogen atom, a halogenated alkyl group, —COOR″, —OC(═O)R″, a hydroxyalkyl group or a cyano group; R″ represents a hydrogen atom, an alkyl group, a lactone-containing cyclic group, a carbonate-containing cyclic group or an —SO₂-containing cyclic group; A″ represents an oxygen atom, a sulfur atom, or an alkylene group of 1 to 5 carbon atoms with or without an oxygen atom or a sulfur atom; p′ represents an integer of 0 to 3; and q′ is 0 or 1.

In general formulae (ax3-r-2) and (ax3-r-3), A″ is the same as defined for A″ in general formulae (a2-r-2), (a2-r-3) and (a2-r-5).

Examples of the alkyl group, alkoxy group, halogen atom, halogenated alkyl group, —COOR″, —OC(═O)R″ and hydroxyalkyl group for Ra¹³¹ include the same groups as those described above in the explanation of Ra⁰³ in the aforementioned general formula (a0-1).

Specific examples of the groups represented by the aforementioned general formulae (ax3-r-1) to (ax3-r-3) are shown below.

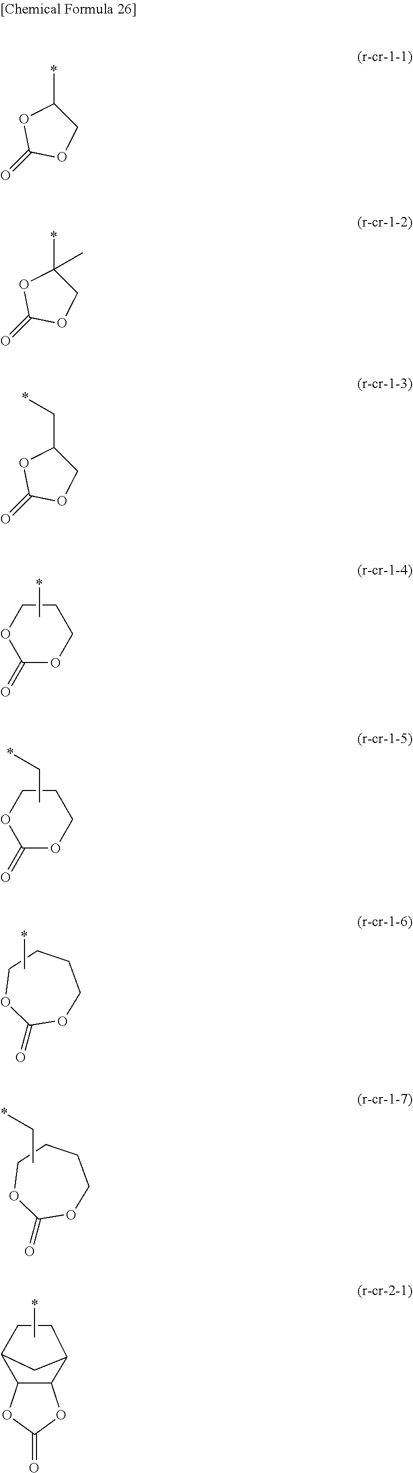

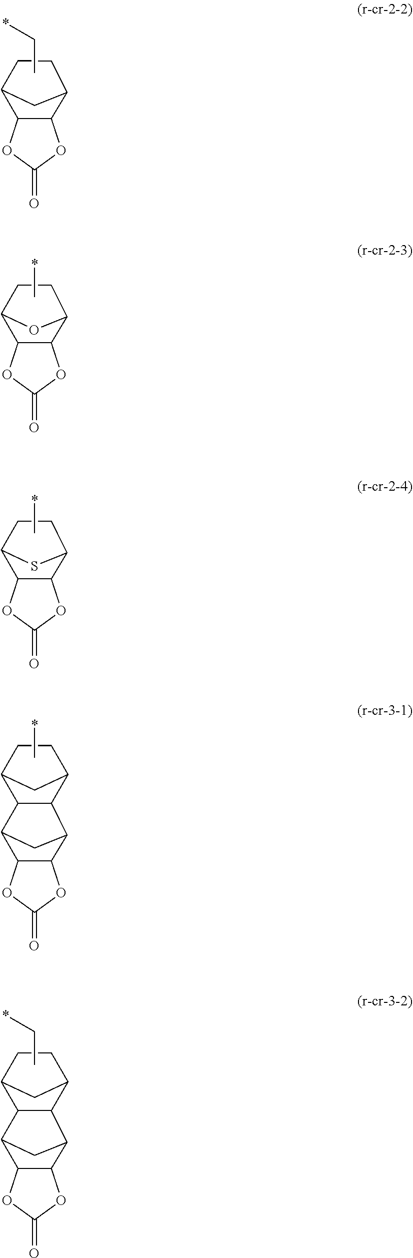

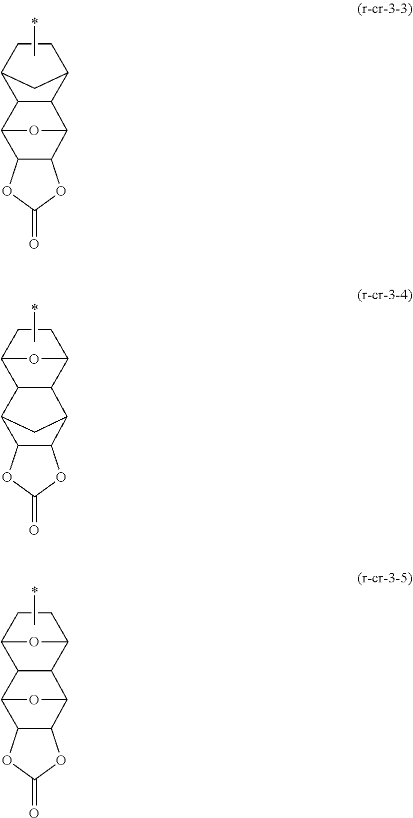

As the structural unit (a2), a structural unit derived from an acrylate ester which may have the hydrogen atom bonded to the carbon atom on the α-position substituted with a substituent is preferable.

Specific examples of preferable structural units for the structural unit (a2) include structural units represented by general formula (a2-1) shown below.

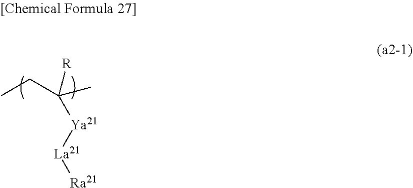

In the formula, R represents a hydrogen atom, an alkyl group of 1 to 5 carbon atoms or a halogenated alkyl group of 1 to 5 carbon atoms; Ya²¹ represents a single bond or a divalent linking group; La²¹ represents —O—, —COO—, —CON(R′)—, —OCO—, —CONHCO— or —CONHCS—; and R′ represents a hydrogen atom or a methyl group; provided that, when La²¹ represents —O—, Ya²¹ does not represent —CO—; and Ra²¹ represents a lactone-containing cyclic group, an —SO₂-containing cyclic group or a carbonate-containing cyclic group.

In the formula (a2-1), R is the same as defined above.

As R, a hydrogen atom, an alkyl group of 1 to 5 carbon atoms or a fluorinated alkyl group of 1 to 5 carbon atoms is preferable, and a hydrogen atom or a methyl group is particularly desirable in terms of industrial availability.

In formula (a2-1), the divalent linking group for Ya²¹ is the same as defined above for the divalent linking group represented by La⁰¹ and La⁰² in the aforementioned general formula (a0-1).

Ya²¹ preferably represents an ester bond [—C(═O)—O—], an ether bond (—O—), a linear or branched alkylene group, a combination of these, or a single bond.

In formula (a2-1), La²¹ represents —O—, —OCO—, —CON(R′)—, —OCO—, —CONHCO— or —CONHCS—.

R′ represents a hydrogen atom or a methyl group.

However, when La²¹ represents —O—, Ya²¹ does not represent —CO—.

In formula (a2-1), Ra²¹ represents a lactone-containing cyclic group or a carbonate-containing cyclic group.

Preferable examples of the lactone-containing cyclic group and the carbonate-containing cyclic group for Ra²¹ include groups represented by general formulae (a2-r-1) to (a2-r-7), and groups represented by general formulae (ax3-r-1) to (ax3-r-3).

Among as Ra²¹, a lactone-containing cyclic group is preferable, and a group represented by the aforementioned general formula (a2-r-1), (a2-r-2) or (a2-r-6) is more preferable. Specifically, a group represented by any of the aforementioned chemical formulae (r-1c-1-1) to (r-1c-1-7), (r-1c-2-1) to (r-1c-2-18), and (r-1c-6-1) is still more preferable, and a group represented by the aforementioned chemical formula (r-1c-1-1) or (r-1c-6-1) is still further preferable.

As the structural unit (a2) contained in the component (A1), 1 kind of structural unit may be used, or 2 or more kinds may be used.

When the component (A1) includes the structural unit (a2), the amount of the structural unit (a2) based on the combined total (100 mol %) of all structural units constituting the component (A1) is preferably 1 to 70 mol %, more preferably 3 to 60 mol %, and still more preferably 5 to 50 mol %.

When the amount of the structural unit (a2) is at least as large as the lower limit of the above preferable range, the effect of using the structural unit (a2) may be satisfactorily achieved. On the other hand, when the amount of the structural unit (a2) is no more than the upper limit of the above preferable range, a good balance may be achieved with the other structural units, and various lithography properties and pattern shape may be improved.

[Structural Unit (a3)]

The component (A1) may include, in addition to the structural unit (a0), a structural unit (a3) containing a polar group-containing aliphatic hydrocarbon group (provided that the structural units that fall under the definition of structural units (a0), (a1) and (a2) are excluded).

When the component (A1) includes the structural unit (a3), the hydrophilicity of the component (A1) is enhanced, thereby contributing to improvement in resolution.

Examples of the polar group include a hydroxyl group, cyano group, carboxyl group, or hydroxyalkyl group in which part of the hydrogen atoms of the alkyl group have been substituted with fluorine atoms, although a hydroxyl group is particularly desirable.

Examples of the aliphatic hydrocarbon group include linear or branched hydrocarbon groups (preferably alkylene groups) of 1 to 10 carbon atoms, and cyclic aliphatic hydrocarbon groups (cyclic groups). These cyclic groups may be selected appropriately from the multitude of groups that have been proposed for the resins of resist compositions designed for use with ArF excimer lasers. The cyclic group is preferably a polycyclic group, more preferably a polycyclic group of 7 to 30 carbon atoms.

Of the various possibilities, structural units derived from an acrylate ester that include an aliphatic polycyclic group that contains a hydroxyl group, cyano group, carboxyl group or a hydroxyalkyl group in which part of the hydrogen atoms of the alkyl group have been substituted with fluorine atoms are particularly desirable. Examples of the polycyclic group include groups in which two or more hydrogen atoms have been removed from a bicycloalkane, tricycloalkane, tetracycloalkane or the like. Specific examples include groups in which two or more hydrogen atoms have been removed from a polycycloalkane such as adamantane, norbornane, isobornane, tricyclodecane or tetracyclododecane. Of these polycyclic groups, groups in which two or more hydrogen atoms have been removed from adamantane, norbornane or tetracyclododecane are preferred industrially.

As the structural unit (a3), there is no particular limitation as long as it is a structural unit containing a polar group-containing aliphatic hydrocarbon group, and an arbitrary structural unit may be used.

The structural unit (a3) is preferably a structural unit derived from an acrylate ester which may have the hydrogen atom bonded to the carbon atom on the α-position substituted with a substituent and contains a polar group-containing aliphatic hydrocarbon group.

When the aliphatic hydrocarbon group within the polar group-containing aliphatic hydrocarbon group is a linear or branched hydrocarbon group of 1 to 10 carbon atoms, the structural unit (a3) is preferably a structural unit derived from a hydroxyethyl ester of acrylic acid. On the other hand, when the hydrocarbon group is a polycyclic group, structural units represented by formulas (a3-1), (a3-2) and (a3-3) shown below are preferable.

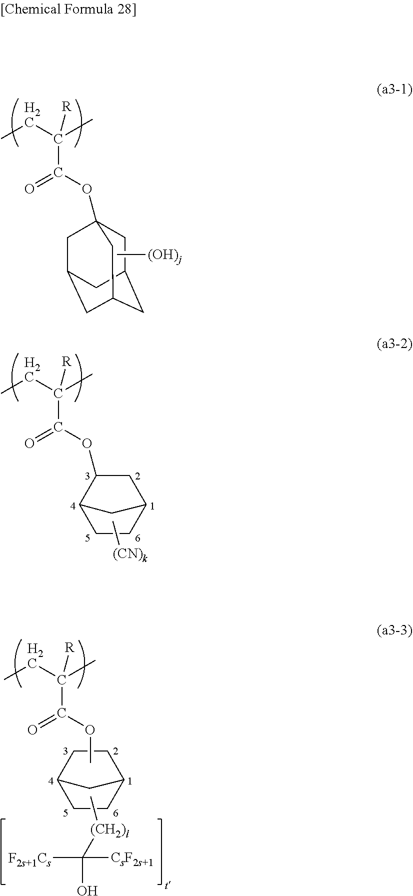

In the formulas, R is the same as defined above; j is an integer of 1 to 3; k is an integer of 1 to 3; t′ is an integer of 1 to 3; l is an integer of 1 to 5; and s is an integer of 1 to 3.

In formula (a3-1), j is preferably 1 or 2, and more preferably 1. When j is 2, it is preferable that the hydroxyl groups be bonded to the 3rd and 5th positions of the adamantyl group. When j is 1, it is preferable that the hydroxyl group be bonded to the 3rd position of the adamantyl group.

j is preferably 1, and it is particularly desirable that the hydroxyl group be bonded to the 3rd position of the adamantyl group.

In formula (a3-2), k is preferably 1. The cyano group is preferably bonded to the 5th or 6th position of the norbornyl group.

In formula (a3-3), t′ is preferably 1. l is preferably 1. s is preferably 1. Further, it is preferable that a 2-norbornyl group or 3-norbornyl group be bonded to the terminal of the carboxy group of the acrylic acid. The fluorinated alkyl alcohol is preferably bonded to the 5th or 6th position of the norbornyl group.

As the structural unit (a3) contained in the component (A1), 1 kind of structural unit may be used, or 2 or more kinds of structural units may be used.

When the component (A1) contains the structural unit (a3), the amount of the structural unit (a3) based on the combined total (100 mol %) of all structural units constituting the component (A1) is preferably 1 to 50 mol %, more preferably 1 to 40 mol %, still more preferably 3 to 30 mol %, and most preferably 5 to 20 mol %.

When the amount of the structural unit (a3) is at least as large as the lower limit of the above-mentioned preferable range, the resolution is improved in the formation of a resist pattern. On the other hand, when the amount of the structural unit (a3) is no more than the upper limit of the above-mentioned preferable range, a good balance may be reliably achieved with the other structural units.

[Structural Unit (a4)]

The component (A1) may be further include, in addition to the structural unit (a0), a structural unit (a4) containing an acid non-dissociable, aliphatic cyclic group.

When the component (A1) includes the structural unit (a4), dry etching resistance of the resist pattern to be formed is improved. Further, the hydrophobicity of the component (A) is further improved. Increase in the hydrophobicity contributes to improvement in terms of resolution, shape of the resist pattern and the like, particularly in a solvent developing process.

An “acid non-dissociable, aliphatic cyclic group” in the structural unit (a4) refers to a cyclic group which is not dissociated by the action of the acid (e.g., acid generated from a structural unit which generates acid upon exposure or acid generated from the component (B)) upon exposure, and remains in the structural unit.

As the structural unit (a4), a structural unit which contains a non-acid-dissociable aliphatic cyclic group, and is also derived from an acrylate ester is preferable. As the cyclic group, any of the multitude of conventional polycyclic groups used within the resin component of resist compositions for ArF excimer lasers or KrF excimer lasers (and particularly for ArF excimer lasers) may be used.

As the aliphatic polycyclic group, at least one polycyclic group selected from amongst a tricyclodecyl group, adamantyl group, tetracyclododecyl group, isobornyl group, and norbornyl group is particularly desirable in consideration of industrial availability and the like. These polycyclic groups may be substituted with a linear or branched alkyl group of 1 to 5 carbon atoms.

Specific examples of the structural unit (a4) include structural units represented by general formulae (a4-1) to (a4-7) shown below.

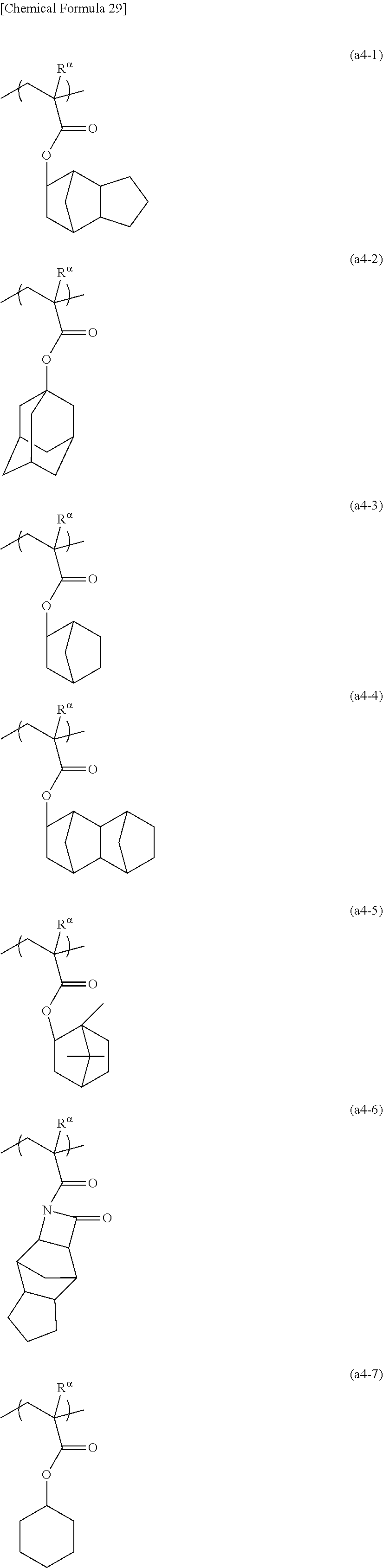

In the formulae, Ra is the same as defined above.

As the structural unit (a4) contained in the component (A1), 1 type of structural unit may be used, or 2 or more types may be used.

When the component (A1) contains the structural unit (a4), the amount of the structural unit (a4) based on the combined total (100 mol %) of all structural units constituting the component (A1) is preferably 1 to 40 mol %, and more preferably 5 to 20 mol %.

When the amount of the structural unit (a4) is at least as large as the lower limit of the above-mentioned preferable range, the effect of using the structural unit (a4) may be satisfactorily achieved. On the other hand, when the amount of the structural unit (a4) is no more than the upper limit of the above-mentioned preferable range, a good balance may be achieved with the other structural units.

Structural Unit Derived from Styrene or a Derivative Thereof (Structural Unit (St))

The term “styrene” is a concept including styrene and compounds in which the hydrogen atom at the α-position of styrene is substituted with a substituent such as an alkyl group or a halogenated alkyl group. Examples of the alkyl group as the substituent include an alkyl group having 1 to 5 carbon atoms. Examples of the halogenated alkyl group as the substituent include a halogenated alkyl group having 1 to 5 carbon atoms.

Examples of the “styrene derivative” include styrene which has a substituent other than a hydroxy group bonded to the benzene ring and may have the hydrogen atom on the α-position substituted with a substituent.

Here, the α-position (carbon atom on the α-position) refers to the carbon atom having the benzene ring bonded thereto, unless specified otherwise.

A “structural unit derived from styrene” or “structural unit derived from a styrene derivative” refers to a structural unit that is formed by the cleavage of the ethylenic double bond of styrene or a styrene derivative.

As the structural unit (st) contained in the component (A1), 1 kind of structural unit may be used, or 2 or more kinds of structural units may be used.

When the component (A1) includes the structural unit (st), the amount of the structural unit (st) based on the combined total of all structural units constituting the component (A1) (100 mol %) is preferably 1 to 30 mol %, and more preferably 3 to 20 mol %.

In the resist composition according to the present embodiment, the component (A1) is preferably a copolymer (component (A1-1)) having a structural unit (a0) and any other desired structural unit.

Specific examples of the component (A1-1) include a polymeric compound consisting of a repeating structure of the structural unit (a0) and the structural unit (a1); a polymeric compound consisting of a repeating structure of the structural unit (a0), the structural unit (a1) and the structural unit (a2); and a polymeric compound consisting of a repeating structure of the structural unit (a0), the structural unit (a1), the structural unit (a2) and the structural unit (a3).

The weight average molecular weight (Mw) (the polystyrene equivalent value determined by gel permeation chromatography (GPC)) of the component (A1) is not particularly limited, but is preferably 1,000 to 50,000, more preferably 2,000 to 30,000, and still more preferably 3,000 to 20,000.

When the Mw of the component (A1) is no more than the upper limit of the above-mentioned preferable range, the resist composition exhibits a satisfactory solubility in a resist solvent. On the other hand, when the Mw of the component (A1) is at least as large as the lower limit of the above-mentioned preferable range, dry etching resistance and the cross-sectional shape of the resist pattern becomes satisfactory.

The dispersity (Mw/Mn) of the component (A1) is not particularly limited, but is preferably 1.0 to 4.0, more preferably 1.0 to 3.0, and most preferably 1.5 to 2.5. Here, Mn is the number average molecular weight.

As the component (A1), one kind may be used alone, or two or more kinds may be used in combination.

In the component (A), the amount of the component (A1) based on the total weight of the component (A) is preferably 25% by weight or more, more preferably 50% by weight or more, still more preferably 75% by weight or more, and may be even 100% by weight. When the amount of the component (A1) is 25% by weight or more, a resist pattern with improved lithography properties may be reliably formed, such as improved sensitivity and improved roughness.

Production Method of Component (A1):

The component (A1) may be produced, for example, by dissolving the monomers corresponding with each of the structural units in a polymerization solvent, followed by addition of a radical polymerization initiator such as azobisisobutyronitrile (AIBN) or dimethyl-2,2′-azobisisoutyrate (e.g., V-601). Furthermore, in the component (F), by using a chain transfer agent such as HS—CH₂—CH₂—CH₂—C(CF₃)₂—OH, a —C(CF₃)₂—OH group may be introduced at the terminals of the component (F). Such a copolymer having introduced a hydroxyalkyl group in which some of the hydrogen atoms of the alkyl group are substituted with fluorine atoms is effective in reducing developing defects and LER (line edge roughness: unevenness of the side walls of a line pattern).

In the resist composition of the present embodiment, as the component (A), one kind of compound may be used, or two or more kinds of compounds may be used in combination.

In the resist composition of the present embodiment, the amount of the component (A) may be appropriately adjusted depending on the thickness of the resist film to be formed, and the like.

<<Component (A2)>>

In the resist composition of the present embodiment, as the component (A), “a base component which exhibits changed solubility in a developing solution under action of acid” other than the component (A1) (hereafter, referred to as “component (A2)”) may be used in combination.

As the component (A2), there is no particular limitation, and any of the multitude of conventional base resins used within chemically amplified resist compositions may be arbitrarily selected for use.

As the component (A2), one kind of a polymer or a low molecular weight compound may be used, or a combination of two or more kinds may be used.

In the resist composition of the present embodiment, the amount of the component (A) may be appropriately adjusted depending on the thickness of the resist film to be formed, and the like.

<Other Components>

The resist composition of the present embodiment may contain, in addition to the aforementioned component (A), any other components other than the component (A). Examples of the other components include the component (B), the component (D), the component (E), the component (F) and the component (S) described below.

<<Acid-Generator Component (B)>>

The resist composition of the present embodiment may include, in addition to the components (A), an acid-generator component (hereafter, sometimes referred to as “component (B)”).

As the component (B), there is no particular limitation, and any of the known acid generators used in conventional chemically amplified resist compositions may be used.

Examples of these acid generators are numerous, and include onium salt acid generators such as iodonium salts and sulfonium salts; oxime sulfonate acid generators; diazomethane acid generators such as bisalkyl or bisaryl sulfonyl diazomethanes and poly(bis-sulfonyl)diazomethanes; nitrobenzylsulfonate acid generators; iminosulfonate acid generators; and disulfone acid generators. Among these, it is preferable to use an onium salt acid generator.

As the onium salt acid generator, a compound represented by general formula (b-1) below (hereafter, sometimes referred to as “component (b-1)”), a compound represented by general formula (b-2) below (hereafter, sometimes referred to as “component (b-2)”) or a compound represented by general formula (b-3) below (hereafter, sometimes referred to as “component (b-3)”) may be used.

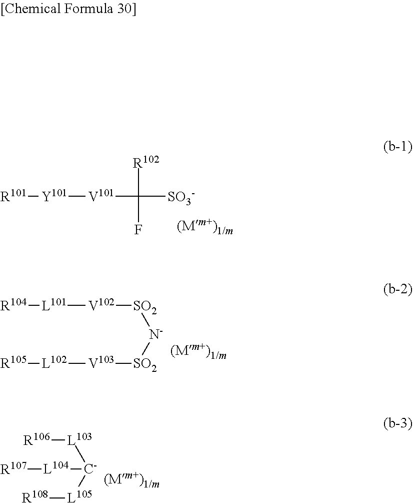

In the formulae, R¹⁰¹ and R¹⁰⁴ to R¹⁰⁸ each independently represents a cyclic group which may have a substituent, a chain-like alkyl group which may have a substituent or a chain-like alkenyl group which may have a substituent, provided that R¹⁰⁴ and R¹⁰⁵ may be mutually bonded to form a ring;

R¹⁰² represents a fluorine atom or a fluorinated alkyl group of 1 to 5 carbon atoms; Y¹⁰¹ represents a single bond or a divalent linking group containing an oxygen atom; V¹⁰¹ to V¹⁰³ each independently represents a single bond, an alkylene group or a fluorinated alkylene group; L¹⁰¹ and L¹⁰² each independently represents a single bond or an oxygen atom; L¹⁰³ to L¹⁰⁵ each independently represents a single bond, —CO— or —SO₂—; m represents an integer of 1 or more; and M′^{m+} represents an m-valent onium cation.

{Anion Moiety}

Anion Moiety of Component (b-1)

In the formula (b-1), R¹⁰¹ represents a cyclic group which may have a substituent, a chain-like alkyl group which may have a substituent or a chain-like alkenyl group which may have a substituent.

Cyclic Group which May have a Substituent:

The cyclic group is preferably a cyclic hydrocarbon group, and the cyclic hydrocarbon group may be either an aromatic hydrocarbon group or an aliphatic hydrocarbon group. An “aliphatic hydrocarbon group” refers to a hydrocarbon group that has no aromaticity. The aliphatic hydrocarbon group may be either saturated or unsaturated, but in general, the aliphatic hydrocarbon group is preferably saturated.

The aromatic hydrocarbon group for R¹⁰¹ is a hydrocarbon group having an aromatic ring. The aromatic hydrocarbon group preferably has 3 to 30 carbon atoms, more preferably 5 to 30 carbon atoms, still more preferably 5 to 20 carbon atoms, still more preferably 6 to 15 carbon atoms, and most preferably 6 to 10 carbon atoms. Here, the number of carbon atoms within a substituent(s) is not included in the number of carbon atoms of the aromatic hydrocarbon group.

Examples of the aromatic ring contained in the aromatic hydrocarbon group represented by R¹⁰¹ include benzene, fluorene, naphthalene, anthracene, phenanthrene and biphenyl; and aromatic hetero rings in which part of the carbon atoms constituting the aforementioned aromatic rings has been substituted with a hetero atom. Examples of the hetero atom within the aromatic hetero rings include an oxygen atom, a sulfur atom and a nitrogen atom.

Specific examples of the aromatic hydrocarbon group represented by R¹⁰¹ include a group in which one hydrogen atom has been removed from the aforementioned aromatic ring (i.e., an aryl group, such as a phenyl group or a naphthyl group), and a group in which one hydrogen of the aforementioned aromatic ring has been substituted with an alkylene group (e.g., an arylalkyl group such as a benzyl group, a phenethyl group, a 1-naphthylmethyl group, a 2-naphthylmethyl group, a 1-naphthylethyl group or a 2-naphthylethyl group). The alkylene group (alkyl chain within the arylalkyl group) preferably has 1 to 4 carbon atom, more preferably 1 or 2, and most preferably 1.

Examples of the cyclic aliphatic hydrocarbon group for R¹⁰¹ include aliphatic hydrocarbon groups containing a ring in the structure thereof.

As examples of the hydrocarbon group containing a ring in the structure thereof, an alicyclic hydrocarbon group (a group in which one hydrogen atom has been removed from an aliphatic hydrocarbon ring), a group in which the alicyclic hydrocarbon group is bonded to the terminal of the aforementioned chain-like aliphatic hydrocarbon group, and a group in which the alicyclic group is interposed within the aforementioned linear or branched aliphatic hydrocarbon group, may be given.

The alicyclic hydrocarbon group preferably has 3 to 20 carbon atoms, and more preferably 3 to 12 carbon atoms.

The alicyclic hydrocarbon group may be either a polycyclic group or a monocyclic group. As the monocyclic alicyclic hydrocarbon group, a group in which one or more hydrogen atoms have been removed from a monocycloalkane is preferable. The monocycloalkane preferably has 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the polycyclic group preferably has 7 to 30 carbon atoms. Among polycycloalkanes, a polycycloalkane having a bridged ring polycyclic skeleton, such as adamantane, norbornane, isobornane, tricyclodecane or tetracyclodpdecane, and a polycycloalkane having a condensed ring polycyclic skeleton, such as a cyclic group having a steroid skeleton are preferable.

Among these examples, as the cyclic aliphatic hydrocarbon group for R¹⁰¹, a group in which one or more hydrogen atoms have been removed from a monocycloalkane or a polycycloalkane is preferable, a group in which one or more hydrogen atoms have been removed from a polycycloalkane is more preferable, an adamantyl group or a norbornyl group is still more preferable, and an adamantyl group is most preferable.

The linear or branched aliphatic hydrocarbon group which may be bonded to the alicyclic hydrocarbon group preferably has 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄-] and a pentamethylene group [—(CH₂)₅—].

As the branched aliphatic hydrocarbon group, branched alkylene groups are preferred, and specific examples include various alkylalkylene groups, including alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂—, and —CH₂CH(CH₃)CH₂CH₂—. As the alkyl group within the alkylalkylene group, a linear alkyl group of 1 to 5 carbon atoms is preferable.

The cyclic hydrocarbon group for R¹⁰¹ may contain a hetero atom such as a heterocycle. Specific examples include lactone-containing cyclic groups represented by the aforementioned general formulae (a2-r-1) to (a2-r-7), the —SO₂-containing cyclic group represented by the aforementioned formulae (a5-r-1) to (a5-r-4), and other heterocyclic groups represented by chemical formulae (r-hr-1) to (r-hr-16) shown below.

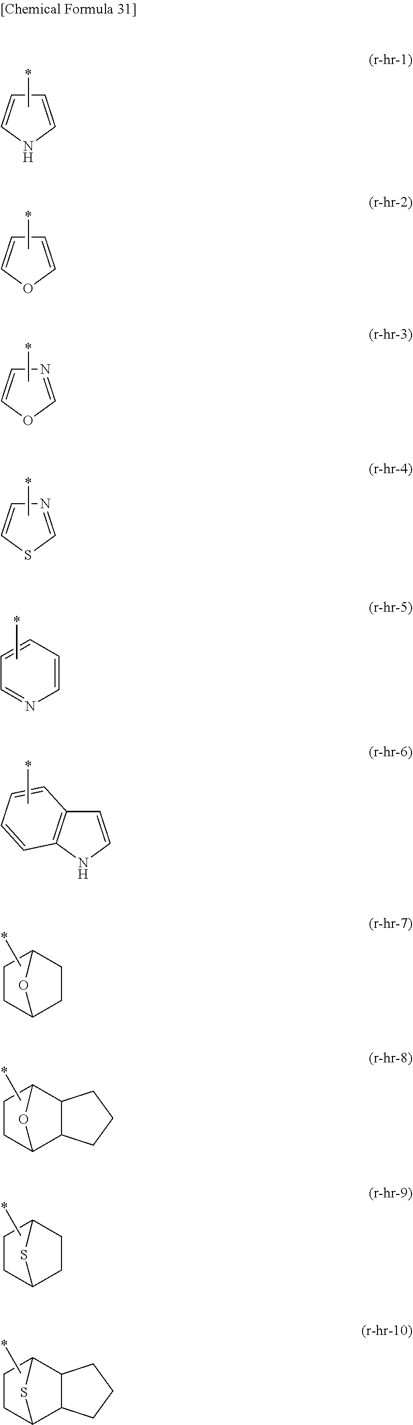

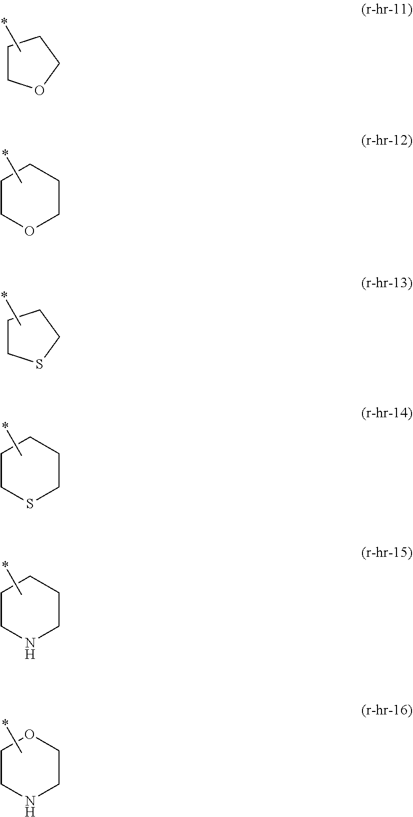

As the substituent for the cyclic group for R¹⁰¹, an alkyl group, an alkoxy group, a halogen atom, a halogenated alkyl group, a hydroxyl group, a carbonyl group, a nitro group or the like may be used.

The alkyl group as the substituent is preferably an alkyl group of 1 to 5 carbon atoms, and a methyl group, an ethyl group, a propyl group, an n-butyl group or a tert-butyl group is particularly desirable.

The alkoxy group as the substituent is preferably an alkoxy group having 1 to 5 carbon atoms, more preferably a methoxy group, ethoxy group, n-propoxy group, iso-propoxy group, n-butoxy group or tert-butoxy group, and most preferably a methoxy group or an ethoxy group.

Examples of the halogen atom for the substituent include a fluorine atom, a chlorine atom, a bromine atom and an iodine atom, and a fluorine atom is preferable.

Example of the aforementioned halogenated alkyl group includes a group in which a part or all of the hydrogen atoms within an alkyl group of 1 to 5 carbon atoms (e.g., a methyl group, an ethyl group, a propyl group, an n-butyl group or a tert-butyl group) have been substituted with the aforementioned halogen atoms.

The carbonyl group as the substituent is a group that substitutes a methylene group (—CH₂—) constituting the cyclic hydrocarbon group.

Chain alkyl group which may have a substituent:

The chain-like alkyl group for R¹⁰¹ may be linear or branched.

The linear alkyl group preferably has 1 to 20 carbon atoms, more preferably 1 to 15 carbon atoms, and still more preferably 1 to 10 carbon atoms. Specific examples include a methyl group, an ethyl group, a propyl group, a butyl group, a pentyl group, a hexyl group, a heptyl group, an octyl group, a nonyl group, a decyl group, an undecyl group, a dodecyl group, a tridecyl group, an isotridecyl group, a tetradecyl group, a pentadecyl group, a hexadecyl group, an isohexadecyl group, a heptadecyl group, an octadecyl group, a nonadecyl group, an icosyl group, a henicosyl group and a docosyl group.

The branched alkyl group preferably has 3 to 20 carbon atoms, more preferably 3 to 15 carbon atoms, and still more preferably 3 to 10 carbon atoms. Specific examples include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group and a 4-methylpentyl group.

Chain alkenyl group which may have a substituent:

The chain-like alkenyl group for R¹⁰¹ may be linear or branched, and preferably has 2 to 10 carbon atoms, more preferably 2 to 5 carbon atoms, still more preferably 2 to 4 carbon atoms, and most preferably 3 carbon atoms. Examples of linear alkenyl groups include a vinyl group, a propenyl group (an allyl group) and a butynyl group. Examples of branched alkenyl groups include a 1-methylvinyl group, a 2-methylvinyl group, a 1-methylpropenyl group and a 2-methylpropenyl group.

Among these examples, as the chain-like alkenyl group, a linear alkenyl group is preferable, a vinyl group or a propenyl group is more preferable, and a vinyl group is most preferable.

As the substituent for the chain-like alkyl group or alkenyl group for R¹⁰¹, an alkoxy group, a halogen atom, a halogenated alkyl group, a hydroxyl group, a carbonyl group, a nitro group, an amino group, a cyclic group for R¹⁰¹ or the like may be used.

Among these examples, as R¹⁰¹, a cyclic group which may have a substituent is preferable, and a cyclic hydrocarbon group which may have a substituent is more preferable. Specifically, for example, a phenyl group, a naphthyl group, a group in which one or more hydrogen atoms have been removed from a polycycloalkane, a lactone-containing cyclic group represented by any one of the aforementioned formula (a2-r-1) to (a2-r-7), and an —SO₂-containing cyclic group represented by any one of the aforementioned formula (a5-r-1) to (a5-r-4) are preferable.

In formula (b-1), Y¹⁰¹ represents a single bond or a divalent linking group containing an oxygen atom.

In the case where Y¹⁰¹ is a divalent linking group containing an oxygen atom, Y¹⁰¹ may contain an atom other than an oxygen atom. Examples of atoms other than an oxygen atom include a carbon atom, a hydrogen atom, a sulfur atom and a nitrogen atom.

Examples of divalent linking groups containing an oxygen atom include non-hydrocarbon, oxygen atom-containing linking groups such as an oxygen atom (an ether bond; —O—), an ester bond (—C(═O)—O—), an oxycarbonyl group (—O—C(═O)—), an amido bond (—C(═O)—NH—), a carbonyl group (—C(═O)—) and a carbonate bond (—O—C(═O)—O—); and combinations of the aforementioned non-hydrocarbon, hetero atom-containing linking groups with an alkylene group. Furthermore, the combinations may have a sulfonyl group (—SO₂—) bonded thereto. Examples of divalent linking groups containing an oxygen atom include linking groups represented by general formulae (y-a1-1) to (y-a1-7) shown below.

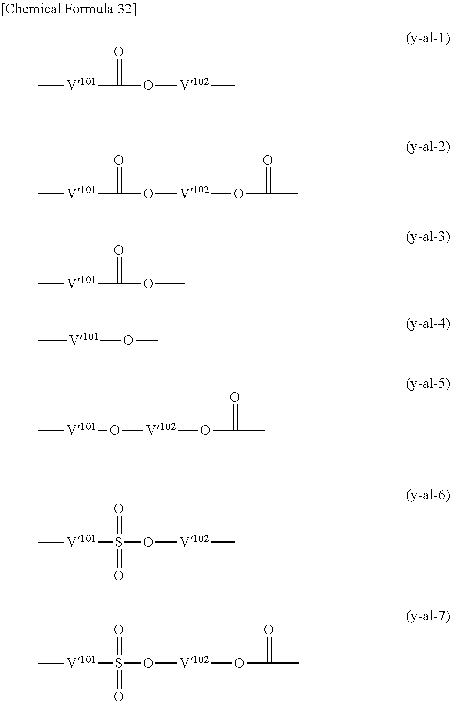

In the formulae, V′¹⁰¹ represents a single bond or an alkylene group of 1 to 5 carbon atoms; V′¹⁰² represents a divalent saturated hydrocarbon group of 1 to 30 carbon atoms.

The divalent saturated hydrocarbon group for V′¹⁰² is preferably an alkylene group of 1 to 30 carbon atoms, more preferably an alkylene group of 1 to 10 carbon atoms, and still more preferably an alkylene group of 1 to 5 carbon atoms.

The alkylene group for V′¹⁰¹ and V′¹⁰² may be a linear alkylene group or a branched alkylene group, and a linear alkylene group is preferable.

Specific examples of the alkylene group for V′¹⁰¹ and V′¹⁰² include a methylene group [—CH₂—]; an alkylmethylene group, such as —CH(CH₃)—, —CH(CH₂CH₃)—, —C(CH₃)₂—, —C(CH₃)(CH₂CH₃)—, —C(CH₃)(CH₂CH₂CH₃)— and —C(CH₂CH₃)₂—; an ethylene group [—CH₂CH₂—]; an alkylethylene group, such as —CH(CH₃)CH₂—, —CH(CH₃)CH(CH₃)—, —C(CH₃)₂CH₂— and —CH(CH₂CH₃)CH₂—; a trimethylene group (n-propylene group) [—CH₂CH₂CH₂—]; an alkyltrimethylene group, such as —CH(CH₃)CH₂CH₂— and —CH₂CH(CH₃)CH₂—; a tetramethylene group [—CH₂CH₂CH₂CH₂—]; an alkyltetramethylene group, such as —CH(CH₃)CH₂CH₂CH₂—, —CH₂CH(CH₃)CH₂CH₂—; and a pentamethylene group [—CH₂CH₂CH₂CH₂CH₂-].

Further, part of methylene group within the alkylene group for V′¹⁰¹ and V′¹⁰² may be substituted with a divalent aliphatic cyclic group of 5 to 10 carbon atoms. The aliphatic cyclic group is preferably a divalent group in which one hydrogen atom has been removed from the cyclic aliphatic hydrocarbon group (monocyclic alicyclic hydrocarbon group or polycyclic alicyclic hydrocarbon group) for R¹⁰¹ in the aforementioned formula (b-1), and a cyclohexylene group, 1,5-adamantylene group or 2,6-adamantylene group is preferable.

Y¹⁰¹ is preferably a divalent linking group containing an ether bond or a divalent linking group containing an ester bond, and groups represented by the aforementioned formulas (y-a1-1) to (y-a1-5) are preferable.

In formula (b-1), V¹⁰¹ represents a single bond, an alkylene group or a fluorinated alkylene group. The alkylene group and the fluorinated alkylene group for V¹⁰¹ preferably has 1 to 4 carbon atoms. Examples of the fluorinated alkylene group for V¹⁰¹ include a group in which part or all of the hydrogen atoms within the alkylene group for V¹⁰¹ have been substituted with fluorine. Among these examples, as V¹⁰¹, a single bond or a fluorinated alkylene group of 1 to 4 carbon atoms is preferable.

In formula (b-1), R¹⁰² represents a fluorine atom or a fluorinated alkyl group of 1 to 5 carbon atoms. R¹⁰² is preferably a fluorine atom or a perfluoroalkyl group of 1 to 5 carbon atoms, and more preferably a fluorine atom.

As a specific example of the anion moiety for the component (b-1), in the case where Y¹⁰¹ a single bond, a fluorinated alkylsulfonate anion such as a trifluoromethanesulfonate anion or a perfluorobutanesulfonate anion may be mentioned; and in the case where Y¹⁰¹ represents a divalent linking group containing an oxygen atom, anions represented by formulae (an-1) to (an-3) shown below may be mentioned.

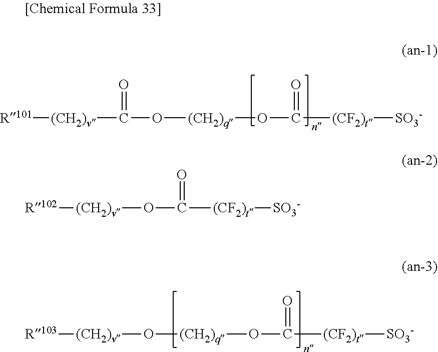

In the formulae, R″¹⁰¹ represents an aliphatic cyclic group which may have a substituent, a group represented by any one of the aforementioned formulas (r-hr-1) to (r-hr-6) or a chain-like alkyl group which may have a substituent; R″¹⁰² represents an aliphatic cyclic group which may have a substituent, a lactone-containing cyclic group represented by any one of the aforementioned formulae (a2-r-1) to (a2-r-7) or an —SO₂-containing cyclic group represented by any one of the aforementioned formulae (a5-r-1) to (a5-r-4); R″¹⁰³ represents an aromatic cyclic group which may have a substituent, an aliphatic cyclic group which may have a substituent or a chain-like alkenyl group which may have a substituent; v″ represents an integer of 0 to 3; q″ represents an integer of 1 to 20; t″ represents an integer of 1 to 3; and n″ represents 0 or 1.

As the aliphatic cyclic group for R″¹⁰¹, R″¹⁰² and R″¹⁰³ which may have a substituent, the same groups as the cyclic aliphatic hydrocarbon group for R¹⁰¹ described above are preferable. As the substituent, the same groups as those described above for substituting the cyclic aliphatic hydrocarbon group for R¹⁰¹ may be mentioned.

As the aromatic cyclic group for R″¹⁰³ which may have a substituent, the same groups as the aromatic hydrocarbon group for the cyclic hydrocarbon group represented by R¹⁰¹ described above are preferable. The substituent is the same as defined for the substituent for the aromatic hydrocarbon group represented by R¹⁰¹.

As the chain-like alkyl group for R″¹⁰¹ which may have a substituent, the same groups as those described above for R¹⁰¹ are preferable. As the chain-like alkenyl group for R″¹⁰³ which may have a substituent, the same groups as those described above for R¹⁰¹ are preferable.

Anion Moiety of Component (b-2)

In formula (b-2), R¹⁰⁴ and R¹⁰⁵ each independently represents a cyclic group which may have a substituent, a chain-like alkyl group which may have a substituent or a chain-like alkenyl group which may have a substituent, and is the same as defined for R¹⁰¹ in formula (b-1). R¹⁰⁴ and R¹⁰⁵ may be mutually bonded to form a ring.

As R¹⁰⁴ and R¹⁰⁵, a chain-like alkyl group which may have a substituent is preferable, and a linear or branched alkyl group or a linear or branched fluorinated alkyl group is more preferable.

The chain-like alkyl group preferably has 1 to 10 carbon atoms, more preferably 1 to 7 carbon atoms, and still more preferably 1 to 3 carbon atoms. The smaller the number of carbon atoms of the chain-like alkyl group for R¹⁰⁴ and R¹⁰⁵, the more the solubility in a resist solvent is improved. Further, in the chain-like alkyl group for R¹⁰⁴ and R¹⁰⁵, it is preferable that the number of hydrogen atoms substituted with fluorine atoms is as large as possible because the acid strength increases and the transparency to high energy radiation of 200 nm or less or electron beam is improved.

The fluorination ratio of the chain-like alkyl group is preferably from 70 to 100%, more preferably from 90 to 100%, and it is particularly desirable that the chain-like alkyl group be a perfluoroalkyl group in which all hydrogen atoms are substituted with fluorine atoms.

In formula (b-2), V¹⁰² and V¹⁰³ each independently represents a single bond, an alkylene group or a fluorinated alkylene group, and is the same as defined for V¹⁰¹ in formula (b-1).

In formula (b-2), L¹⁰¹ and L¹⁰² each independently represents a single bond or an oxygen atom.

Anion Moiety of Component (b-3)

In formula (b-3), R¹⁰⁶ to R¹⁰⁸ each independently represents a cyclic group which may have a substituent, a chain-like alkyl group which may have a substituent or a chain-like alkenyl group which may have a substituent, and is the same as defined for R¹⁰¹ in formula (b-1).

L¹⁰³ to L¹⁰⁵ each independently represents a single bond, —CO— or —SO₂—.

{Cation Moiety}

In formulae (b-1), (b-2) and (b-3), m represents an integer of 1 or more, M′^{m+} represents an onium cation having a valency of m, preferably a sulfonium cation or an iodonium cation, and most preferably an organic cation represented by any one of the following formulae (ca-1) to (ca-5).

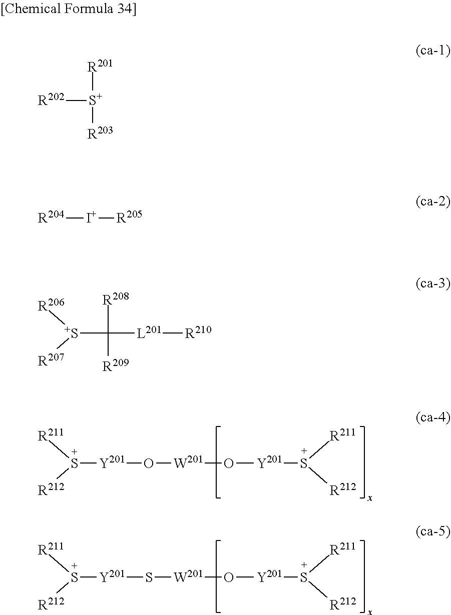

In the formulae, R²⁰¹ to R²⁰⁷, R²¹¹ and R²¹² each independently represents an aryl group, an alkyl group or an alkenyl group, provided that two of R²⁰¹ to R²⁰³, R²⁰⁶ and R²⁰⁷, or R²¹¹ and R²¹² may be mutually bonded to form a ring with the sulfur atom; R²⁰⁸ and R²⁰⁹ each independently represents a hydrogen atom or an alkyl group of 1 to 5 carbon atoms; R²¹⁰ represents an aryl group which may have a substituent, an alkyl group which may have a substituent, an alkenyl group which may have a substituent, or an —SO₂— containing cyclic group which may have a substituent; L²⁰¹ represents —C(═O)— or —C(═O)—O—; Y²⁰¹ each independently represents an arylene group, an alkylene group or an alkenylene group; x represents 1 or 2; and W²⁰¹ represents a linking group having a valency of (x+1).

As the aryl group for R²⁰¹ to R²⁰⁷, R²¹¹ and R²¹², an unsubstituted aryl group of 6 to 20 carbon atoms may be mentioned, and a phenyl group or a naphthyl group is preferable.

The alkyl group for R²⁰¹ to R²⁰⁷, R²¹¹ and R²¹² is preferably a chain-like or cyclic alkyl group having 1 to 30 carbon atoms.

The alkenyl group for R²⁰¹ to R²⁰⁷, R²¹¹ and R²¹² preferably has 2 to 10 carbon atoms.

Specific examples of the substituent which R²⁰¹ to R²⁰⁷ and R²¹⁰ to R²¹² may have include an alkyl group, a halogen atom, a halogenated alkyl group, a carbonyl group, a cyano group, an amino group, an aryl group, and groups represented by formulae (ca-r-1) to (ca-r-7) shown below.

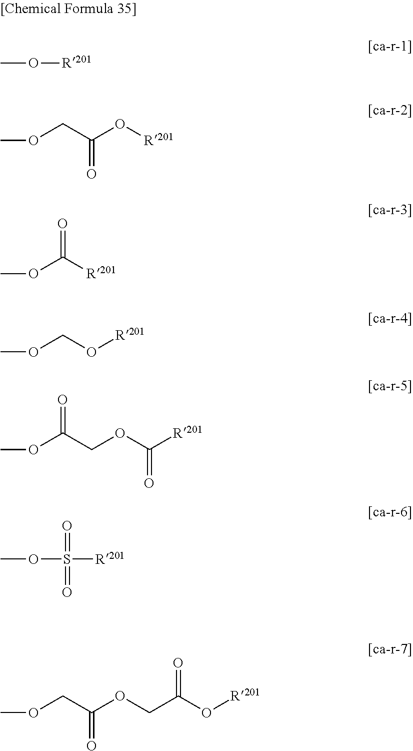

In the formulae, each R′²⁰¹ independently represents a hydrogen atom, a cyclic group which may have a substituent, a chain-like alkyl group which may have a substituent, or a chain-like alkenyl group which may have a substituent.

As the cyclic group which may have a substituent, the chain-like alkyl group which may have a substituent and the chain-like alkenyl group which may have a substituent for R′²⁰¹, the same groups as those described above for R¹⁰¹ may be mentioned. As the cyclic group which may have a substituent and chain-like alkyl group which may have a substituent, the same groups as those described above for the acid dissociable group represented by the aforementioned formula (a1-r-2) may be also mentioned.

When R²⁰¹ to R²⁰³, R²⁰⁶, R²⁰⁷, R²¹¹ and R²¹² are mutually bonded to form a ring with the sulfur atom, these groups may be mutually bonded via a hetero atom such as a sulfur atom, an oxygen atom or a nitrogen atom, or a functional group such as a carbonyl group, —SO—, —SO₂—, —SO₃—, —COO—, —CONH— or —N(RN)— (wherein RN represents an alkyl group of 1 to 5 carbon atoms). The ring containing the sulfur atom in the skeleton thereof is preferably a 3 to 10-membered ring, and most preferably a 5 to 7-membered ring. Specific examples of the ring formed include a thiophene ring, a thiazole ring, a benzothiophene ring, a thianthrene ring, a benzothiophene ring, a dibenzothiophene ring, a 9H-thioxanthene ring, a thioxanthone ring, a phenoxathiin ring, a tetrahydrothiophenium ring, and a tetrahydrothiopyranium ring.

R²⁰⁸ and R²⁰⁹ each independently represents a hydrogen atom or an alkyl group of 1 to 5 carbon atoms, preferably a hydrogen atom or an alkyl group of 1 to 3 carbon atoms, and when R²⁰⁸ and R²⁰⁹ each represents an alkyl group, R²⁰⁸ and R²⁰⁹ may be mutually bonded to form a ring.

R²¹⁰ represents an aryl group which may have a substituent, an alkyl group which may have a substituent, an alkenyl group which may have a substituent, or a —SO₂-containing cyclic group which may have a substituent.

Examples of the aryl group for R²¹⁰ include an unsubstituted aryl group of 6 to 20 carbon atoms, and a phenyl group or a naphthyl group is preferable.

As the alkyl group for R²¹⁰, a chain-like or cyclic alkyl group having 1 to 30 carbon atoms is preferable.

The alkenyl group for R²¹⁰ preferably has 2 to 10 carbon atoms.

The —SO₂-containing cyclic group for R²¹⁰ which may have a substituent is the same as defined for the —SO₂-containing cyclic group represented by any of the aforementioned general formulae (a5-r-1) to (a5-r-4). Among these examples, the “—SO₂-containing polycyclic group” is preferable, and a group represented by general formula (a5-r-1) is more preferable.

In formulae (ca-4) and (ca-5), each Y²⁰¹ independently represents an arylene group, an alkylene group or an alkenylene group.

Examples of the arylene group for Y²⁰¹ include groups in which one hydrogen atom has been removed from an aryl group given as an example of the aromatic hydrocarbon group for R¹⁰¹ in the aforementioned formula (b-1).

Examples of the alkylene group and alkenylene group for Y²⁰¹ include groups in which one hydrogen atom has been removed from the chain-like alkyl group or the chain-like alkenyl group given as an example of R¹⁰¹ in the aforementioned formula (b-1).

In formulae (ca-4) and (ca-5), x represents 1 or 2.

W²⁰¹ represents a linking group having a valency of (x+1), i.e., a divalent or trivalent linking group.

As the divalent linking group for W²⁰¹, a divalent hydrocarbon group which may have a substituent is preferable, and as examples thereof, the same hydrocarbon groups (which may have a substituent) as those described above for Ya²¹ in the general formula (a2-1) may be mentioned. The divalent linking group for W²⁰¹ may be linear, branched or cyclic, and cyclic is more preferable. Among these, an arylene group having two carbonyl groups, each bonded to the terminal thereof is preferable. Examples of the arylene group include a phenylene group and a naphthylene group, and a phenylene group is particularly desirable.

As the trivalent linking group for W²⁰¹, a group in which one hydrogen atom has been removed from the aforementioned divalent linking group for W²⁰¹ and a group in which the divalent linking group has been bonded to another divalent linking group may be mentioned. The trivalent linking group for W²⁰¹ is preferably a group in which 2 carbonyl groups are bonded to an arylene group.

Specific examples of preferable cations represented by formula (ca-1) include cations represented by formulae (ca-1-1) to (ca-1-72) shown below.

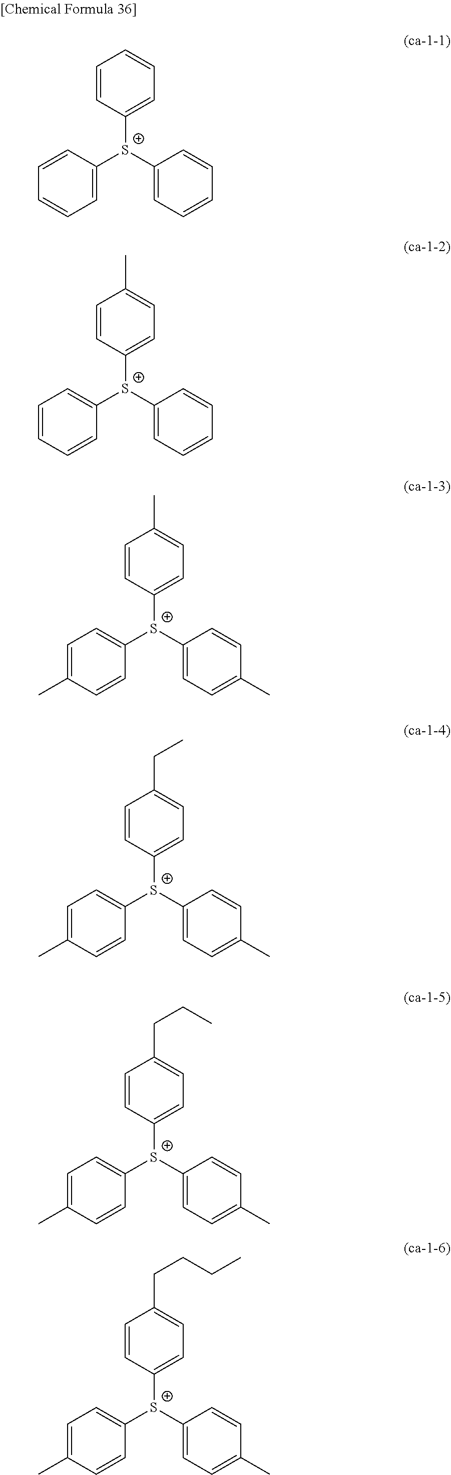

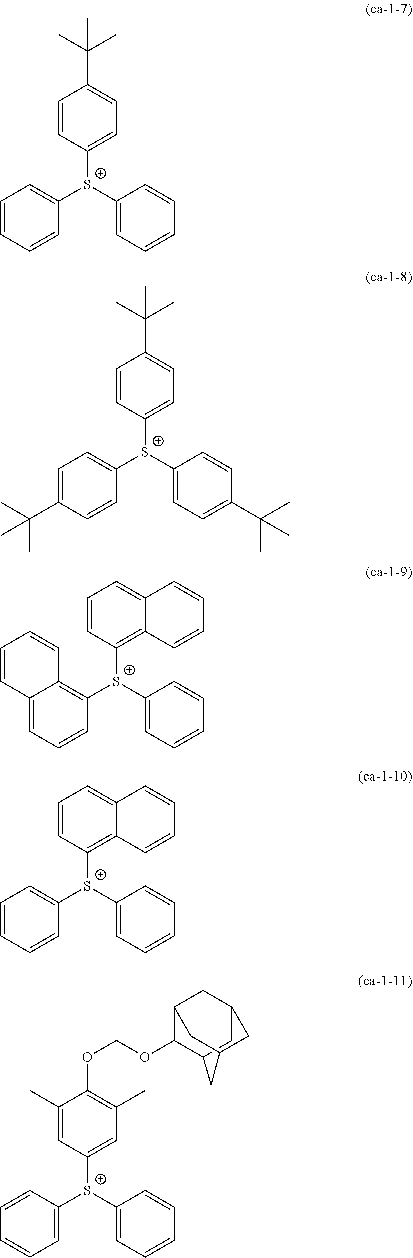

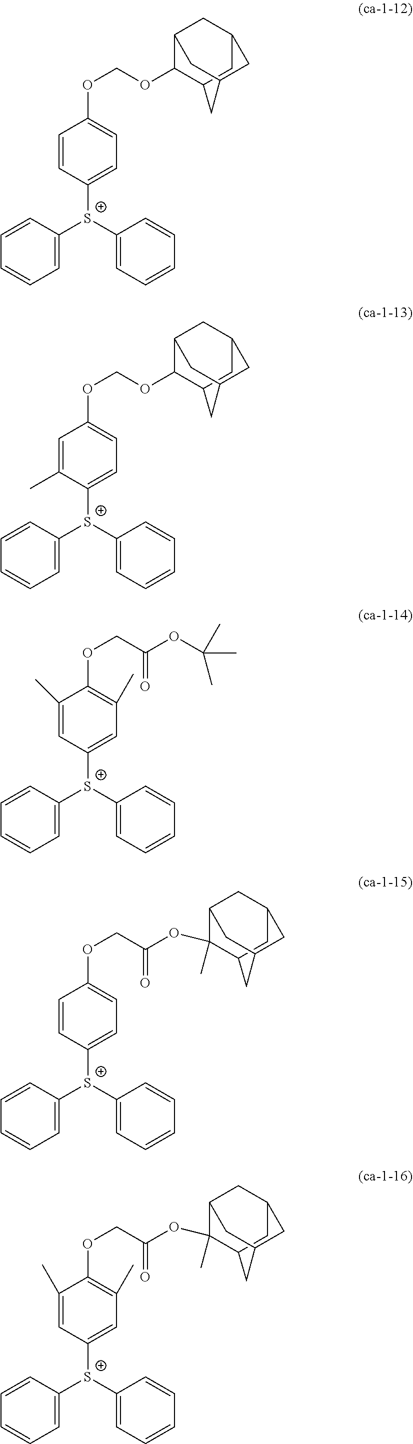

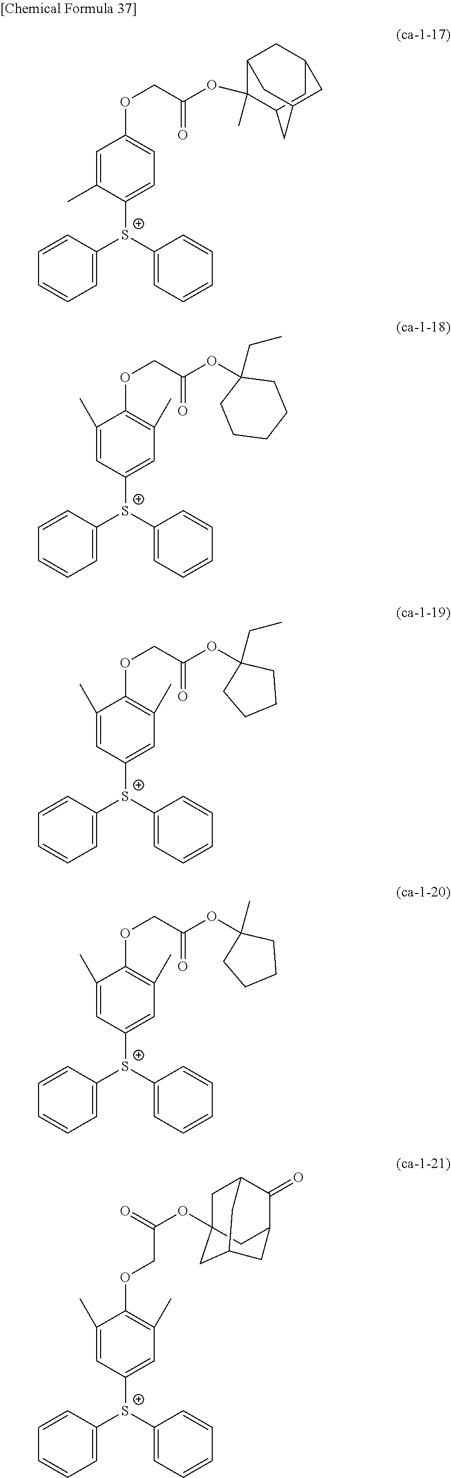

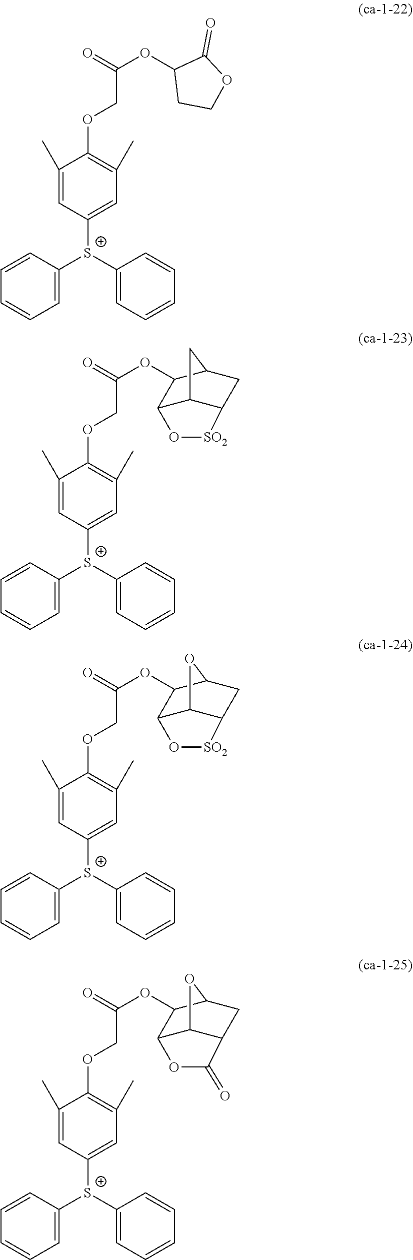

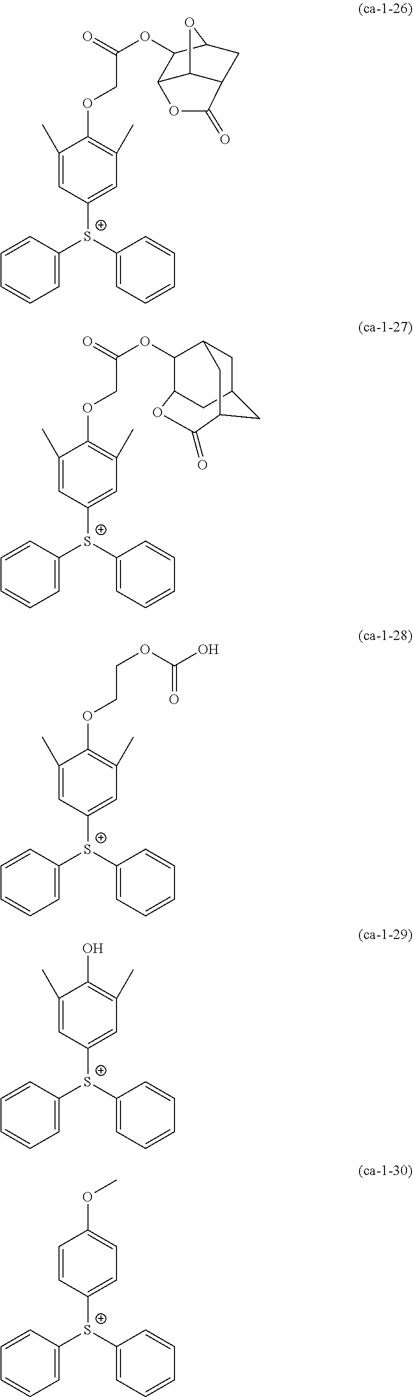

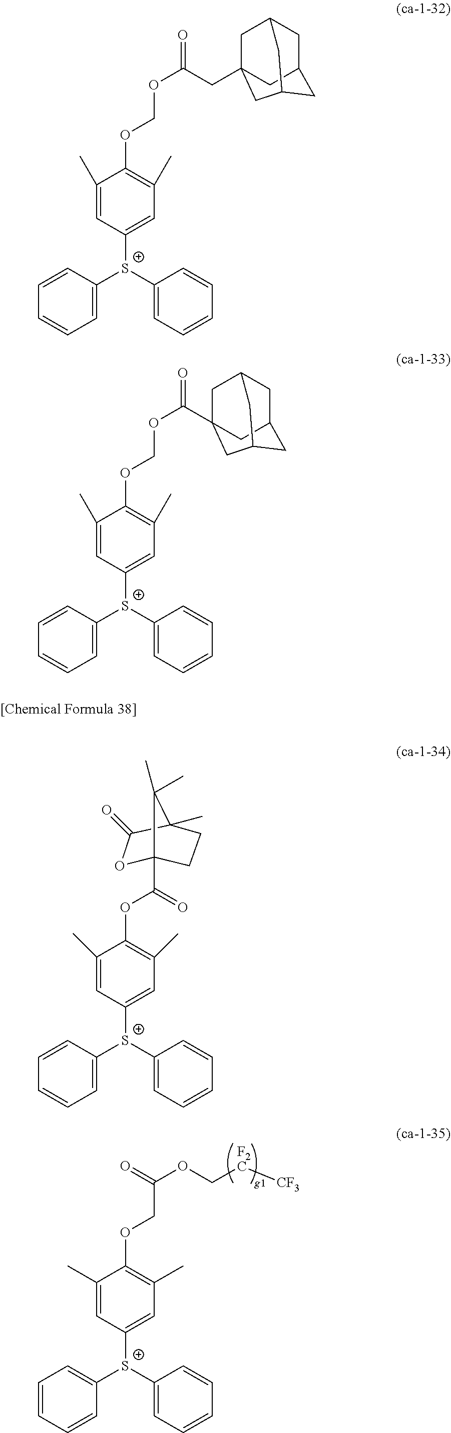

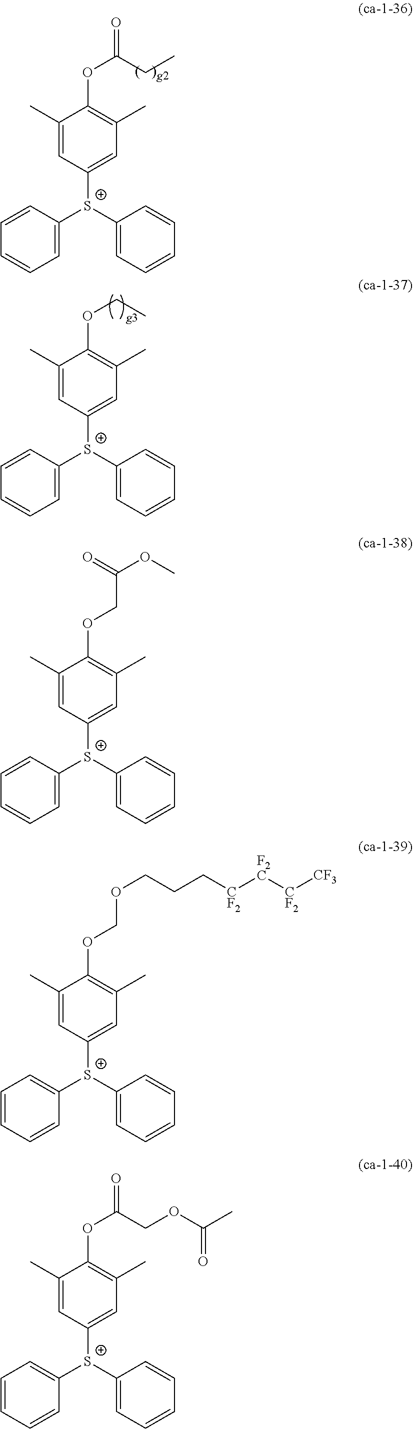

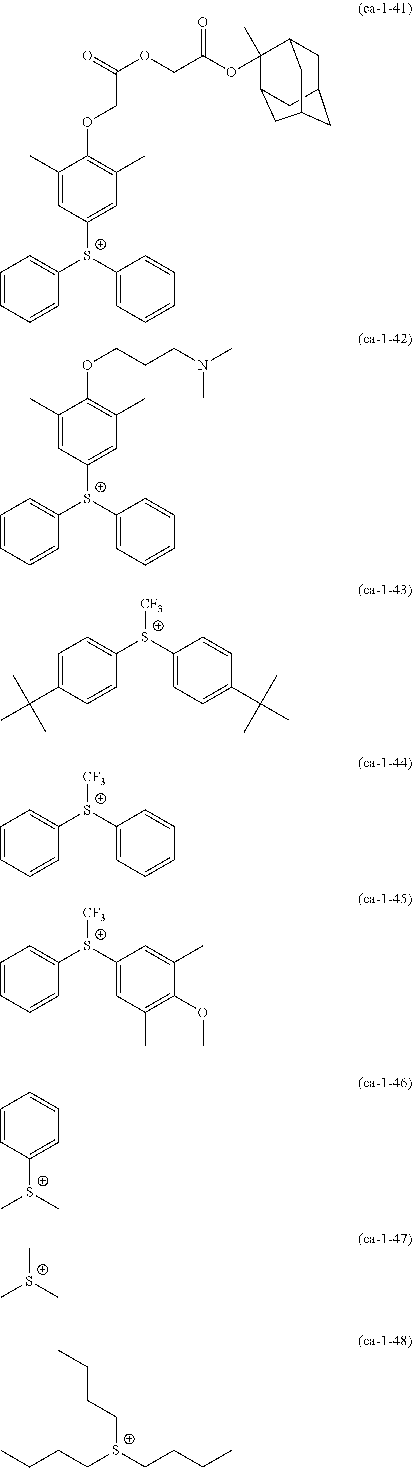

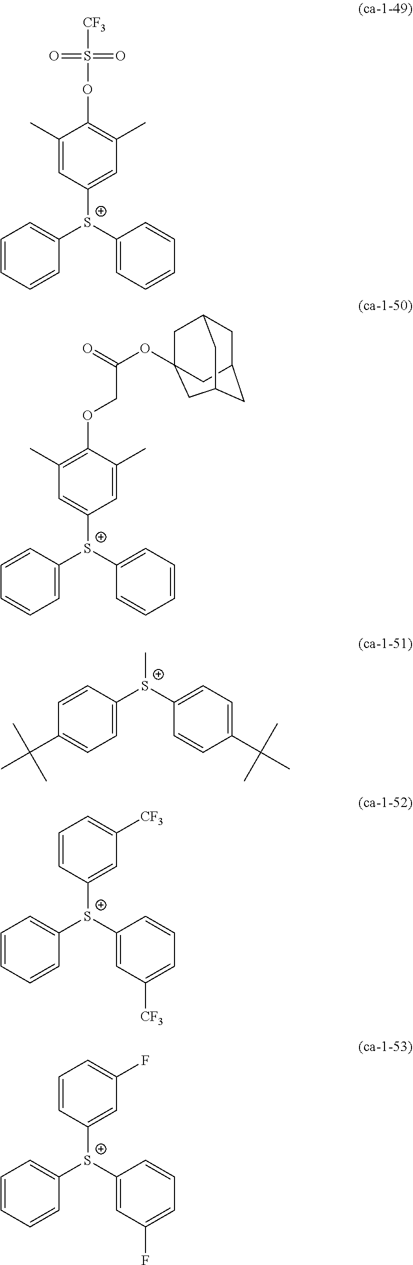

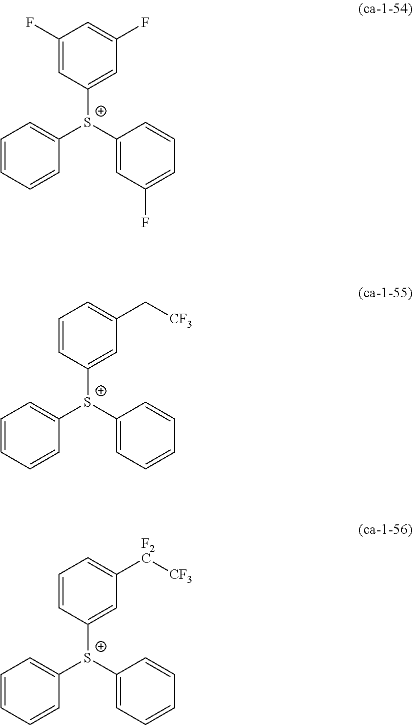

In the formulae, g1, g2 and g3 represent recurring numbers, wherein g1 is an integer of 1 to 5, g2 is an integer of 0 to 20, and g3 is an integer of 0 to 20.

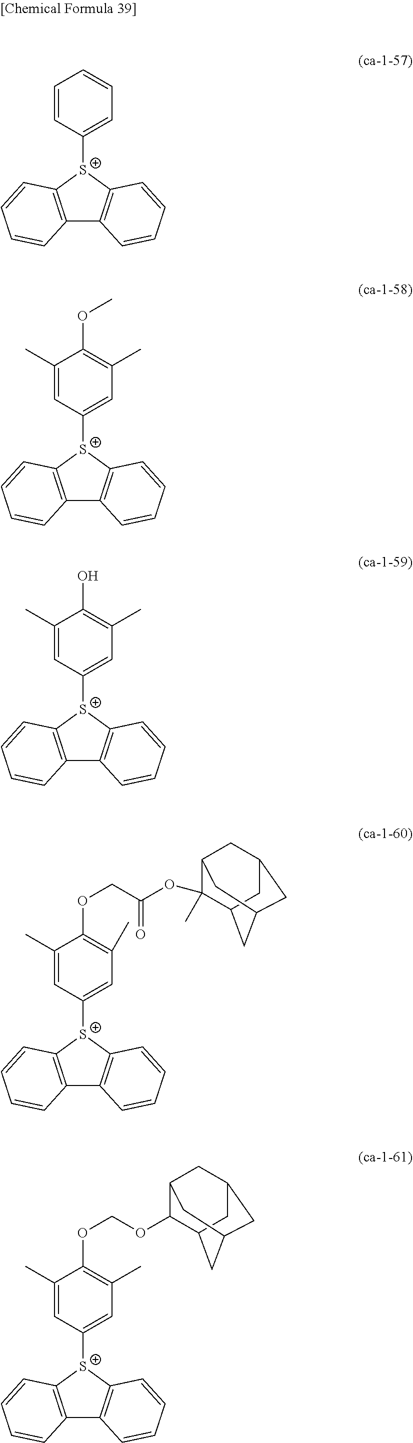

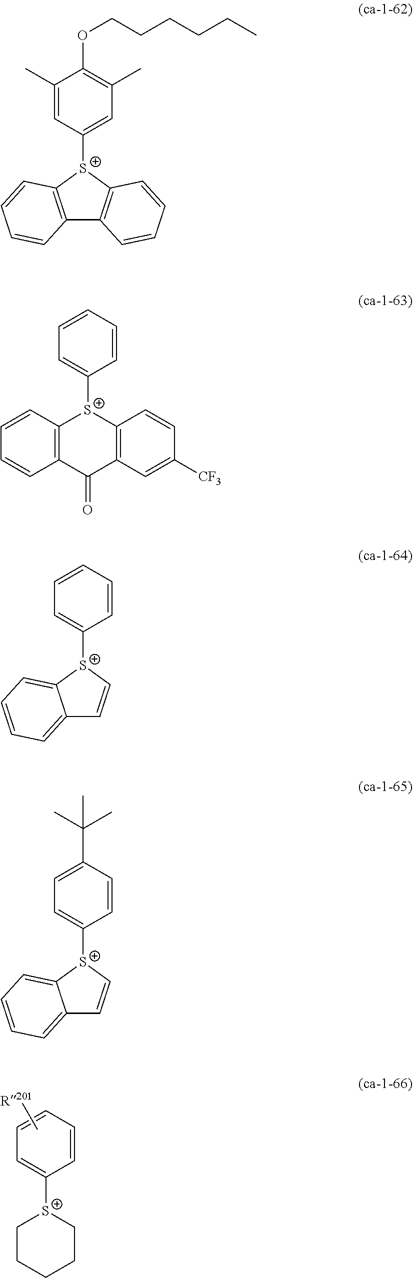

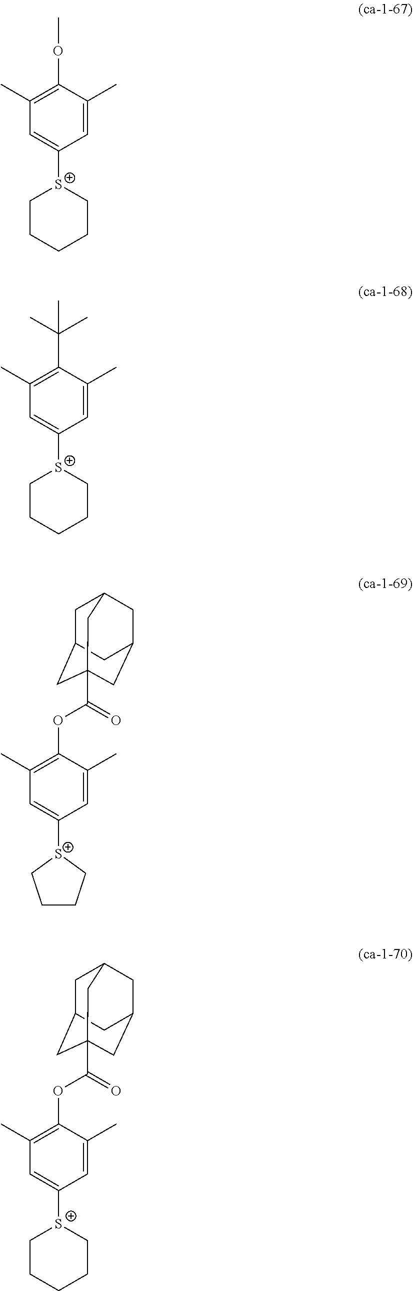

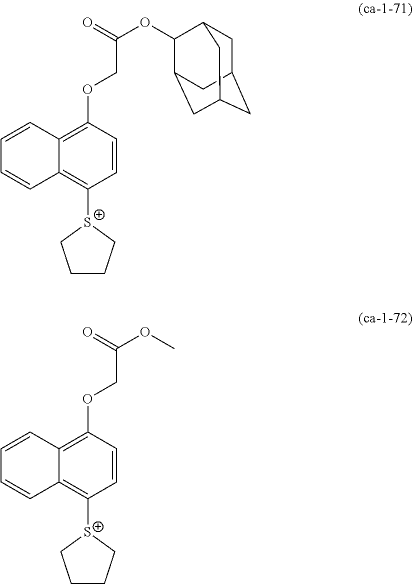

In the formulae, R″²⁰¹ represents a hydrogen atom or a substituent, and as the substituent, the same groups as those described above for substituting R²⁰¹ to R²⁰⁷ and R²¹⁰ to R²¹² may be mentioned.

Specific examples of preferable cations represented by the formula (ca-2) include a dihphenyliodonium cation and a bis(4-tert-butylphenyl)iodonium cation.

Specific examples of preferable cations represented by formula (ca-3) include cations represented by formulae (ca-3-1) to (ca-3-6) shown below.

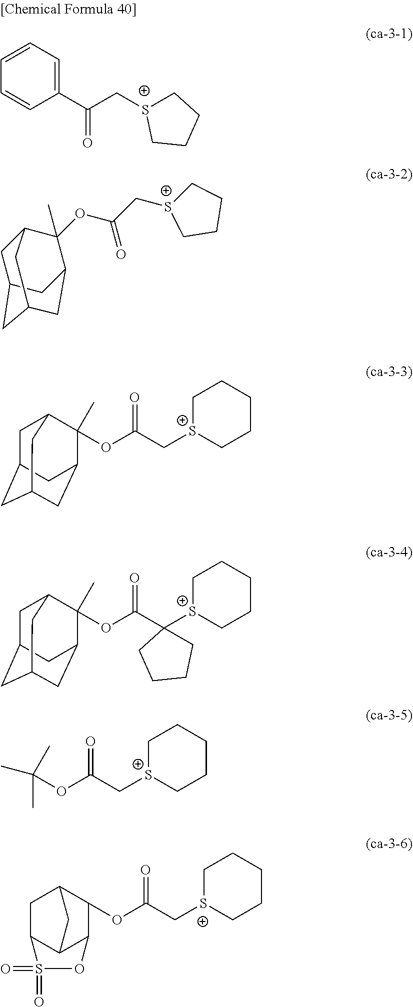

Specific examples of preferable cations represented by formula (ca-4) include cations represented by formulae (ca-4-1) and (ca-4-2) shown below.

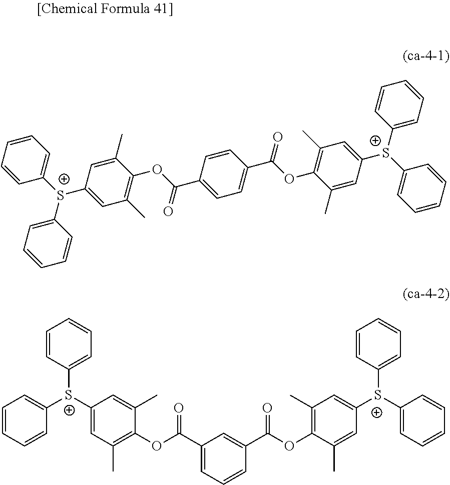 [SMILES: Cc1cc([S+](c2ccccc2)c2ccccc2)cc(C)c1OC(=O)c1ccc(C(=O)Oc2c(C)cc([S+](c3ccccc3)c3ccccc3)cc2C)cc1 ; Cc1cc([S+](c2ccccc2)c2ccccc2)cc(C)c1OC(=O)c1cccc(C(=O)Oc2c(C)cc([S+](c3ccccc3)c3ccccc3)cc2C)c1]

Further, examples of preferable cations represented by formula (ca-5) include cations represented by formulae (ca-5-1) to (ca-5-3) shown below.

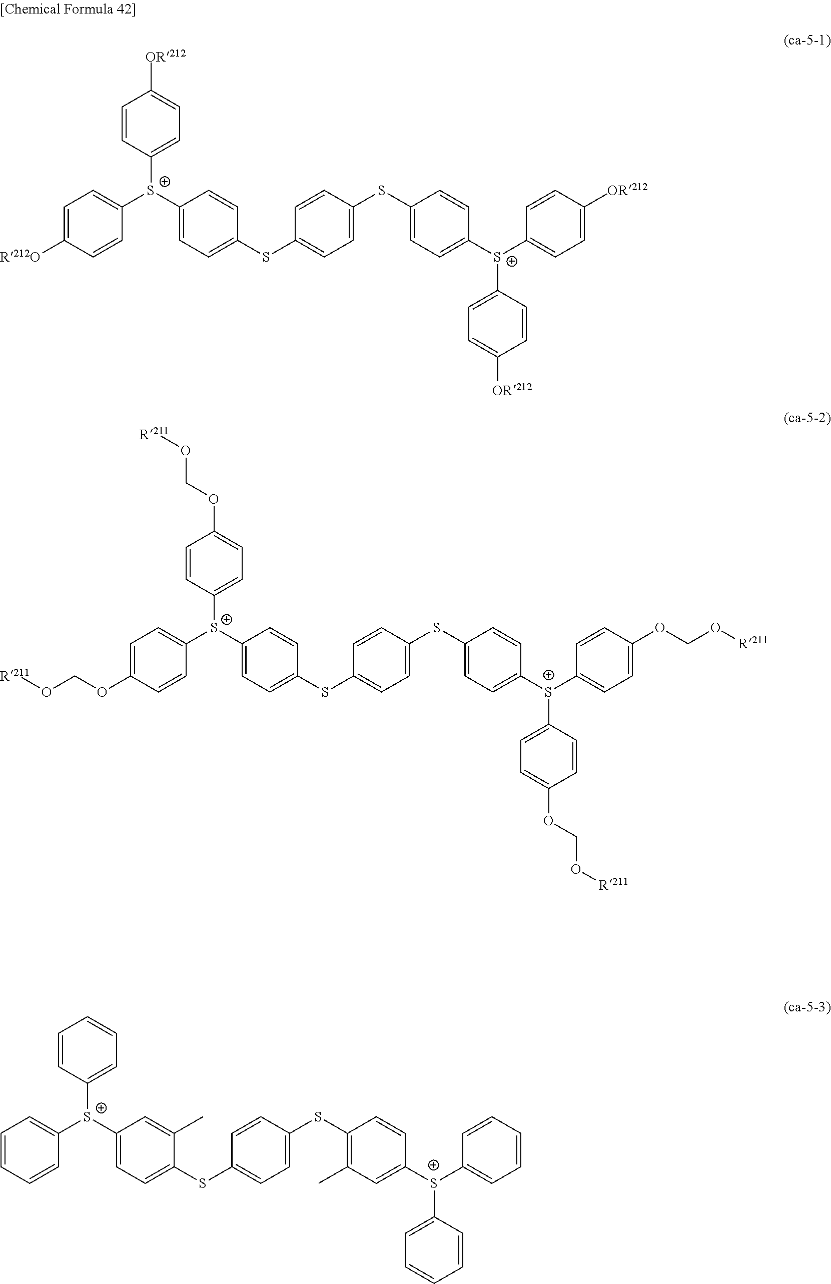

Among the above examples, as the cation moiety [(M′^{m+})_{1/m}], a cation represented by general formula (ca-1) is preferable, and a cation represented by any one of formulae (ca-1-1) to (ca-1-72) is more preferable.

As the component (B), one type of these acid generators may be used alone, or two or more types may be used in combination.

When the resist composition contains the component (B), the amount of the component (B) relative to 100 parts by weight of the component (A) is preferably within a range from 5 to 50 parts by weight, more preferably from 10 to 30 parts by weight, and still more preferably from 10 to 20 parts by weight.

When the amount of the component (B) is within the above-mentioned range, formation of a resist pattern may be satisfactorily performed. Further, by virtue of the above-mentioned range, when each of the components are dissolved in an organic solvent, a homogeneous solution may be more reliably obtained and the storage stability of the resist composition becomes satisfactory.

<<Acid Diffusion Control Agent (D)>>

The resist composition according to the present embodiment may include an acid diffusion control agent component (hereafter, sometimes referred to as “component (D)”), in addition to the component (A), or in addition to the component (A) and the component (B). The component (D) functions as an acid diffusion control agent, i.e., a quencher which traps the acid generated in the resist composition upon exposure.

The component (D) may be a photodecomposable base (D1) (hereafter, referred to as “component (D1)”) which is decomposed upon exposure and then loses the ability of controlling of acid diffusion, or a nitrogen-containing organic compound (D2) (hereafter, referred to as “component (D2)”) which does not fall under the definition of component (D1).

Component (D1)

When a resist pattern is formed using a resist composition containing the component (D1), the contrast between exposed portions and unexposed portions of the resist film is improved.

The component (D1) is not particularly limited, as long as it is decomposed upon exposure and then loses the ability of controlling of acid diffusion. As the component (D1), at least one compound selected from the group consisting of a compound represented by general formula (d1-1) shown below (hereafter, referred to as “component (d1-1)”), a compound represented by general formula (d1-2) shown below (hereafter, referred to as “component (d1-2)”) and a compound represented by general formula (d1-3) shown below (hereafter, referred to as “component (d1-3)”) is preferably used.

At exposed portions of the resist film, the components (d1-1) to (d1-3) are decomposed and then lose the ability of controlling of acid diffusion (i.e., basicity), and therefore the components (d1-1) to (d1-3) do not function as a quencher, whereas at unexposed portions, the components (d1-1) to (d1-3) functions as a quencher.

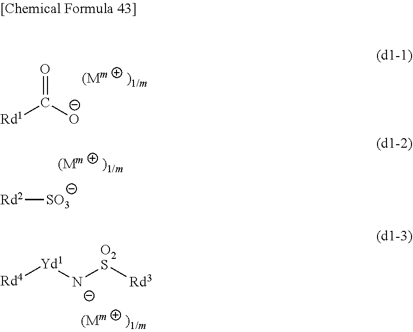

In the formulae, Rd¹ to Rd⁴ represent a cyclic group which may have a substituent, a chain alkyl group which may have a substituent or a chain alkenyl group which may have a substituent, provided that, the carbon atom adjacent to the sulfur atom within the Rd² in the formula (d1-2) has no fluorine atom bonded thereto; Yd¹ represents a single bond or a divalent linking group; m represents an integer of 1 or more; and each M^{m+} independently represents an organic cation having a valency of m.

{Component (d1-1)}

Anion Moiety

In formula (d1-1), Rd¹ represents a cyclic group which may have a substituent, a chain-like alkyl group which may have a substituent or a chain-like alkenyl group which may have a substituent, and is the same groups as those defined above for R¹⁰¹ in the aforementioned formula (b-1).

Among these, as the group for Rd¹, an aromatic hydrocarbon group which may have a substituent, an aliphatic cyclic group which may have a substituent and a chain-like alkyl group which may have a substituent are preferable. Examples of the substituent for these groups include a hydroxy group, an oxo group, an alkyl group, an aryl group, a fluorine atom, a fluorinated alkyl group, a lactone-containing cyclic group represented by any one of the aforementioned formulae (a2-r-1) to (a2-r-7), an ether bond, an ester bond, and a combination thereof. In the case where an ether bond or an ester bond is included as the substituent, the substituent may be bonded via an alkylene group, and a linking group represented by any one of the aforementioned formulae (y-a1-1) to (y-a1-5) is preferable as the substituent.

The aromatic hydrocarbon group is preferably a phenyl group or a naphthyl group.

Examples of the aliphatic cyclic group include groups in which one or more hydrogen atoms have been removed from a polycycloalkane such as adamantane, norbornane, isobornane, tricyclodecane or tetracyclododecane.

The chain-like alkyl group preferably has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl or a decyl group, and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group or a 4-methylpentyl group.

In the case where the chain-like alkyl group is a fluorinated alkyl group having a fluorine atom or a fluorinated alkyl group, the fluorinated alkyl group preferably has 1 to 11 carbon atoms, more preferably 1 to 8 carbon atoms, and still more preferably 1 to 4 carbon atoms. The fluorinated alkyl group may contain an atom other than fluorine. Examples of the atom other than fluorine include an oxygen atom, a sulfur atom and a nitrogen atom.

As Rd¹, a fluorinated alkyl group in which part or all of the hydrogen atoms constituting a linear alkyl group have been substituted with fluorine atom(s) is preferable, and a fluorinated alkyl group in which all of the hydrogen atoms constituting a linear alkyl group have been substituted with fluorine atoms (i.e., a linear perfluoroalkyl group) is particularly desirable.

Specific examples of preferable anion moieties for the component (d1-1) are shown below.

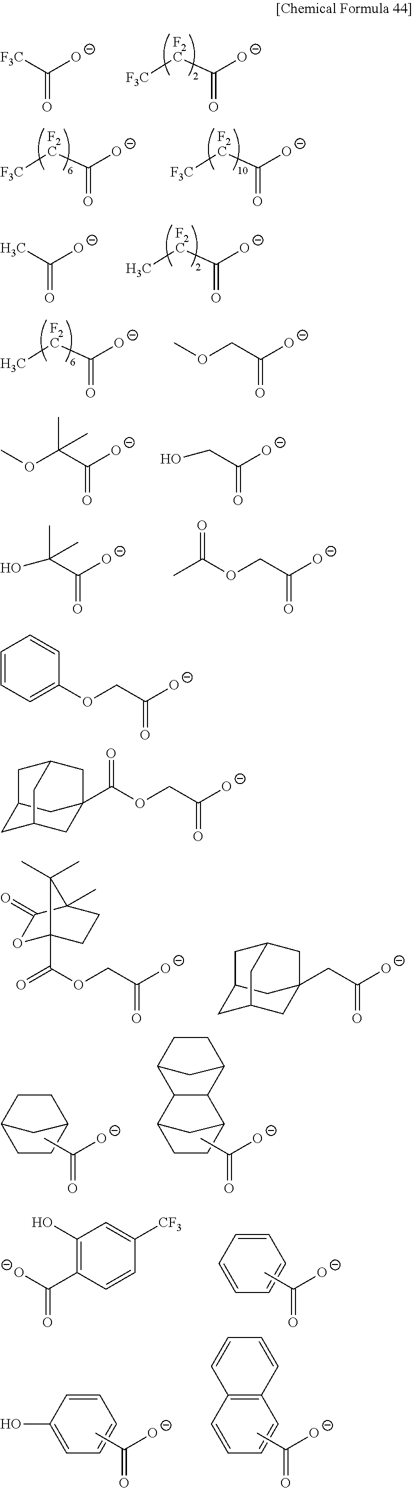

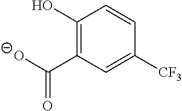 [SMILES: O=C([O-])c1cc(C(F)(F)F)ccc1O]

Cation Moiety

In formula (d1-1), M^{m+} represents an organic cation having a valency of m.

As the organic cation for M^{m+}, for example, the same cation moieties as those represented by the aforementioned formulae (ca-1) to (ca-5) are preferable, cation moieties represented by the aforementioned general formulae (ca-1) is preferable, and cation moieties represented by the aforementioned formulae (ca-1-1) to (ca-1-72) are still more preferable.

As the component (d1-1), one kind of compound may be used, or two or more kinds of compounds may be used in combination.

{Component (d1-2)}

Anion Moiety

In formula (d1-2), Rd² represents a cyclic group which may have a substituent, a chain-like alkyl group which may have a substituent or a chain-like alkenyl group which may have a substituent, and is the same groups as those defined above for R¹⁰¹ in the aforementioned formula (b-1).

provided that, the carbon atom adjacent to the sulfur atom within Rd² group has no fluorine atom bonded thereto (i.e., the carbon atom adjacent to the sulfur atom within Rd² group does not substituted with a fluorine atom). As a result, the anion of the component (d1-2) becomes an appropriately weak acid anion, thereby improving the quenching ability of the component (D).

As Rd², a chain-like alkyl group which may have a substituent or an aliphatic cyclic group which may have a substituent is preferable. The chain-like alkyl group preferably has 1 to 10 carbon atoms, and more preferably 3 to 10 carbon atoms. As the aliphatic cyclic group, a group in which one or more hydrogen atoms have been removed from adamantane, norbornane, isobornane, tricyclodecane, tetracyclododecane or camphor (which may have a substituent) is more preferable.

The hydrocarbon group for Rd² may have a substituent. As the substituent, the same groups as those described above for substituting the hydrocarbon group (e.g., aromatic hydrocarbon group, aliphatic cyclic group, chain-like alkyl group) for Rd¹ in the formula (d1-1) may be mentioned.

Specific examples of preferable anion moieties for the component (d1-2) are shown below.

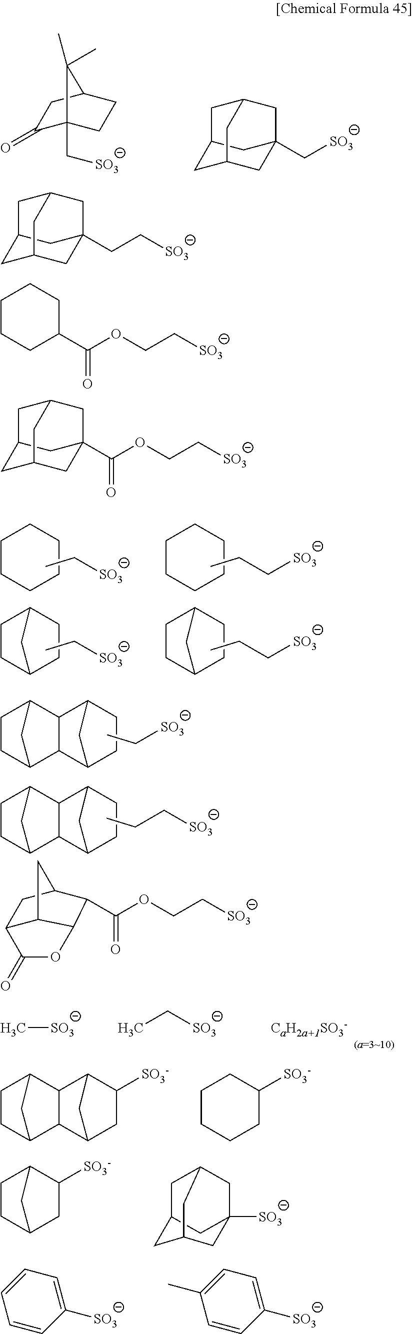

Cation Moiety

In formula (d1-2), M^{m+} is an organic cation having a valency of m, and is the same as defined for M^{m+} in the aforementioned formula (d1-1).

As the component (d1-2), one type of compound may be used, or two or more types of compounds may be used in combination.

{Component (d1-3)}

Anion Moiety

In formula (d1-3), Rd³ represents a cyclic group which may have a substituent, a chain-like alkyl group which may have a substituent or a chain-like alkenyl group which may have a substituent, and is the same groups as those defined above for R¹⁰¹ in the aforementioned formula (b-1), and a cyclic group containing a fluorine atom, a chain-like alkyl group or a chain-like alkenyl group is preferable. Among these, a fluorinated alkyl group is preferable, and more preferably the same fluorinated alkyl groups as those described above for Rd¹.

In formula (d1-3), Rd⁴ represents a cyclic group which may have a substituent, a chain-like alkyl group which may have a substituent or a chain-like alkenyl group which may have a substituent, and is the same groups as those defined above for R¹⁰¹ in the aforementioned formula (b-1).

Among these, an alkyl group which may have substituent, an alkoxy group which may have substituent, an alkenyl group which may have substituent or a cyclic group which may have substituent is preferable.

The alkyl group for Rd⁴ is preferably a linear or branched alkyl group of 1 to 5 carbon atoms, and specific examples include a methyl group, an ethyl group, a propyl group, an isopropyl group, an n-butyl group, an isobutyl group, a tert-butyl group, a pentyl group, an isopentyl group, and a neopentyl group. Part of the hydrogen atoms within the alkyl group for Rd⁴ may be substituted with a hydroxy group, a cyano group or the like.

The alkoxy group for Rd⁴ is preferably an alkoxy group of 1 to 5 carbon atoms, and specific examples thereof include a methoxy group, an ethoxy group, an n-propoxy group, an iso-propoxy group, an n-butoxy group and a tert-butoxy group. Among these, a methoxy group and an ethoxy group are preferable.

As the alkenyl group for Rd⁴, the same groups as those described above for R¹⁰¹ in the aforementioned formula (b-1) may be mentioned, and a vinyl group, a propenyl group (an allyl group), a 1-methylpropenyl group and a 2-methylpropenyl group are preferable. These groups may have an alkyl group of 1 to 5 carbon atoms or a halogenated alkyl group of 1 to 5 carbon atoms as a substituent.

As the cyclic group for Rd⁴, the same groups as those described above for R¹⁰¹ in the aforementioned formula (b-1) may be mentioned. Among these, as the cyclic group, an alicyclic group (e.g., a group in which one or more hydrogen atoms have been removed from a cycloalkane such as cyclopentane, cyclohexane, adamantane, norbornane, isobornane, tricyclodecane or tetracyclododecane) or an aromatic group (e.g., a phenyl group or a naphthyl group) is preferable. When Rd⁴ is an alicyclic group, the resist composition may be satisfactorily dissolved in an organic solvent, thereby improving the lithography properties. Alternatively, when Rd⁴ is an aromatic group, the resist composition exhibits an excellent photoabsorption efficiency in a lithography process using EUV or the like as the exposure source, thereby resulting in the improvement of the sensitivity and the lithography properties.

In formula (d1-3), Yd¹ represents a single bond or a divalent linking group. The divalent linking group for Yd¹ is not particularly limited, and examples thereof include a divalent hydrocarbon group (aliphatic hydrocarbon group, or aromatic hydrocarbon group) which may have a substituent and a divalent linking group containing a hetero atom. The divalent linking groups are the same as defined for the divalent hydrocarbon group which may have a substituent and the divalent linking group containing a hetero atom explained above as the divalent linking group for Ya²¹ in the aforementioned formula (a2-1).

As Yd¹, a carbonyl group, an ester bond, an amide bond, an alkylene group or a combination of these is preferable. As the alkylene group, a linear or branched alkylene group is more preferable, and a methylene group or an ethylene group is still more preferable.

Specific examples of preferable anion moieties for the component (d1-3) are shown below.

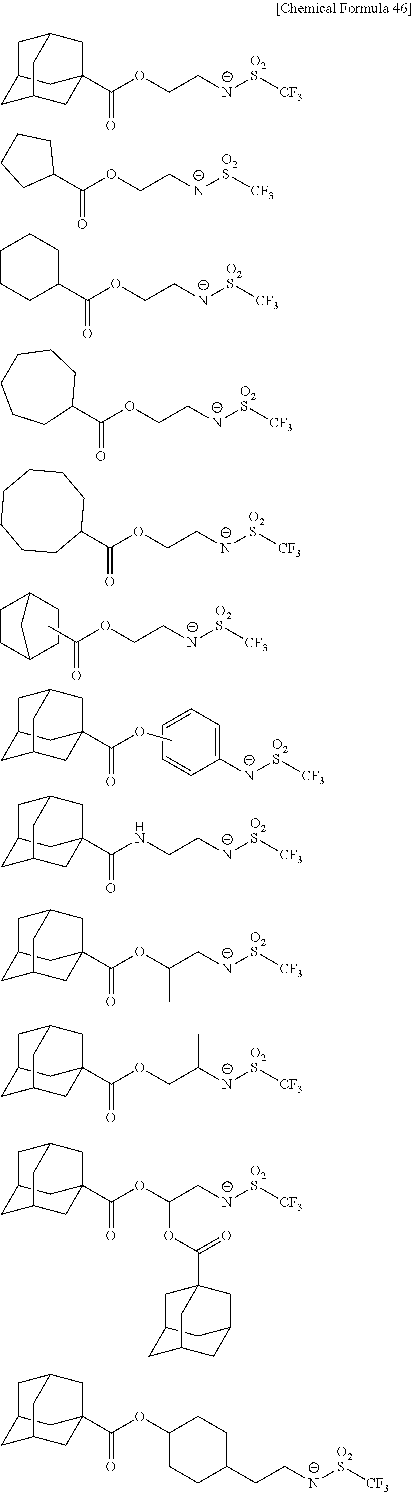

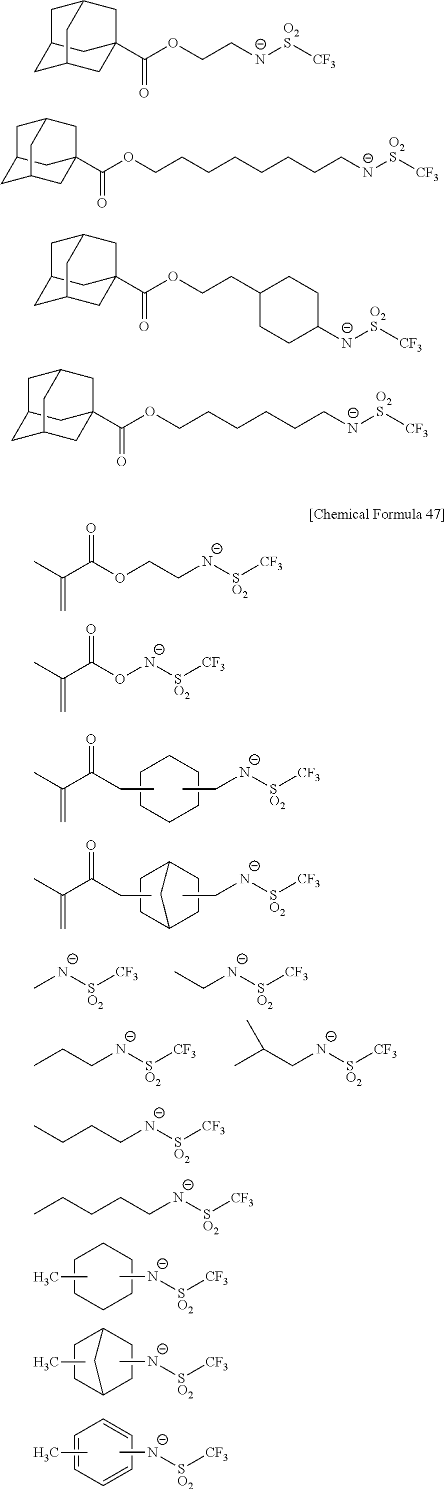

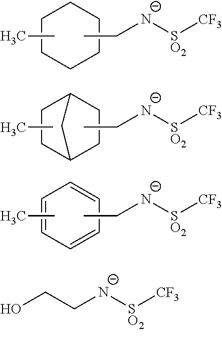

Cation Moiety

In formula (d1-3), M^{m+} is an organic cation having a valency of m, and is the same as defined for M^{m+} in the aforementioned formula (d1-1).

As the component (d1-3), one type of compound may be used, or two or more types of compounds may be used in combination.

As the component (D1), one kind of the aforementioned components (d1-1) to (d1-3), or at least two kinds of the aforementioned components (d1-1) to (d1-3) may be used in combination.

When the resist composition contains the component (D1), the amount of the component (D1) relative to 100 parts by weight of the component (A) is preferably within a range from 0.5 to 10 parts by weight, more preferably from 0.5 to 8 parts by weight, and still more preferably from 1 to 8 parts by weight.

When the amount of the component (D1) is at least as large as the lower limit of the above-mentioned range, excellent lithography properties and excellent resist pattern shape may be more reliably obtained. On the other hand, when the amount of the component (D1) is no more than the upper limit of the above-mentioned range, sensitivity may be maintained at a satisfactory level, and through-put becomes excellent.

Production method of component (D1):

The production methods of the components (d1-1) and (d1-2) are not particularly limited, and the components (d1-1) and (d1-2) may be produced by conventional methods.

Further, the production method of the component (d1-3) is not particularly limited, and the component (d1-3) may be produced in the same manner as disclosed in US2012-0149916.

Component (D2)

The acid diffusion control component may contain a nitrogen-containing organic compound (D2) (hereafter, referred to as component (D2)) which does not fall under the definition of component (D1).

The component (D2) is not particularly limited, as long as it functions as an acid diffusion control agent, and does not fall under the definition of the component (D1). As the component (D2), any of the conventionally known compounds may be selected for use. Among these, an aliphatic amine is preferable, and a secondary aliphatic amine or tertiary aliphatic amine is more preferable.

An aliphatic amine is an amine having one or more aliphatic groups, and the aliphatic groups preferably have 1 to 12 carbon atoms.

Examples of these aliphatic amines include amines in which at least one hydrogen atom of ammonia (NH₃) has been substituted with an alkyl group or hydroxyalkyl group of no more than 12 carbon atoms (i.e., alkylamines or alkylalcoholamines), and cyclic amines.

Specific examples of alkylamines and alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-hexylamine, tri-n-pentylamine, tri-n-heptylamine, tri-n-octylamine, tri-n-nonylamine, tri-n-decylamine, and tri-n-dodecylamine; and alkyl alcohol amines such as diethanolamine, triethanolamine, diisopropanolamine, triisopropanolamine, di-n-octanolamine, and tri-n-octanolamine. Among these, trialkylamines of 5 to 10 carbon atoms are preferable, and tri-n-pentylamine and tri-n-octylamine are particularly desirable.

Examples of the cyclic amine include heterocyclic compounds containing a nitrogen atom as a hetero atom. The heterocyclic compound may be a monocyclic compound (aliphatic monocyclic amine), or a polycyclic compound (aliphatic polycyclic amine).

Specific examples of the aliphatic monocyclic amine include piperidine, and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine and triethanolamine triacetate, and triethanolamine triacetate is preferable.

Further, as the component (D2), an aromatic amine may be used.

Examples of aromatic amines include 4-dimethylaminopyridine, pyrrole, indole, pyrazole, imidazole and derivatives thereof, as well as tribenzylamine, 2,6-diisopropylaniline and N-tert-butoxycarbonylpyrrolidine.

As the component (D2), one type of compound may be used alone, or two or more types may be used in combination.

When the resist composition contains the component (D2), the amount of the component (D2) is typically used in an amount within a range from 0.01 to 5 parts by weight, relative to 100 parts by weight of the component (A). When the amount of the component (D) is within the above-mentioned range, the shape of the resist pattern and the post exposure stability of the latent image formed by the pattern-wise exposure of the resist layer are improved.

<<At Least One Compound (E) Selected from the Group Consisting of an Organic Carboxylic Acid, or a Phosphorus Oxo Acid or Derivative Thereof>>

In the resist composition of the present embodiment, for preventing any deterioration in sensitivity, and improving the resist pattern shape and the post exposure stability of the latent image formed by the pattern-wise exposure of the resist layer, at least one compound (E) (hereafter referred to as the component (E)) selected from the group consisting of an organic carboxylic acid, or a phosphorus oxo acid or derivative thereof may be added.

Examples of suitable organic carboxylic acids include acetic acid, malonic acid, citric acid, malic acid, succinic acid, benzoic acid, and salicylic acid.

Examples of phosphorus oxo acids include phosphoric acid, phosphonic acid and phosphinic acid. Among these, phosphonic acid is particularly desirable.

Examples of oxo acid derivatives include esters in which a hydrogen atom within the above-mentioned oxo acids is substituted with a hydrocarbon group. Examples of the hydrocarbon group include an alkyl group of 1 to 5 carbon atoms and an aryl group of 6 to 15 carbon atoms.

Examples of phosphoric acid derivatives include phosphoric acid esters such as di-n-butyl phosphate and diphenyl phosphate.

Examples of phosphonic acid derivatives include phosphonic acid esters such as dimethyl phosphonate, di-n-butyl phosphonate, phenylphosphonic acid, diphenyl phosphonate and dibenzyl phosphonate.

Examples of phosphinic acid derivatives include phosphinic acid esters and phenylphosphinic acid.

As the component (E), one type may be used alone, or two or more types may be used in combination.

When the resist composition contains the component (E), the amount of the component (E) is typically used in an amount within a range from 0.01 to 5 parts by weight, relative to 100 parts by weight of the component (A).

<<Fluorine Additive (F)>>

In the present embodiment, the resist composition may further include a fluorine additive (hereafter, referred to as “component (F)”) for imparting water repellency to the resist film.

As the component (F), for example, a fluorine-containing polymeric compound described in Japanese Unexamined Patent Application, First Publication No. 2010-002870, Japanese Unexamined Patent Application, First Publication No. 2010-032994, Japanese Unexamined Patent Application, First Publication No. 2010-277043, Japanese Unexamined Patent Application, First Publication No. 2011-13569, and Japanese Unexamined Patent Application, First Publication No. 2011-128226 may be used.

Specific examples of the component (F) include polymers having a structural unit (f1) represented by general formula (f1-1) shown below. As the polymer, a polymer (homopolymer) consisting of a structural unit (f1) represented by formula (f1-1) shown below; a copolymer of the structural unit (f1) and a structural unit (a1) containing an acid decomposable group that exhibits increased polarity by the action of acid; and a copolymer of the structural unit (f1), a structural unit derived from acrylic acid or methacrylic acid and the structural unit (a1) are preferable. As the structural unit (a1) to be copolymerized with the structural unit (f1), a structural unit derived from 1-ethyl-1-cyclooctyl (meth)acrylate or a structural unit derived from 1-methyl-1-adamantyl (meth)acrylate is preferable.

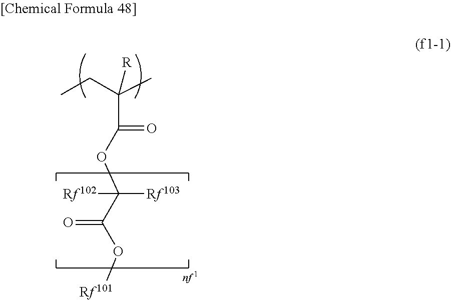

In the formula, R is the same as defined above; Rf¹⁰² and Rf¹⁰³ each independently represents a hydrogen atom, a halogen atom, an alkyl group of 1 to 5 carbon atoms, or a halogenated alkyl group of 1 to 5 carbon atoms, provided that Rf¹⁰² and Rf¹⁰³ may be the same or different; nf¹ represents an integer of 1 to 5; and Rf¹⁰¹ represents an organic group containing a fluorine atom.

In formula (f1-1), R bonded to the carbon atom on the α-position is the same as defined above. As R, a hydrogen atom or a methyl group is preferable.

In formula (f1-1), examples of the halogen atom for Rf¹⁰² and Rf¹⁰³ include a fluorine atom, a chlorine atom, a bromine atom and an iodine atom, and a fluorine atom is particularly desirable. Examples of the alkyl group of 1 to 5 carbon atoms for Rf¹⁰² and Rf¹⁰³ include the same alkyl group of 1 to 5 carbon atoms as those described above for R, and a methyl group or an ethyl group is preferable. Specific examples of the halogenated alkyl group of 1 to 5 carbon atoms represented by Rf¹⁰² or Rf¹⁰³ include groups in which part or all of the hydrogen atoms of the aforementioned alkyl groups of 1 to 5 carbon atoms have been substituted with halogen atoms.

Examples of the halogen atom include a fluorine atom, a chlorine atom, a bromine atom and an iodine atom, and a fluorine atom is particularly desirable. Among these examples, as Rf¹⁰² and Rf¹⁰³, a hydrogen atom, a fluorine atom or an alkyl group of 1 to 5 carbon atoms is preferable, and a hydrogen atom, a fluorine atom, a methyl group or an ethyl group is more preferable.

In formula (f1-1), nf¹ represents an integer of 1 to 5, preferably an integer of 1 to 3, and more preferably 1 or 2.

In formula (f1-1), Rf¹⁰¹ represents an organic group containing a fluorine atom, and is preferably a hydrocarbon group containing a fluorine atom.

The hydrocarbon group containing a fluorine atom may be linear, branched or cyclic, and preferably has 1 to 20 carbon atoms, more preferably 1 to 15 carbon atoms, and most preferably 1 to 10 carbon atoms.

It is preferable that the hydrocarbon group having a fluorine atom has 25% or more of the hydrogen atoms within the hydrocarbon group fluorinated, more preferably 50% or more, and most preferably 60% or more, as the hydrophobicity of the resist film during immersion exposure is enhanced.

Among these, as Rf¹⁰¹, a fluorinated hydrocarbon group of 1 to 6 carbon atoms is preferable, and a trifluoromethyl group, —CH₂—CF₃, —CH₂—CF₂—CF₃, —CH(CF₃)₂, —CH₂—CH₂—CF₃, and —CH₂—CH₂—CF₂—CF₂—CF₂—CF₃ are most preferable.

The weight average molecular weight (Mw) (the polystyrene equivalent value determined by gel permeation chromatography) of the component (F) is preferably 1,000 to 50,000, more preferably 5,000 to 40,000, and most preferably 10,000 to 30,000. When the Mw of the component (F) is no more than the upper limit of the above-mentioned range, the resist composition exhibits a satisfactory solubility in a resist solvent. On the other hand, when the Mw is at least as large as the lower limit of the above-mentioned range, dry etching resistance and the cross-sectional shape of the resist pattern becomes satisfactory.

Further, the dispersity (Mw/Mn) of the component (F) is preferably 1.0 to 5.0, more preferably 1.0 to 3.0, and most preferably 1.2 to 2.5.

As the component (F), one type may be used alone, or two or more types may be used in combination.

When the resist composition contains the component (F), the component (F) is used in an amount within a range from 0.5 to 10 parts by weight, relative to 100 parts by weight of the component (A).

If desired, other miscible additives may also be added to the resist composition of the present invention. Examples of such miscible additives include additive resins for improving the performance of the resist film, dissolution inhibitors, plasticizers, stabilizers, colorants, halation prevention agents, and dyes.

<<Organic Solvent (S)>>

The resist composition of the present embodiment may be prepared by dissolving the resist materials for the resist composition in an organic solvent (hereafter, referred to as “component (S)”).

The component (S) may be any organic solvent which can dissolve the respective components to give a homogeneous solution, and any organic solvent may be appropriately selected from those which have been conventionally known as solvents for a chemically amplified resist composition.

Examples of the component (S) include lactones such as γ-butyrolactone; ketones such as acetone, methyl ethyl ketone, cyclohexanone, methyl-n-pentyl ketone, methyl isopentyl ketone, and 2-heptanone; polyhydric alcohols, such as ethylene glycol, diethylene glycol, propylene glycol and dipropylene glycol; compounds having an ester bond, such as ethylene glycol monoacetate, diethylene glycol monoacetate, propylene glycol monoacetate, and dipropylene glycol monoacetate; polyhydric alcohol derivatives including compounds having an ether bond, such as a monoalkylether (e.g., monomethylether, monoethylether, monopropylether or monobutylether) or monophenylether of any of these polyhydric alcohols or compounds having an ester bond (among these, propylene glycol monomethyl ether acetate (PGMEA) and propylene glycol monomethyl ether (PGME) are preferable); cyclic ethers such as dioxane; esters such as methyl lactate, ethyl lactate (EL), methyl acetate, ethyl acetate, butyl acetate, methyl pyruvate, ethyl pyruvate, methyl methoxypropionate, and ethyl ethoxypropionate; aromatic organic solvents such as anisole, ethylbenzylether, cresylmethylether, diphenylether, dibenzylether, phenetole, butylphenylether, ethylbenzene, diethylbenzene, pentylbenzene, isopropylbenzene, toluene, xylene, cymene and mesitylene; and dimethylsulfoxide (DMSO).

The component (S) may be used individually, or in combination as a mixed solvent.

Among these, PGMEA, PGME, γ-butyrolactone, EL and cyclohexanone are preferable.

Further, among the mixed solvents, a mixed solvent obtained by mixing PGMEA with a polar solvent is preferable. The mixing ratio (weight ratio) of the mixed solvent may be appropriately determined, taking into consideration the compatibility of the PGMEA with the polar solvent, but is preferably in the range of 1:9 to 9:1, more preferably from 2:8 to 8:2.

Specifically, when EL or cyclohexanone is mixed as the polar solvent, the PGMEA:EL or cyclohexanone weight ratio is preferably from 1:9 to 9:1, and more preferably from 2:8 to 8:2. Alternatively, when PGME is mixed as the polar solvent, the PGMEA:PGME weight ratio is preferably from 1:9 to 9:1, more preferably from 2:8 to 8:2, and still more preferably 3:7 to 7:3. Furthermore, a mixed solvent of PGMEA, PGME and cyclohexanone is also preferable.

Further, as the component (S), a mixed solvent of at least one of PGMEA and EL with γ-butyrolactone is also preferable. The mixing ratio (former:latter) of such a mixed solvent is preferably from 70:30 to 95:5.

The amount of the component (S) is not particularly limited, and is appropriately adjusted to a concentration which enables coating of a coating solution to a substrate. In general, the component (S) is used in an amount such that the solid content of the resist composition becomes within the range from 1 to 20% by weight, and preferably from 2 to 15% by weight.

As described above, in the resist composition of the present embodiment, a resin component (A1) having a structural unit (a0) represented by general formula (a0-1) is employed.

The side chain of the structural unit (a0) has a partial structure (linker portion) represented by —Ya⁰¹-C(Ra⁰¹)(Ra⁰²)—Ya⁰²-. Since the linker portion has an alkyl chain of an appropriate length, the polarity of the structural unit (a0) may be reduced without lowering the Tg of the structural unit (a0). Therefore, in the resist composition of the present embodiment containing the resin component (A1) having the structural unit (a0), the solubility of the resist composition in a developer containing an organic solvent may be improved, while maintaining high resistance to a rinse solution of an alcohol solvent such as methyl isobutyl carbinol (MIBC).

In addition, the resist composition of the present embodiment has high resistance to a rinse solution and improved solubility in a developing solution containing an organic solvent. Therefore, when the resist composition of the present embodiment is applied to a solvent developing process, it is considered that a resist pattern having improved lithography properties such as CDU may be formed.

(Method of Forming a Resist Pattern)

The method of forming a resist pattern according to the second aspect of the present invention includes: using a resist composition according to the first aspect to form a resist film on a substrate; exposing the resist film; and developing the exposed resist film using a developing solution containing an organic solvent to form a resist pattern.

The method for forming a resist pattern may be performed, for example, by the following embodiment.

In the method of forming a resist pattern according to the present embodiment, firstly, a resist composition of the first aspect is applied to a substrate using a spinner or the like, and a bake treatment (post applied bake (PAB)) is conducted at a temperature of 80 to 150° C. for 40 to 120 seconds, preferably 60 to 90 seconds, to form a resist film.

Following selective exposure of the thus formed resist film, either by exposure through a mask having a predetermined pattern formed thereon (mask pattern) using an exposure apparatus such as an ArF exposure apparatus, an electron beam lithography apparatus or an EUV exposure apparatus, or by patterning via direct irradiation with an electron beam without using a mask pattern, baking treatment (post exposure baking (PEB)) is conducted under temperature conditions of 80 to 150° C. for 40 to 120 seconds, and preferably 60 to 90 seconds.

Next, the resist film is subjected to a developing treatment. The developing treatment is conducted using a developing solution containing an organic solvent (organic developing solution).

After the developing treatment, it is preferable to conduct a rinse treatment. The rinse treatment is preferably conducted using a rinse solution containing an organic solvent.

After the developing treatment or the rinse treatment, a process of removing the developing solution or the rinsing solution attached to the pattern with a supercritical fluid may be performed.

After the developing treatment or the rinse treatment, drying is conducted. If desired, bake treatment (post bake) may be conducted following the developing.

In this manner, a resist pattern can be formed.

The substrate is not specifically limited and a conventionally known substrate may be used. For example, substrates for electronic components, and such substrates having wiring patterns formed thereon may be used. Specific examples of the material of the substrate include metals such as silicon wafer, copper, chromium, iron and aluminum; and glass. Suitable materials for the wiring pattern include copper, aluminum, nickel, and gold.

Further, as the substrate, any one of the above-mentioned substrates provided with an inorganic and/or organic film on the surface thereof may be used. As the inorganic film, an inorganic antireflection film (inorganic BARC) may be used. As the organic film, an organic antireflection film (organic BARC) and an organic film such as a lower-layer organic film used in a multilayer resist method may be used.

Here, a “multilayer resist method” is method in which at least one layer of an organic film (lower-layer organic film) and at least one layer of a resist film (upper resist film) are provided on a substrate, and a resist pattern formed on the upper resist film is used as a mask to conduct patterning of the lower-layer organic film. This method is considered as being capable of forming a pattern with a high aspect ratio. More specifically, in the multilayer resist method, a desired thickness may be ensured by the lower-layer organic film, and as a result, the thickness of the resist film may be reduced, and an extremely fine pattern with a high aspect ratio may be formed.

The multilayer resist method is broadly classified into a method in which a double-layer structure consisting of an upper-layer resist film and a lower-layer organic film is formed (double-layer resist method), and a method in which a multilayer structure having at least three layers consisting of an upper-layer resist film, a lower-layer organic film and at least one intermediate layer (thin metal film or the like) provided between the upper-layer resist film and the lower-layer organic film (triple-layer resist method).

The wavelength to be used for exposure is not particularly limited and the exposure may be conducted using radiation such as ArF excimer laser, KrF excimer laser, F₂ excimer laser, extreme ultraviolet rays (EUV), vacuum ultraviolet rays (VUV), electron beam (EB), X-rays, and soft X-rays. The resist composition of the present invention is effective to KrF excimer laser, ArF excimer laser, EB and EUV, and particularly effective to ArF excimer laser, EB and EUV.

The exposure of the resist film may be either a general exposure (dry exposure) conducted in air or an inert gas such as nitrogen, or immersion exposure (immersion lithography).

In immersion lithography, the region between the resist film and the lens at the lowermost point of the exposure apparatus is pre-filled with a solvent (immersion medium) that has a larger refractive index than the refractive index of air, and the exposure (immersion exposure) is conducted in this state.

The immersion medium preferably exhibits a refractive index larger than the refractive index of air but smaller than the refractive index of the resist film to be exposed. The refractive index of the immersion medium is not particularly limited as long as it satisfies the above-mentioned requirements.

Examples of this immersion medium which exhibits a refractive index that is larger than the refractive index of air but smaller than the refractive index of the resist film include water, fluorine-based inert liquids, silicon-based solvents and hydrocarbon-based solvents.

Specific examples of the fluorine-based inert liquids include liquids containing a fluorine-based compound such as C₃HCl₂F₅, C₄F₉OCH₃, C₄F₉OC₂H₅ or C₅H₃F₇ as the main component, which have a boiling point within a range from 70 to 180° C. and preferably from 80 to 160° C. A fluorine-based inert liquid having a boiling point within the above-mentioned range is advantageous in that the removal of the immersion medium after the exposure may be conducted by a simple method.

As a fluorine-based inert liquid, a perfluoroalkyl compound in which all of the hydrogen atoms of the alkyl group are substituted with fluorine atoms is particularly desirable. Examples of these perfluoroalkyl compounds include perfluoroalkylether compounds and perfluoroalkylamine compounds.

Specifically, one example of a suitable perfluoroalkylether compound is perfluoro(2-butyl-tetrahydrofuran) (boiling point 102° C.), and an example of a suitable perfluoroalkylamine compound is perfluorotributylamine (boiling point 174° C.).

As the immersion medium, water is preferable in terms of cost, safety, environment and versatility.

The organic solvent contained in the organic developer may be any as long as it can dissolve the component (A) (the component (A) before exposure), and may be appropriately selected from known organic solvents. Specific examples of the organic solvent include polar solvents such as ketone solvents, ester solvents, alcohol solvents, nitrile solvents, amide solvents and ether solvents, and hydrocarbon solvents.

A ketone solvent is an organic solvent containing C—C(═O)—C within the structure thereof. An ester solvent is an organic solvent containing C—C(═O)—O—C within the structure thereof. An alcohol solvent is an organic solvent containing an alcoholic hydroxy group in the structure thereof. An “alcoholic hydroxy group” refers to a hydroxy group bonded to a carbon atom of an aliphatic hydrocarbon group. A nitrile solvent is an organic solvent containing a nitrile group in the structure thereof. An amide solvent is an organic solvent containing an amide group within the structure thereof. An ether solvent is an organic solvent containing C—O—C within the structure thereof.

Some organic solvents have a plurality of the functional groups which characterizes the aforementioned solvents within the structure thereof. In such a case, the organic solvent may be classified as any type of the solvent having the characteristic functional group. For example, diethyleneglycol monomethylether may be classified as either an alcohol solvent or an ether solvent.

A hydrocarbon solvent consists of a hydrocarbon which may be halogenated, and does not have any substituent other than a halogen atom. Examples of the halogen atom include a fluorine atom, a chlorine atom, a bromine atom and an iodine atom, and a fluorine atom is preferable.

As the organic solvent contained in the organic developing solution, among these, a polar solvent is preferable, and ketone solvents, ester solvents and nitrile solvents are preferable.

Examples of ketone solvents include 1-octanone, 2-octanone, 1-nonanone, 2-nonanone, acetone, 4-heptanone, 1-hexanone, 2-hexanone, diisobutyl ketone, cyclohexanone, methylcyclohexanone, phenylacetone, methyl ethyl ketone, methyl isobutyl ketone, acetylacetone, acetonylacetone, ionone, diacetonylalcohol, acetylcarbinol, acetophenone, methyl naphthyl ketone, isophorone, propylenecarbonate, γ-butyrolactone and methyl amyl ketone (2-heptanone). Among these examples, as a ketone solvent, methyl amyl ketone (2-heptanone) is preferable.

Examples of ester solvents include methyl acetate, butyl acetate, ethyl acetate, isopropyl acetate, amyl acetate, isoamyl acetate, ethyl methoxyacetate, ethyl ethoxyacetate, propylene glycol monomethyl ether acetate, ethylene glycol monoethyl ether acetate, ethylene glycol monopropyl ether acetate, ethylene glycol monobutyl ether acetate, ethylene glycol monophenyl ether acetate, diethylene glycol monomethyl ether acetate, diethylene glycol monopropyl ether acetate, diethylene glycol monoethyl ether acetate, diethylene glycol monophenyl ether acetate, diethylene glycol monobutyl ether acetate, diethylene glycol monoethyl ether acetate, 2-methoxybutyl acetate, 3-methoxybutyl acetate, 4-methoxybutyl acetate, 3-methyl-3-methoxybutyl acetate, 3-ethyl-3-methoxybutyl acetate, propylene glycol monomethyl ether acetate, propylene glycol monoethyl ether acetate, propylene glycol monopropyl ether acetate, 2-ethoxybutyl acetate, 4-ethoxybutyl acetate, 4-propoxybutyl acetate, 2-methoxypentyl acetate, 3-methoxypentyl acetate, 4-methoxypentyl acetate, 2-methyl-3-methoxypentyl acetate, 3-methyl-3-methoxypentyl acetate, 3-methyl-4-methoxypentyl acetate, 4-methyl-4-methoxypentyl acetate, propylene glycol diacetate, methyl formate, ethyl formate, butyl formate, propyl formate, ethyl lactate, butyl lactate, propyl lactate, ethyl carbonate, propyl carbonate, butyl carbonate, methyl pyruvate, ethyl pyruvate, propyl pyruvate, butyl pyruvate, methyl acetoacetate, ethyl acetoacetate, methyl propionate, ethyl propionate, propyl propionate, isopropyl propionate, methyl 2-hydroxypropionate, ethyl 2-hydroxypropionate, methyl-3-methoxypropionate, ethyl-3-methoxypropionate, ethyl-3-ethoxypropionate and propyl-3-methoxypropionate. Among these examples, as an ester solvent, butyl acetate is preferable.

Examples of nitrile solvents include acetonitrile, propionitrile, valeronitrile, butyronitrile and the like.

If desired, the organic developing solution may have a conventional additive blended. Examples of the additive include surfactants. The surfactant is not particularly limited, and for example, an ionic or non-ionic fluorine and/or silicon surfactant may be used.

As the surfactant, a non-ionic surfactant is preferable, and a non-ionic fluorine surfactant or a non-ionic silicon surfactant is more preferable.

When a surfactant is added, the amount thereof based on the total amount of the organic developing solution is generally 0.001 to 5% by weight, preferably 0.005 to 2% by weight, and more preferably 0.01 to 0.5% by weight.

The developing treatment may be performed by a conventional developing method. Examples thereof include a method in which the substrate is immersed in the developing solution for a predetermined time (a dip method), a method in which the developing solution is cast up on the surface of the substrate by surface tension and maintained for a predetermined period (a puddle method), a method in which the developing solution is sprayed onto the surface of the substrate (spray method), and a method in which the developing solution is continuously ejected from a developing solution ejecting nozzle while scanning at a constant rate to apply the developing solution to the substrate while rotating the substrate at a constant rate (dynamic dispense method).

As the organic solvent contained in the rinse solution used for the rinse treatment after the development treatment, for example, among the organic solvents mentioned as the organic solvent used for the organic developer, those which hardly dissolve the resist pattern may be appropriately selected for use. In general, at least one solvent selected from the group consisting of hydrocarbon solvents, ketone solvents, ester solvents, alcohol solvents, amide solvents and ether solvents is used. Among these, at least one solvent selected from the group consisting of hydrocarbon solvents, ketone solvents, ester solvents, alcohol solvents and amide solvents is preferable, more preferably at least one solvent selected from the group consisting of alcohol solvents and ester solvents, and an alcohol solvent is particularly desirable.

The alcohol solvent used for the rinse liquid is preferably a monohydric alcohol of 6 to 8 carbon atoms, and the monohydric alcohol may be linear, branched or cyclic. Specific examples thereof include 1-hexanol, 1-heptanol, 1-octanol, 2-hexanol, 2-heptanol, 2-octanol, 3-hexanol, 3-heptanol, 3-octanol, 4-octanol and benzyl alcohol. Among these, 1-hexanol, 2-heptanol and 2-hexanol are preferable, and 1 hexanol and 2-hexanol are more preferable.

As the organic solvent, one kind of solvent may be used alone, or two or more kinds of solvents may be used in combination. Further, an organic solvent other than the aforementioned examples or water may be mixed together. However, in consideration of the development characteristics, the amount of water within the rinse liquid, based on the total amount of the rinse liquid is preferably 30% by weight or less, more preferably 10% by weight or less, still more preferably 5% by weight or less, and most preferably 3% by weight or less.

If desired, the rinse solution may have a conventional additive blended. Examples of the additive include surfactants. Examples of the additive include surfactants. As the surfactant, the same surfactants as those described above may be mentioned, a non-ionic surfactant is preferable, and a non-ionic fluorine surfactant or a non-ionic silicon surfactant is more preferable.

When a surfactant is added, the amount thereof based on the total amount of the rinse liquid is generally 0.001 to 5% by weight, preferably 0.005 to 2% by weight, and more preferably 0.01 to 0.5% by weight.

The rinse treatment using a rinse liquid (washing treatment) may be conducted by a conventional rinse method. Examples of the rinse method include a method in which the rinse liquid is continuously applied to the substrate while rotating it at a constant rate (rotational coating method), a method in which the substrate is immersed in the rinse liquid for a predetermined time (dip method), and a method in which the rinse liquid is sprayed onto the surface of the substrate (spray method).

According to the method of forming a resist pattern of the present embodiment described above, since the resist composition according to the first aspect described above is used, a pattern having good lithography properties such as CDU may be formed.

(Polymeric Compound)

A third aspect of the present invention is a polymeric compound having a structural unit (a0) represented by general formula (a0-1) shown below.

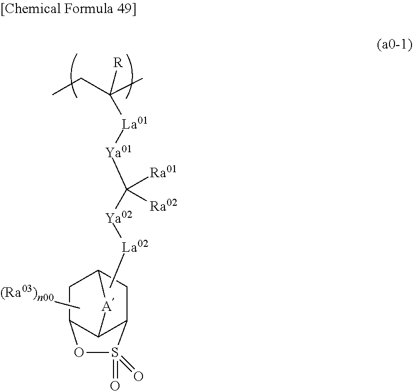

In the formula, R represents a hydrogen atom, an alkyl group of 1 to 5 carbon atoms or a halogenated alkyl group of 1 to 5 carbon atoms; La⁰¹ and La⁰² each independently represents a single bond or a divalent linking group;

Ya⁰¹ and Ya⁰² each independently represents a single bond or an alkylene group of 1 to 3 carbon atoms;

provided that the total number of carbon atoms of Ya⁰¹ and Ya⁰² is 3 or less; Ra⁰¹ and Ra⁰² each independently represents a hydrogen atom or an alkyl group of 1 to 4 carbon atoms; Ra⁰¹ and Ra⁰² may be mutually bonded to form a ring; provided that the total number of carbon atoms of Ra⁰¹ and Ra⁰² is 2 to 6; Ra⁰³ represents a hydrogen atom, an alkyl group, an alkoxy group, a halogen atom, a halogenated alkyl group, a hydroxy group, —COOR″, —OC(═O)R″, a hydroxyalkyl group or a cyano group; R″ represents a hydrogen atom, an alkyl group, a lactone-containing cyclic group, a carbonate-containing cyclic group or a —SO₂-containing cyclic group; n₀₀ represents an integer of 0 to 2;

when n₀₀ is 2, the plurality of Ra⁰³ may be the same or different from each other; and A′ represents an alkylene group of 1 to 5 carbon atoms.

The polymeric compound according to the present embodiment is the same as the component (A1) in the explanation of the “resist composition” described above, and the kinds of other structural units, the amount of each structural unit within the component (A1-1), and the like is the same as described above.

The polymeric compound of the present embodiment may be produced, for example, by dissolving a monomer for deriving the structural unit (a0) and, if necessary, a monomer for deriving another structural unit in a polymerization solvent, and adding a radical polymerization initiator such as azobisisobutyronitrile (AIBN) or dimethyl azobisisobutyrate (V-601) to effect polymerization.

The polymeric compound according to the present embodiment is a novel substance useful as a base resin for a resist composition. The polymeric compound may be preferably added to a resist composition as a base component having a film forming ability, and also as a resin component (component (A1)) which exhibits changed solubility in a developing solution by the action of acid.

(Compound)

A fourth aspect of the present invention is a compound represented by general formula (m-a0) shown below.

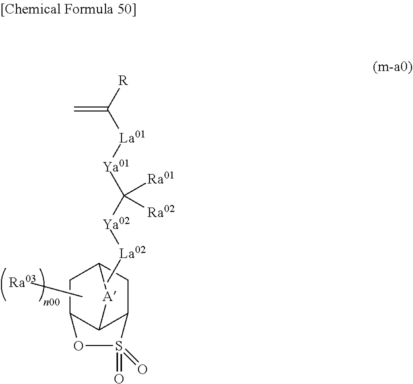

In the formula, R represents a hydrogen atom, an alkyl group of 1 to 5 carbon atoms or a halogenated alkyl group of 1 to 5 carbon atoms; La⁰¹ and La⁰² each independently represents a single bond or a divalent linking group;

Ya⁰¹ and Ya⁰² each independently represents a single bond or an alkylene group of 1 to 3 carbon atoms;

provided that the total number of carbon atoms of Ya⁰¹ and Ya⁰² is 3 or less; Ra⁰¹ and Ra⁰² each independently represents a hydrogen atom or an alkyl group of 1 to 4 carbon atoms; Ra⁰¹ and Ra⁰² may be mutually bonded to form a ring; provided that the total number of carbon atoms of Ra⁰¹ and Ra⁰² is 2 to 6; Ra⁰³ represents a hydrogen atom, an alkyl group, an alkoxy group, a halogen atom, a halogenated alkyl group, a hydroxy group, —COOR″, —OC(═O)R″, a hydroxyalkyl group or a cyano group; R″ represents a hydrogen atom, an alkyl group, a lactone-containing cyclic group, a carbonate-containing cyclic group or a —SO₂-containing cyclic group; n₀₀ represents an integer of 0 to 2;

when n₀₀ is 2, the plurality of Ra⁰¹ may be the same or different from each other; and A′ represents an alkylene group of 1 to 5 carbon atoms.

In formula (m-a0), R, La⁰¹, La⁰², Ya⁰¹, Ya⁰², Ra⁰¹, Ra⁰², Ra⁰³, n₀₀ and A′ are the same as defined for R, La⁰¹, La⁰², Ya⁰¹, Ya⁰², Ra⁰¹, Ra⁰², Ra⁰³, n₀₀ and A′ in the aforementioned formula (a0-1).

The compound of the present embodiment is preferably a compound represented by general formula (m-a0-1) shown below (hereafter, sometimes referred to as “compound (m-a0-1)”).

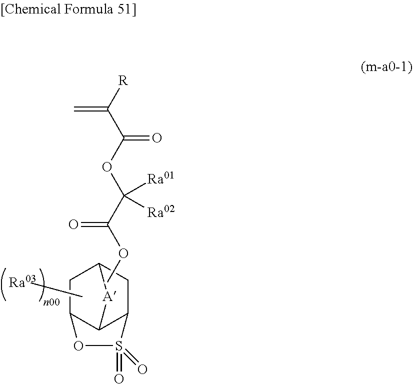

In the formula, R, Ra⁰¹, Ra⁰², Ra⁰³, n₀₀ and A′ are the same as defined for R, Ra⁰¹, Ra⁰², Ra⁰¹, n₀₀ and A′ in the aforementioned general formula (a0-1), respectively.

[Production Method of Compound (m-a0-1)]

The compound (m-a0-1) may be produced, for example, by reacting a compound (X-1) represented by general formula (X-1) shown below with a compound (X-2) represented by general formula (X-2) shown below in a solvent such as tetrahydrofuran, toluene or methylene chloride.

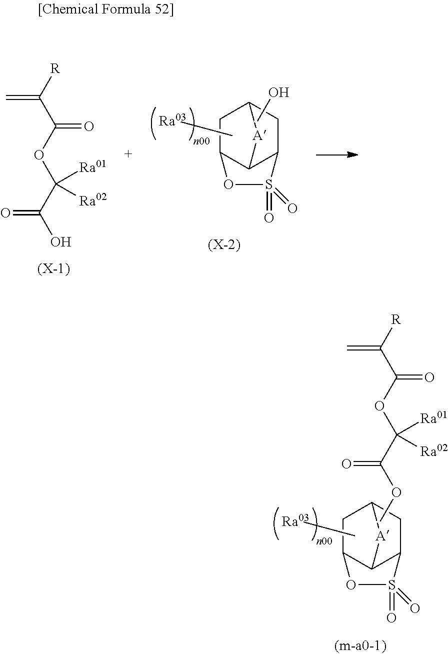

In the formula, R, Ra⁰¹, Ra⁰², Ra⁰³, n₀₀ and A′ are the same as defined for R, Ra⁰¹, Ra⁰², Ra⁰³, n₀₀ and A′ in the aforementioned general formula (a0-1), respectively.

For example, a reactive derivative of (meth)acrylic acid such as compound (X-1) may be reacted with a compound (X-2) in a solvent in the presence of a base such as triethylamine, pyridine, or 4-dimethylaminopyridine if desired to obtain a monomer represented by general formula (m-a0-1) (compound (m-a0-1)).

The compound (m-a0-1) may also be obtained by reacting the compound (X-2) with an ester of the compound (X-1) in a solvent in the presence of a transesterification catalyst such as titanium isopropoxide.

Alternatively, the compound (m-a0-1) may be obtained by reacting the compound (X-2) with the compound (X-1) in the presence of a strong acid such as hydrochloric acid, sulfuric acid, or p-toluenesulfonic acid.

The compound (m-a0-1) thus obtained may be separated and purified, for example, by separation means such as filtration, concentration, distillation, extraction, crystallization, recrystallization, column chromatography, or a combination thereof.

### EXAMPLES

As follows is a description of examples of the present invention, although the scope of the present invention is by no way limited by these examples.

### Synthesis Example 1: Synthesis of Polymer (1)

13.39 g of methyl ethyl ketone (MEK) was added to a three-necked flask connected with a thermometer, a reflux tube, and a nitrogen inlet tube, followed by heating to 87° C.

Then, 12.00 g (34.85 mmol) of a compound (m01) and 11.46 g (54.50 mmol) of a compound (m11) were dissolved in 34.24 g of MEK, and 0.782 g (3.40 mmol) of dimethyl 2,2′-azobis(isobutyrate) (V-601) as a polymerization initiator was dissolved therein. The resulting solution was dropwise added to the three-necked flask in a nitrogen atmosphere over 4 hours. Thereafter, the reaction solution was heated for 1 hour while stirring, and then cooled to room temperature.

The obtained reaction polymer solution was dropwise added to an excess amount of methanol to precipitate a polymer. Thereafter, the precipitated white powder was washed with MeOH/MEK in an excess amount (20 wt % or more), followed by drying, so as to obtain 16.05 g of polymer (1) as an objective compound (yield: 68.4%).

With respect to the polymer, the weight average molecular weight (Mw) and the dispersity (Mw/Mn) were determined by the polystyrene equivalent value as measured by gel permeation chromatography (GPC). As a result, it was found that the weight average molecular weight was 10,600, and the dispersity was 1.47.

Further, as a result of an analysis by carbon 13 nuclear magnetic resonance spectroscopy (600 MHz, ¹³C-NMR), it was found that the composition of the copolymer (ratio (molar ratio) of the respective structural units within the structural formula) was 42/58.

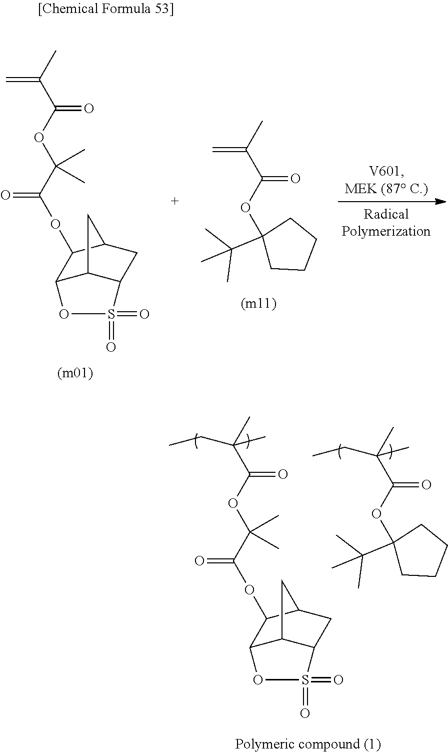

### Synthesis Examples 2 to 11: Synthesis of Polymeric Compounds (2) to (11)

Using the compounds shown in Table 1 with the molar ratio indicated in Table 1, polymeric compounds (2) to (11) were obtained in the same manner as in Synthesis Example 1.

With respect to the obtained polymeric compounds (1) to (11), the compositional ratio of the polymers (the molar ratio of the respective structural units in the polymeric compound) as determined by ¹³C-NMR, the weight average molecular weight (Mw) and the polydispersity (Mw/Mn) determined by the polystyrene equivalent value as measured by GPC are also shown in Table 1.

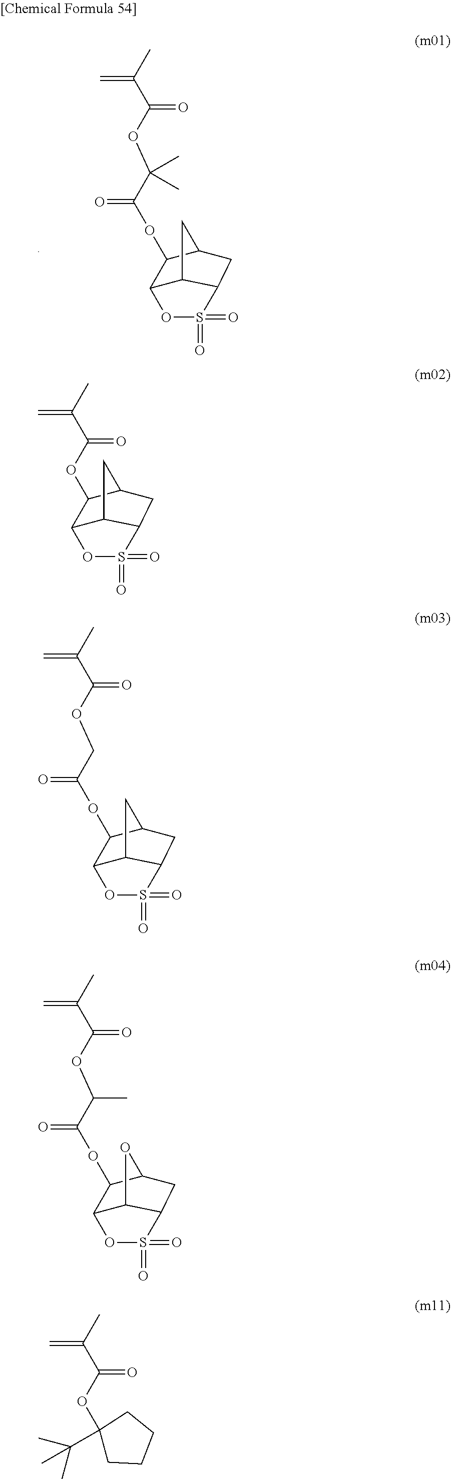

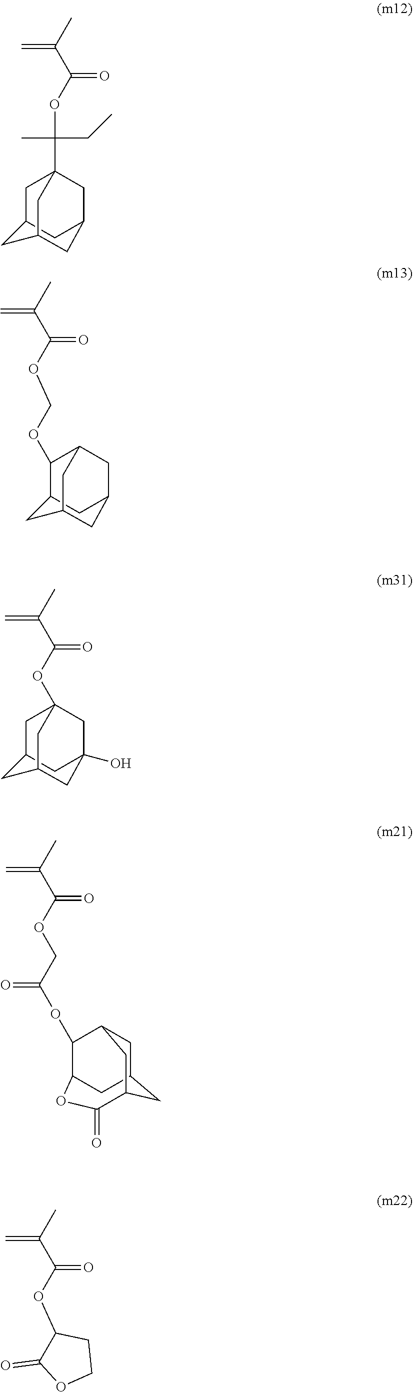

TABLE 1

| | Monomer | | | | | | | | | | | |
|---|---|---|---|---|---|---|---|---|---|---|---|---|
| Polymer | (m01) | (m02) | (m03) | (m04) | (m11) | (m12) | (m13) | (m21) | (m22) | (m31) | Mw | Mw/Mn |
| (1) | 42 | | | | 58 | | | | | | 10600 | 1.47 |
| (2) | 18 | | | | 51 | | | 21 | | 10 | 10100 | 1.47 |
| (3) | 41 | | | | | 59 | | | | | 9600 | 1.68 |
| (4) | 42 | | | | 39 | 19 | | | | | 10800 | 1.52 |
| (5) | 42 | | | | 28 | | 20 | | 10 | | 7200 | 1.58 |
| (6) | | 42 | | | 58 | | | | | | 9600 | 1.60 |
| (7) | | | 40 | | 60 | | | | | | 10600 | 1.66 |
| (8) | | | 19 | | 49 | | | 22 | | 10 | 10100 | 1.65 |
| (9) | | | | 21 | 48 | | | 21 | | 10 | 11100 | 1.61 |
| (10) | | | 41 | | 41 | 38 | | | | | 10300 | 1.68 |
| (11) | | | 41 | | 27 | | 21 | | 11 | | 6800 | 1.69 |

<Production of Resist Composition>

The components shown in Table 2 were mixed together and dissolved to obtain each resist composition.

TABLE 2

| | | | Component | Component | Component | | |
|---|---|---|---|---|---|---|---|
| | Component (A) | | (B) | (D) | (F) | Component (S) | |
| Example 1 | (A)-1 | | (B)-1 | (D)-1 | (F)-1 | (S)-1 | (S)-2 |
| | [100] | | [6.0] | [3.0] | [1.5] | [2750] | [10] |
| Example 2 | (A)-2 | | (B)-1 | (D)-1 | (F)-1 | (S)-1 | (S)-2 |
| | [100] | | [6.0] | [3.0] | [1.5] | [2750] | [10] |
| Example 3 | (A)-3 | | (B)-1 | (D)-1 | (F)-1 | (S)-1 | (S)-2 |
| | [100] | | [6.0] | [3.0] | [1.5] | [2750] | [10] |
| Example 4 | (A)-1 | (A)-3 | (B)-1 | (D)-1 | (F)-1 | (S)-1 | (S)-2 |
| | [33] | [67] | [6.0] | [3.0] | [1.5] | [2750] | [10] |
| Example 5 | (A)-4 | | (B)-1 | (D)-1 | (F)-1 | (S)-1 | (S)-2 |
| | [100] | | [6.0] | [3.0] | [1.5] | [2750] | [10] |
| Example 6 | (A)-5 | | (B)-1 | (D)-1 | (F)-1 | (S)-1 | (S)-2 |
| | [100] | | [6.0] | [3.0] | [1.5] | [2750] | [10] |
| Comparative | (A)-6 | | (B)-1 | (D)-1 | (F)-1 | (S)-1 | (S)-2 |
| Example 1 | [100] | | [6.0] | [3.0] | [1.5] | [2750] | [10] |
| Comparative | (A)-7 | | (B)-1 | (D)-1 | (F)-1 | (S)-1 | (S)-2 |
| Example 2 | [100] | | [6.0] | [3.0] | [1.5] | [2750] | [10] |
| Comparative | (A)-8 | | (B)-1 | (D)-1 | (F)-1 | (S)-1 | (S)-2 |
| Example 3 | [100] | | [6.0] | [3.0] | [1.5] | [2750] | [10] |
| Comparative | (A)-9 | | (B)-1 | (D)-1 | (F)-1 | (S)-1 | (S)-2 |
| Example 4 | [100] | | [6.0] | [3.0] | [1.5] | [2750] | [10] |
| Comparative | (A)-10 | | (B)-1 | (D)-1 | (F)-1 | (S)-1 | (S)-2 |
| Example 5 | [100] | | [6.0] | [3.0] | [1.5] | [2750] | [10] |
| Comparative | (A)-11 | | (B)-1 | (D)-1 | (F)-1 | (S)-1 | (S)-2 |
| Example 6 | [100] | | [6.0] | [3.0] | [1.5] | [2750] | [10] |

In Table 2, the reference characters indicate the following. The values in brackets [ ] indicate the amount (in terms of parts by weight) of the component added.

(A)-1 to (A)-11: the aforementioned polymers (1) to (11)

(B)-1: an acid generator represented by chemical formula (B)-1 shown below

(D)-1: acid diffusion control agent represented by chemical formula (D)-1 below

(F)-1: fluorine-containing polymeric compound represented by chemical formula (F)-1 below. The weight average molecular weight (Mw) and the dispersity (Mw/Mn) in terms of the polystyrene equivalent value measured by gel permeation chromatography (GPC) were 15,000 and 1.69, respectively. The composition of the copolymer (ratio (molar ratio) of the respective structural units within the structural formula) as determined by ¹³C-NMR was l/m=50/50.

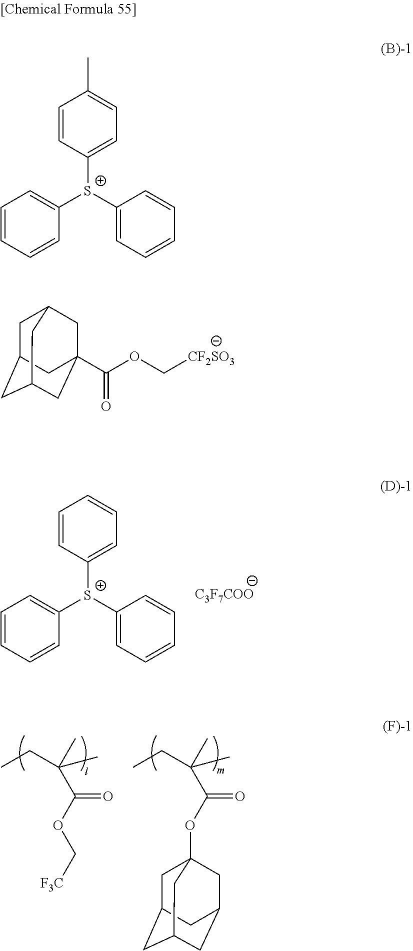

<Evaluation of Resist Composition>

Using the obtained resist composition, a resist pattern was formed, and the dissolution rate (Rₘₐₓ), the sensitivity (Eop) and the in-plane uniformity (CDU) of pattern size were evaluated as follows.

[Formation of Resist Pattern]

Each resist composition solution obtained in Examples 1 to 6 and Comparative Examples 1 to 6 was applied to a 12-inch silicon wafer, and the solution was then prebaked (PAB) and dried on a hot plate at 90° C. for 60 seconds, so as to form a resist film with a film thickness of 100 nm.

Subsequently, the resist film was selectively irradiated with an ArF excimer laser (193 nm) through an LS mask, using an immersion lithography ArF exposure apparatus NSR—S610C (manufactured by Nikon Corporation; NA (NA1.30, Dipole (in/out=0.78/0.97); immersion medium:water).

Then, a post exposure bake (PEB) treatment was conducted at 85° C. for 60 seconds.

Then, a solvent development was conducted at 23° C. for 13 seconds using butyl acetate. Thereafter, rinsing was conducted for 1 minute using methyl isobutyl carbinol (MIBC).

As a result, in the case where a resist composition of Examples 1 to 6 and Comparative Examples 2 to 6 was used, a contact hole pattern (hereafter, referred to as “CH pattern”) having a hole diameter of 45 nm and a pitch of 88 nm (mask size: 53 nm) was formed.

On the other hand, in the case where the resist composition of Comparative Example 1 was used, a pattern could not be resolved.

Further, regarding the pattern formed using the resist composition of Comparative Example 6, a very large number of resist residues were observed.

[Evaluation of Optimum Exposure Dose (Eop)]

The optimum exposure dose Eop (mJ/cm²) with which the target CH pattern was formed in the above “formation of resist pattern” was determined. The results are indicated under “Eop (mJ/cm²)” in Table 3.

[Evaluation of in-Plane Uniformity (CDU) of Pattern Size]

With respect to each hole pattern obtained above, 100 holes in the CH pattern were observed from the upper side thereof using a lengthwise measuring scanning electron microscope (SEM) (product name: S-9380, manufactured by Hitachi High-Technologies Corporation; acceleration voltage: 300V), and the hole diameter (nm) of each hole was measured.

From the results, the value of 3 times the standard deviation (σ) (3σ) was determined. The results are indicated under “CDU (nm)” in Table 3.

The smaller the thus determined 3σ value is, the higher the level of the dimension uniformity (CD uniformity) of the holes formed in the resist film.

[Evaluation of Dissolution Rate (Rₘₐₓ)]

Each resist composition of Examples 1 to 6 and Comparative Examples 1 to 6 was applied to a 12-inch silicon wafer using a spinner, and was then prebaked (PAB) on a hot plate at a bake temperature of 90° C. for 60 seconds, so as to form a resist film having a film thickness of 100 nm.

The obtained resist film was immersed in butyl acetate at 23° C. for 10 seconds, and the maximum value of the dissolution rate (amount of film thickness loss/immersion time) (Rₘₐₓ, unit:nm/s) was determined using RDA-808RB (product name; manufactured by Lithotec Japan). The results are shown in Table 3.

TABLE 3

| | Eop | CDU | Rmax |
|---|---|---|---|
| | (mJ/cm²) | (nm) | (nm/sec) |
| Example 1 | 28 | 2.87 | 30 |
| Example 2 | 30 | 2.83 | 33 |
| Example 3 | 16 | 3.21 | 69 |
| Example 4 | 28 | 2.92 | 31 |
| Example 5 | 22 | 2.82 | 42 |
| Example 6 | 18 | 3.56 | 22 |
| Comparative | — | — | <5 |
| Example 1 | | | |
| Comparative | 38 | 3.08 | 16 |
| Example 2 | | | |
| Comparative | 35 | 3.1 | 19 |
| Example 3 | | | |
| Comparative | 40 | 3.33 | 70 |
| Example 4 | | | |
| Comparative | 33 | 3.05 | 27 |
| Example 5 | | | |
| Comparative | 24 | 4.83 | 9 |
| Example 6 | | | |

From the results shown in Table 3, it was confirmed that the resist compositions of Examples 1 to 6 applying the present invention exhibited good dissolution rate (Rₘₐₓ) and also good CDU.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

1. A resist composition which generates acid upon exposure and exhibits changed solubility in a developing solution under action of acid, the resist composition comprising:
a resin component (A1) which exhibits changed solubility in a developing solution under action of acid,
the resin component (A1) including a structural unit (a0) represented by general formula (a0-1) shown below: 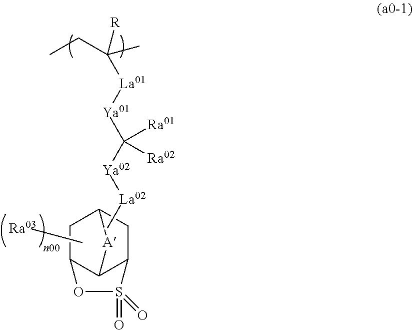
wherein R represents a hydrogen atom, an alkyl group of 1 to 5 carbon atoms or a halogenated alkyl group of 1 to 5 carbon atoms; La⁰¹ and La⁰² each independently represents a single bond or a divalent linking group; Ya⁰¹ and Ya⁰² each independently represents a single bond or an alkylene group of 1 to 3 carbon atoms, provided that the total number of carbon atoms of Ya⁰¹ and Ya⁰² is 3 or less; Ra⁰¹ and Ra⁰² each independently represents a hydrogen atom or an alkyl group of 1 to 4 carbon atoms; Ra⁰¹ and Ra⁰² may be mutually bonded to form a ring, provided that the total number of carbon atoms of Ra⁰¹ and Ra⁰² is 2 to 6; Ra⁰³ represents a hydrogen atom, an alkyl group, an alkoxy group, a halogen atom, a halogenated alkyl group, a hydroxy group, —COOR″, —OC(═O)R″, a hydroxyalkyl group or a cyano group; R″ represents a hydrogen atom, an alkyl group, a lactone-containing cyclic group, a carbonate-containing cyclic group or a —SO₂-containing cyclic group; n₀₀ represents an integer of 0 to 2; when n₀₀ is 2, the plurality of Ra⁰¹ may be the same or different from each other; and A′ represents an alkylene group of 1 to 5 carbon atoms.

2. A method of forming a resist pattern, comprising:
using a resist composition of claim 1 to form a resist film,
exposing the resist film, and
developing the exposed resist film using a developing solution containing an organic solvent to form a resist pattern.

3. A polymeric compound comprising a structural unit (a0) represented by general formula (a0-1) shown below: 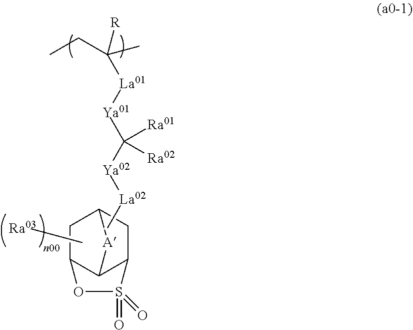
wherein R represents a hydrogen atom, an alkyl group of 1 to 5 carbon atoms or a halogenated alkyl group of 1 to 5 carbon atoms; La⁰¹ and La⁰² each independently represents a single bond or a divalent linking group; Ya⁰¹ and Ya⁰² each independently represents a single bond or an alkylene group of 1 to 3 carbon atoms, provided that the total number of carbon atoms of Ya⁰¹ and Ya⁰² is 3 or less; Ra⁰¹ and Ra⁰² each independently represents a hydrogen atom or an alkyl group of 1 to 4 carbon atoms; Ra⁰¹ and Ra⁰² may be mutually bonded to form a ring, provided that the total number of carbon atoms of Ra⁰¹ and Ra⁰² is 2 to 6; Ra⁰³ represents a hydrogen atom, an alkyl group, an alkoxy group, a halogen atom, a halogenated alkyl group, a hydroxy group, —COOR″, —OC(═O)R″, a hydroxyalkyl group or a cyano group; R″ represents a hydrogen atom, an alkyl group, a lactone-containing cyclic group, a carbonate-containing cyclic group or a —SO₂-containing cyclic group; n₀₀ represents an integer of 0 to 2; when n₀₀ is 2, the plurality of Ra⁰¹ may be the same or different from each other; and A′ represents an alkylene group of 1 to 5 carbon atoms.

4. A compound represented by general formula (m-a0) shown below: 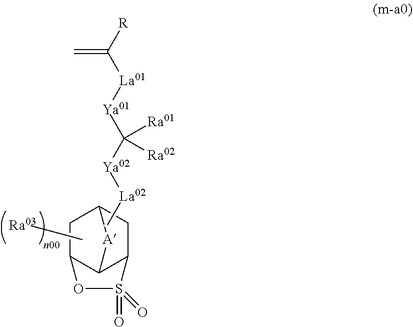
wherein R represents a hydrogen atom, an alkyl group of 1 to 5 carbon atoms or a halogenated alkyl group of 1 to 5 carbon atoms; La⁰¹ and La⁰² each independently represents a single bond or a divalent linking group; Ya⁰¹ and Ya⁰² each independently represents a single bond or an alkylene group of 1 to 3 carbon atoms, provided that the total number of carbon atoms of Ya⁰¹ and Ya⁰² is 3 or less; Ra⁰¹ and Ra⁰² each independently represents a hydrogen atom or an alkyl group of 1 to 4 carbon atoms; Ra⁰¹ and Ra⁰² may be mutually bonded to form a ring, provided that the total number of carbon atoms of Ra⁰¹ and Ra⁰² is 2 to 6; Ra⁰³ represents a hydrogen atom, an alkyl group, an alkoxy group, a halogen atom, a halogenated alkyl group, a hydroxy group, —COOR″, —OC(═O)R″, a hydroxyalkyl group or a cyano group; R″ represents a hydrogen atom, an alkyl group, a lactone-containing cyclic group, a carbonate-containing cyclic group or a —SO₂-containing cyclic group; n₀₀ represents an integer of 0 to 2; when n₀₀ is 2, the plurality of Ra⁰³ may be the same or different from each other; and A′ represents an alkylene group of 1 to 5 carbon atoms.

5. The resist composition according to claim 1, wherein the structural unit (a0) is represented by general formula (a0-11) shown below: 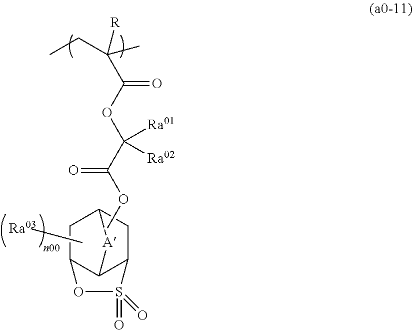
wherein R represents a hydrogen atom, an alkyl group of 1 to 5 carbon atoms or a halogenated alkyl group of 1 to 5 carbon atoms; Ra⁰¹ and Ra⁰² each independently represents a hydrogen atom or an alkyl group of 1 to 4 carbon atoms; Ra⁰¹ and Ra⁰² may be mutually bonded to form a ring, provided that the total number of carbon atoms of Ra⁰¹ and Ra⁰² is 2 to 6; Ra⁰³ represents a hydrogen atom, an alkyl group, an alkoxy group, a halogen atom, a halogenated alkyl group, a hydroxy group, —COOR″, —OC(═O) R″, a hydroxyalkyl group or a cyano group; R″ represents a hydrogen atom, an alkyl group, a lactone-containing cyclic group, a carbonate-containing cyclic group or a —SO₂-containing cyclic group; n₀₀ represents an integer of 0 to 2; when n₀₀ is 2, the plurality of Ra⁰³ may be the same or different from each other; and A′ represents an alkylene group of 1 to 5 carbon atoms.

6. The polymeric compound according to claim 3, wherein the structural unit (a0) is represented by general formula (a0-11) shown below: 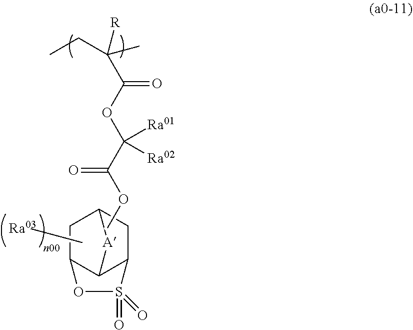
wherein R represents a hydrogen atom, an alkyl group of 1 to 5 carbon atoms or a halogenated alkyl group of 1 to 5 carbon atoms; Ra⁰¹ and Ra⁰² each independently represents a hydrogen atom or an alkyl group of 1 to 4 carbon atoms; Ra⁰¹ and Ra⁰² may be mutually bonded to form a ring, provided that the total number of carbon atoms of Ra⁰¹ and Ra⁰² is 2 to 6; Ra⁰³ represents a hydrogen atom, an alkyl group, an alkoxy group, a halogen atom, a halogenated alkyl group, a hydroxy group, —COOR″, —OC(═O) R″, a hydroxyalkyl group or a cyano group; R″ represents a hydrogen atom, an alkyl group, a lactone-containing cyclic group, a carbonate-containing cyclic group or a —SO₂-containing cyclic group; n₀₀ represents an integer of 0 to 2; when n₀₀ is 2, the plurality of Ra⁰³ may be the same or different from each other; and A′ represents an alkylene group of 1 to 5 carbon atoms.

7. The compound according to claim 4, which is represented by general formula (m-a0-1) shown below: 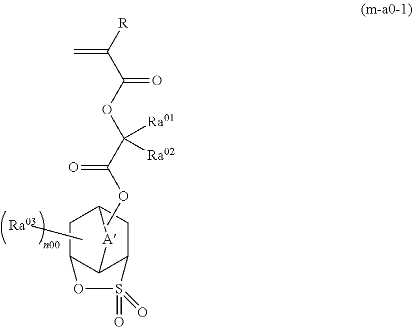
wherein R represents a hydrogen atom, an alkyl group of 1 to 5 carbon atoms or a halogenated alkyl group of 1 to 5 carbon atoms; Ra⁰¹ and Ra⁰² each independently represents a hydrogen atom or an alkyl group of 1 to 4 carbon atoms; Ra⁰¹ and Ra⁰² may be mutually bonded to form a ring, provided that the total number of carbon atoms of Ra⁰¹ and Ra⁰² is 2 to 6; Ra⁰³ represents a hydrogen atom, an alkyl group, an alkoxy group, a halogen atom, a halogenated alkyl group, a hydroxy group, —COOR″, —OC(═O) R″, a hydroxyalkyl group or a cyano group; R″ represents a hydrogen atom, an alkyl group, a lactone-containing cyclic group, a carbonate-containing cyclic group or a —SO₂-containing cyclic group; n₀₀ represents an integer of 0 to 2; when n₀₀ is 2, the plurality of Ra⁰³ may be the same or different from each other; and A′ represents an alkylene group of 1 to 5 carbon atoms
